(19) United States
(12) Patent Application Publication
(10) Pub. No.: US 2022/0184003 A1
(43) Pub. Date: Jun. 16, 2022

(54) Formulations of Cannabidiol Derivatives and Their Use as Modulators of Cannabinoid Receptor Type 2 (CB2)

(71) Applicant: EMERALD HEALTH PHARMACEUTICALS INC, San Diego, CA (US)

(72) Inventors: Alain ROLLAND, San Diego, CA (US); Eduardo Munoz BLANCO, Cordoba (ES)

(73) Assignee: EMERALD HEALTH PHARMACEUTICALS INC, San Diego, CA (US)

(21) Appl. No.: 17/428,929

(22) PCT Filed: Feb. 6, 2020
(86) PCT No.: PCT/US2020/017035

§ 371 Date: Aug. 5, 2021

Related U.S. Application Data
(60) Provisional application No. 62/870,546, filed on Jul. 3, 2019.
(60) Provisional application No. 62/801,756, filed on Feb. 6, 2019.

(51) Int. Cl. A61K 31/137; A61K 31/136; A61K 47/44; A61P 25/28; A61K 9/10

(52) U.S. Cl. CPC A61K 31/137; A61K 31/136; A61K 9/10; A61P 25/28; A61K 47/44

(57) ABSTRACT

Compositions, comprising the cannabidiol derivatives of Formula (I) in pharmaceutical formulations displaying increased bioavailability and solubility are described. Cannabidiol derivatives of Formula (I) and compositions comprising the same for use in the treatment of various conditions, and diseases, including diseases associated with demyelination.

### CROSS REFERENCE TO RELATED APPLICATIONS

This application claims priority to U.S. Provisional Application No. 62/801,756 filed Feb. 6, 2019 and U.S. Provisional Application No. 62/870,546, filed Jul. 3, 2019 which arc hereby incorporated by reference herein in their entirety.

### FIELD OF THE INVENTION

The present invention relates to compositions, comprising the cannabidiol derivatives of Formula (I) solubilized in pharmaceutical vehicle as liquid formulations, or a tablet, powder, suspension, nanosuspension, emulsion, which display increased bioavailability and solubility. The present invention also relates to the use of these cannabidiol quinone derivatives of Formula (I) for use in the treatment of diseases benefiting from the modulation of cannabinoid receptor type 2 (CB₂) activity. Such compounds have a novel mechanism of action (MOA) by targeting complementary signaling pathways that alleviate neuroinflammation and favor neuroprotection, prevent axonal damage, preserve and potentially promote the myelin structure, and support vasculogenesis, which is useful in the treatment of several autoimmune and inflammation-related disorders, including multiple sclerosis (MS) and systemic sclerosis (SSc).

### BACKGROUND OF THE INVENTION

Multiple sclerosis (MS) is a chronic autoimmune demyelinating disease of the central nervous system (CNS) that represents one of the most commonly acquired neurological diseases in young adults. Disease progression is thought to be composed of two underlying processes: myelin destruction (demyelination) with failure to remyelinate, and progressive axonal damage with little capacity for recovery. A variety of neurological symptoms associated with MS result from a weakening ability of the cells to conduct nerve signals. MS can cause disability progressively over time, including difficulty with mobility and upper limb function, bladder, bowel, and sexual dysfunction, speech and swallowing difficulties, and problems with vision and cognition. Currently, there is no curative treatment for MS, and standard of care mainly works on reducing symptoms. Since exacerbated innate and adaptive immune responses contribute to the pathophysiology of the disease, therapies that are directed towards modulation of the immune response and aimed at stimulation of axonal remyelination are needed. Systemic sclerosis (SSc), or scleroderma, is a group of rare diseases associated with early and transient inflammation and vascular injury, followed by fibrosis affecting the skin and multiple internal organs. Systemic sclerosis is classified into two forms: localized scleroderma (LoS) and SSc. While LoS is confined to the skin and/or underlying tissues and is often benign, SSc is a serious condition characterized by microvascular injury and SSc associated excessive fibrosis, which usually includes internal organ involvement. SSc may affect vital organs (heart kidneys, and lungs), other internal organs (stomach and bowels) as well as blood vessels, muscles and joints. As a result. SSc can lead to chronic debilitation and diminished life expectancy. Currently, there is no cure for SSc. Current therapies arc clinically ineffective, and available treatment options are organ and symptom specific.

Peroxisome proliferator-activated receptor gamma (PPARγ) and cannabinoid receptor type 2 (CB₂) are preclinically validated therapeutic targets, supported by scientific literature, for the development of novel drugs for the treatment, of MS (Docagne F. et al. 2008, Expert Opin. Ther. Targets., 12:185-195; Drew P. D. et al. 2008, PPAR Res. 2008:627463; Szalardy L et al. 2013, Neurosci Lett. 554:131-134). In addition, an activator of the hypoxia-inducible factor (HIF) pathway may have a beneficial effect in MS patients, as the HIF pathway modulates the immune response that favors neuroprotection and axonal regeneration and is responsible for postnatal myelination (Navarrete C et al. 2018. J Neuroinflammation, 15:64). There are classes of marketed drugs that activate one or the other of these pathways including Glitazones that activate PPARγ and cannabinoids that activate CB₂.

CB₂ receptors were first cloned from differentiated human HL-60 myeloid cells, and are most highly expressed in spleen, and cells of the immune system such as B cells, T cells, natural killer cells, macrophages, monocytes, and neutrophils. Lower levels of CB₂ receptors are also found in the epidermis (including keratinoeytes, hair follicles, sebocytes, and sweat glands), osteoblasts, osteoclasts, and osteocytes, as well as stomach, lung, heart and testis. CB₂ receptor expression has been reported in dorsal root ganglion (DRG), and evidence for CB₂ receptor expression in other peripheral neurons such as C- and Adelta-fibers has been reported. Recently CB₂ receptor expression within the CHS has been described, at both the spinal and supraspinal levels. Specifically, CB₂ receptors are found in lumbar (L3-L4) spinal cord, and in cerebellar granule neurons, cerebrovascular epithelium, microglia and neurons of the brainstem (striatum, thalamic nuclei, hippocampus, amygdala, substantia nigra, periaqueductal gray, spinal trigeminal nucleus etc.), cortex and cerebellum.

CB₂ receptors have been implicated in a number of physiological processes including inflammation and perception of pain, immune system regulation, neurogenesis, and bone physiology. Upregulation of CB₂ receptors is associated with certain pathophysiological states. Increased CB₂ receptor expression has been detected in dorsal horn of the spinal cord as well as primary afferent, C-fiber neurons in chronic constriction injury (CCl), spinal nerve ligation (SNL), complete sciatic nerve section, and saphenous nerve partial ligation models of neuropathic pain. CB₂ receptors are upreguiated in microglia and astrocytes from neoritic plaques found in Alzheimer's disease brains (Benito et al. 2003, J. Neurosci. 23:11136-11141), or by interferon gamma (Carlisle et al. 2002, Int. Immunopharmacol., 2:69-82) or lipopolysaccharide (Cabral et al. 2005, J. Leukoc. Biol., 78: 192-197), and in T-lymphoeytes from simian immunodeficiency vinis-infected macaques (Benito et al. 2005, J. Neurosci., 25:2530-2536). CB₂ receptors are found in T-lymphocytes, astrocytes and perivascular and reactive microglia in multiple sclerosis plaques (Benito et al. 2007, J. Neurosci., 27:2396-2402).

Myelin sheaths, which cover many nerve fibers, are composed of lipoprotein layers formed in early life. Myelin formed by the oligodendroglia in the central nervous system (CNS) differs chemically and immunoiogically from that formed by the Schwann cells peripherally, but both types have the same function: to promote transmission of a neural impulse along an axon. Many congenital metabolic disorders (e.g., phenylketonuria and other aminoacidurias; Tay-Sachs, Niemann-Pick, and Gaucher's diseases; Hurler's syndrome; Krabbe's disease and other leukody strophies) affect the developing myelin sheath, mainly in the CNS. Unless the biochemical defect can be corrected or compensated for, permanent, often widespread, neurologic deficits result.

Demyelination in later life is a feature of many neurologic disorders; it can result from damage to nerves or myelin due to local injury, ischemia, toxic agents, or metabolic disorders. Extensive myelin loss is usually followed by axonal degeneration and often by cell body degeneration, both of which may be irreversible. However, remyelination occurs in many instances, and repair, regeneration, and complete recovery of neural function can be rapid. Recovery often occurs after the segmental demyelination that characterizes many peripheral neuropathies; this process may account for the exacerbations and remissions of MS. Central demyelination (i.e., of the spinal cord, brain, or optic nerves) is the predominant finding in primary demyelmating diseases, whose etiology is unknown. The most well-known is MS. Other diseases include, for example, acute disseminated encephalomyelitis (postinfectious encephalomyelitis), adrenoleukodystrophy, adrenomydoneuropathy, Leber's hereditary optic atrophy and related mitochondrial disorders and human T-cell lymphotropic virus (HTLV) infection-associated myelopathy.

Remyelination is generally accepted as a regular event in MS lesions; however, it is insufficient for myelin repair and axons remain demyelinated in MS patients. Possible explanations for this include failure of recruitment or survival of oligodendrocyte progenitor cells (OPCs), disturbance of differentiation/maturation of OPCs. and loss of capability of myelin forming. Therefore, effective interventions for MS should not only prevent disease progression, but also promote remyelination.

There is a need in the art tor a disease-modifying drug, and a formulation thereof, with increased bioavailability and solubility to effort a more efficient drug delivery. There is also a need in the art for a disease-modifying drug, and a formulation thereof, with a novel mechanism of action (MOA) that targets complementary signaling pathways tliat alleviate neuroinflammation and favor both neuroprotection and myelin regeneration for management and treatment of various autoimmune diseases, demyelinating diseases, inflammatory-related disorders, and diseases of the central nervous system (CNS), such as MS and SSc.

### SUMMARY OF THE INVENTION

The invention provides compositions comprising at least one cannabidiol derivative solubilized in a pharmaceutical vehicle. In one aspect, the compositions have increased bioavailability. In another aspect, the compositions have increased solubility.

In one aspect, the cannabidiol derivatives, disclosed in the invention, are compounds of Formula (I).

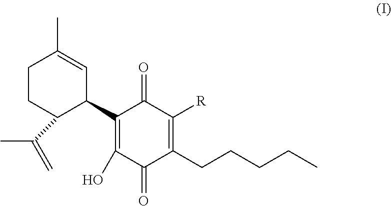

In one embodiment, R is the nitrogen atom of a group independently selected from a linear or branched alkylamine, an aryl amine, an arylalkylamine, a heteroarylamine. a heteroarylalkylamine, a linear or branched alkenylamine, a linear or branched alkynylamine, or NH₂.

In one embodiment, the composition is a dry powder formulation. In one embodiment, the composition is a tablet. In one embodiment, the composition is a suspension. In one embodiment, the composition is a nanosuspension. In one embodiment, the composition is an emulsion. In one embodiment, the composition is a solution.

In one embodiment, the pharmaceutical vehicle is selected from the group consisting of aqueous buffers, solvents, co-solvents, cyclodextrin complexes, lipid vehicles, and any combination thereof, and optionally further comprises at least one stabilizer, emulsifier, polymer, antioxidants, and any combination thereof.

In one aspect, the composition comprising at least one cannabidiol derivative of the invention, is solubilized in an oil. In some embodiments, the composition comprising at least one cannabidiol derivative of the invention, is solubilized in an oil mixture comprising at least two oils. In some embodiments, the composition comprising at least one cannabidiol derivative of the invention, is solubilized in a Maisine CC:maize oil mixture.

The invention also relates, in part, to a method of treating a condition or disease associated with demyelmation in a subject in need thereof. The invention further provides a method of treating a condition or disease responsive to a modulation of CB₂ activity in a subject. In one embodiment, the method comprises administering to the subject in need thereof a therapeutically effective amount of at least one cannabidiol derivative or a formulation thereof.

In some aspects, the invention relates to compositions comprising a non-reactive synthetic cannabidiol derivative has a novel mechanism of action (MOA) by targeting complementary signaling pathways that alleviate neuroinflammation and favor neuroprotection, prevent axonal damage, preserve myelin structure, and potentially promote remyelination. The compositions comprise a non-reactive synthetic cannabidiol derivative that modulates CB₂ receptor signaling. In some examples, the compositions comprise a non-reactive synthetic cannabidiol derivative that modulates both PPARγ and CB₂ receptor signaling. In some embodiments, the compositions comprise a non-reactive synthetic cannabidiol derivative that modulates PPARγ and CB₂ receptor signaling, and stabilizes HIF-1α, thus upregulating the expression of several associated factors that include Erythropoietin (EPO) and Vascular Endothelial Growth Factor A (VEGFA). As a result, such compositions can have a strong potential as disease-modifying agents in SSc.

The invention further relates, in part, to a method of remyelination in a subject in need thereof. In one aspect of the invention, the method comprises administering to the subject a therapeutically effective amount of at least one cannabidiol derivative or a formulation thereof. In one embodiment, the subject has a condition or disease associated with demyelination. In one embodiment, the subject has a condition or disease responsive to a modulation of CB₂ activity. In one embodiment, the subject has a condition or disease associated with demyelination and a condition or disease responsive to a modulation of CB₂ activity.

In one aspect, the condition or disease responsive to the modulation of the CB₂ receptor activity or the condition or disease associated with demyelination is selected from the group consisting of autoimmune disease, demyelinating disease, inflammatory-related disorder, and any combination thereof. In one embodiment, the condition or disease responsive to the modulation of the CB₂ receptor activity or the condition or disease associated with demyelination is selected from die group consisting of SSc, myelinodastic disorder, analgesia, acute and chronic pain, inflammatory pain, post-operative pain, neuropathic pain, muscle relaxation, immunosuppression, allergies, glaucoma, bronchodilation, osteoporosis and disorders of the skeletal system, cancer, neurodegenerative disorders including but not limited to Alzheimer's disease, Parkinson's disease (PD), and Huntington's disease. MS, muscle spasticity, tremor, fibromyalgia, lupus, rheumatoid arthritis, myasthenia gravis, other autoimmune disorders, irritable bowel syndrome, interstitial cystitis, migraine, pruriiis, eczema, seborrhea, psoriasis, shingles, cerebral ischemia, cerebral apoplexy, craniocerebral trauma, stroke, spinal cord injury, liver cirrhosis, atherosclerosis, coughing, asthma, nausea, emesis, gastric ulcers, neuromyelitis optica, central nervous system neuropathy, central pontine myelinolysis, myelopathy, leukoencephalopathy, leukodystrophy, peripheral neuropathy, Guillain-Barre syndrome, anti-MAG peripheral neuropathy, Charcot-Marie-Tooth disease, progressive inflammatory neuropathy, amyotrophic lateral sclerosis (ALS), and any combination thereof.

### BRIEF DESCRIPTION OF THE DRAWINGS

The following detailed description of various embodiments of the invention will be better understood when read in conjunction with the appended drawings. For the purpose of illustrating the invention, there are shown in the drawings illustrative embodiments. It should be understood, however, that the invention is not limited to the precise arrangements and instrumentalities of the embodiments shown in the drawings.

FIG. 1. comprising FIG. 1A and FIG. 1B, depicts synthetic schemes for the generation of cannabidiol derivatives. FIG. 1A represents the overall synthesis of amino functionalized cannabidiol derivative products produced front CBD starting material. FIG. 1B depicts the generation of VCE-004.8 (Compound of Formula (VIII)) via an amination of VCE-004.

FIG. 2 depicts a revised synthetic procedure for the generation of cannabidiol derivatives.

FIG. 3, comprising FIG. 3A and FIG. 3B. depicts optimization studies of various liquid formulation mixtures. FIG. 3A depicts different liquid formulation mixtures. FIG. 3B depicts a liquid formulation comprising 50:50 v/v of maize oil and Maisine CC mixture.

FIG. 4 depicts bioavailability of different liquid formulations.

FIG. 5, comprising FIGS. 5A and 5B, depicts manufacturing flow charts of EHP-101 liquid and placebo. FIG. 5A depicts a manufacturing flow chart of EHP-101 liquid. FIG. 5B depicts a manufacturing flow chart of placebo.

FIG. 6 depicts kinetic solubility screening of VCE-004.8.

FIG. 7 depicts an equation used to calculate log D (distribution coefficient) used as a measure of lipophilicity.

FIG. 8 depicts a stability of VCE-004.8 during phytosomization, at reflux in ethyl acetate at different times (45 min, 6 hr and 24 hr).

FIG. 9 depicts an overlay of the HPLC profiles of VCE-004.8 vs. the two phytosomes complex, obtained in the solubility trials at pH 7.4.

FIG. 10 depicts a dissolution profiles of formulations A, B and C of VCE-004.8 using Alitra.

FIG. 11 depicts solvent shift results in Simulated Gastric Fluid for various oral formulations.

FIG. 12 depicts solvent shift results in Simulated Intestinal Fluid for various oral formulations

FIG. 13 depicts a graphical representation of the Amorphous Solid Dispersion Screening and stability results.

FIG. 14 depicts a characterization of VCE-004.8 and EHP-101.

FIG. 15, comprising FIG. 15A through FIG. 15H. depicts the exemplary results that demonstrate that EHP-101 attenuates the clinical severity and neuropathology in BAH model. FIG. 15A depicts that EHP-101 significantly ameliorated the clinical signs and progression of EAE. Results are expressed as mean±SEM (n=6 animals per group). **p<0.01, ***p<0.001 EAE+BHP-101 vs EAE+VEH (one-way ANOVA followed Tukey's test). FIG. 15B depicts the results of clinical activity that, was quantified by measuring the area under curve. Results are expressed as ±SEM (n=6 to 11 animals per group). **p<0.01, ***p<0.001 EAE+EHP-101 vs EAE+Vehicle (one-way ANOVA followed Tukey's test). FIG. 15C depicts the cross-sectional images of thoracic spinal cord cross-sections of 50 μm thick, in which immunofluorescence with anti-Iba1 was performed. FIG. 15D depicts the cross-sectional images of thoracic spinal cord cross-sections of 50 μm thick, in which immunofluorescence with GFAP was performed. FIG. 15E depicts the cross-sectional images of thoracic spinal cord cross-sections of 50 μm thick, in which immunofluorescence with myelin staining MBP was performed. FIG. 15F depicts the results of quantification of Iba1 marker shown as mean±SEM, and significance was determined by one-way ANOVA followed Tukey's test ***p<0.001 EAE+Vehicle vs CFA; ##p<0.01, ###p<0.001 EAE+EHP-101 vs EAE+Vehicle. FIG. 15G depicts the results of quantification of GFAP marker shown as mean±SBM, and significance was determined by one-way ANOVA followed Tukey's test ***p<0.001 EAE+Vehicle vs CFA; ##p<0.01, ###p<0.001 EAE+EHP-101 vs EAE+Vehicle. FIG. 15H depicts the results of quantification of MBP marker shown as mean±SEM, and significance was determined by one way ANOVA followed Tukey's test ***p<0.001 EAE+Vehicle vs CFA; ##p<0.01, ###p<0.001 EAE+EHP-101 vs EAE+Vehicle.

FIG. 16, comprising FIG. 16A through FIG. 16H, depicts the exemplary results that demonstrate that demyelination with persistent activation of microglia and loss of Olig2 expression was prevented by EHP-101 treatment. The quantifications of each marker are shown as mean±SEM, and significance was determined by one way ANOVA followed Tukey's test *p<0.05, ***p<0.001 EAE+Vehicle vs CFA; #p−0.05, ##p<0.01, ###p<0.001 EAE+EHP-101 vs EAE+Vehicle. FIG. 16A depicts representative confocal microscopy images of cerebral corpus callosum immunolabeled for Iba1. FIG. 16B depicts representative confocal microscopy images of cerebral cortex showing that a reduced MBP reactivity was restored by EHP-101 treatment. FIG. 16C depicts representative confocal microscopy images that show that loss of Olig2 positive cells was prevented in EHP-101 treated mice. FIG. 16D depicts representative confocal microscopy images that show that EHP-101 treatment increased the expression of GSTpi in corpus callosum. FIG. 16E depicts the quantifications of Iba1 that is shown as mean±SEM, and significance was determined by one-way ANOVA followed by Tukey's test. *p<0.05, ***p<0.001 EAE+Vehicle vs CFA; #p<0.05, ##p<0.01, ###p<0.001 EAE+EHP-101 vs EAE+Vehicle. FIG. 16F depicts the quantifications of MBP that is shown as mean±SEM, and significance was determined by one-way ANOVA followed by Tukey's test. *p<0.05, ***p<0.001 EAE+Vehicle vs CFA; #p<0.05, ##p<0.01, ###p−0.001 EAE+EHP-101 vs EAE+Vehicle. FIG. 16G depicts the quantifications of Olig2 that is shown as mean±SEM. and significance was determined by one-way ANOVA followed by Tukey 's test *p<0.05, ***p<0.001 EAE+Vehicle vs CFA; #p<0.05, ##p<0.01, ###p<0.001 EAE+EHP-101 vs EAE+Vehicle. FIG. 16H depicts the quantifications of GSTpi that is shown as mean±SEM. and significance was determined by one-way ANOVA followed by Tukey's test. *p<0.05. ***p<0.001 EAE+Vehicle vs CFA; #p<0.05, ##p<0.01, ###p<0.001 EAE+EHP-101 vs EAE+Vehicle.

FIG. 17. comprising FIG. 17A through FIG. 17E, depicts the exemplary results of gene expression profiling of the effect of EHP-101 in EAE model. FIG. 17A depicts MA plots (MA plot is an application of a Bland-Altman plot for visual representation of genomic data) of the EAB or EAE+EHP-101 vs control comparisons. The X axis represents the averaged expression as the mean of normalized counts while the Y axis indicates the magnitude of the change as the log2 transformed fold change. The color indicates genes that surpassed the cutoff of adjusted P<0.05 and fold change<-2 (blue) or <2 (red). FIG. 17B depicts functional analysis results for genes that surpasses the previously mentioned cutoff in EAE vs Control and EAE+EHP-101 (20 mg/kg) vs EAE comparisons. The presence of a point indicates a significant over-representation (adjusted P<0.05) of Gene Ontology (Biological Process) term (Y axis) in a set of up or down regulates genes (X-axis). FIG. 17C depicts heatmap depicting the expression levels for selected genes included in the “cytokine-mediated signaling pathway”. FIG. 17D depicts heatmap showing the proteome profile of cytokines in CFA, EAE+vehicle and EAE+EHP-101 (20 mg/kg). FIG. 17E depicts the mRNA expression for inflammatory marker in spinal cord that was quantified by qPCR and normalized versus GAPDH. Data represent the mean±SEM, and significance was determined by one-way ANOVA followed Tukey's test *p<0.05, **p<0.01, ***p<0.001 EAE+Vehicle vs CFA; #p−0.05, ##p−0.01, ###p<0.001 EAE+EHP-101 vs EAE+Vehicle.

FIG. 18, comprising FIG. 18A through FIG. 18E, depicts the exemplary results that demonstrate that EHP-101 treatment normalized the expression of genes associated with oligodendrocyte function. FIG. 18A depicts Venn Diagram indicating the overlap bet ween the sets of down regulated genes at EAE vs Control comparison and up regulated genes at EAE+EHP-101 (20 mg/kg) vs EAE comparison. FIG. 18B depicts functional analysis results for the set of 193 overlapping genes. The scatter plot represents the significance of the enrichment for the top 15 over-represented Gene Ontology (Biological Process) terms as the −log 10 transformed adjusted P value. FIG. 18C depicts heatmap depicting the expression levels for genes annotated with the “myelination” GO term included in the set of 193 overlapping features. FIG. 18D depicts the mRNA expression for myelination related genes that was quantified by qPCR and normalized versus GAPDH. FIG. 18E depicts die results of immunohistochemistry labelling of spinal cord for Teneurin-4. The quantification of expression of Teneurin-4 in White/Grey matter (bottom panel). Data represents the mean±SEM. and significance was determined by one-way ANOVA followed Tukey's test **p<0.01, ***p<0.001 EAE+Vehicle vs CFA; #p−0.05, ##p<0.01, #190 #p<0.001 EAE+EHP-101 vs EAE+Vehicle.

FIG. 19, comprising FIG. 19A through FIG. 19E, depicts the effect of therapeutic EHP-101 treatment on remyelination in a Cuprizone (CPZ)-induced demyelination model. FIG. 19A depicts the experimental procedure used to evaluate the effect of therapeutic EHP-101 treatment on remyelination in a CPZ-induced demyelination model. FIG. 19B depicts the results of histological study of myelin by Cryomyelin staining in corpus callosum. FIG. 19C depicts the results that demonstrated a significant recover in myelin staining, which was shown by immunofluorescence studies of MBP in cortex. FIG. 19D depicts the mean intensity quantification results of histological study of myelin by Cryomyelin staining in corpus callosum (n=5 animals per group). FIG. 19B depicts the quantification of MBP immunoreactivity that, demonstrated a significant recover in myelin staining, which was shown by immunofluorescence studies of MBP in cortex. Dam represents the mean±SEM, and significance was determined by one-way ANOVA followed Tukey's test ***p<0.001 CPZ 6W or CPZ 6W+1 or CPZ 6W+2 vs Control; ###p<0.001 CPZ 6W+1+BHP-101 vs CPZ 6W+1; $$$p<0.001 CPZ 6W+2+EHP-101 vs CPZ 6W+2.

FIG. 20, comprising FIG. 20A through FIG. 20D, depicts the impact of therapeutic EHP-101 treatment on microglia and astrocytes activation in a CPZ-induced demyelination model. FIG. 20A depicts a decrease on cuprizone-induced microgliosis that was detected by immunofluorescence studies of Iba1 in corpus callosum. FIG. 20B depicts astrogliosis that was determined by immunofluorescence studies of GFAP in corpus callosum. FIG. 20C depicts a quantified decrease on cuprizone-induced microgliosis that was detected by immunofluorescence studies of Iba1 in corpus callosum. FIG. 20D depicts quantified intensity of astrogliosis that was determined by immunofluorescence studies of GFAP in corpus callosum. Data represents the mean±SEM, and significance was determined by one-way ANOVA followed Tukey's test ***p<0.001 CPZ 6W or CPZ 6W+1 or CPZ 6W+2 vs Control: **p<0.01 CPZ 6W+2 vs Control; ##p<0.01 CPZ 6W+1+EHP-101 vs CPZ 6W+1.

FIG. 21 depicts representative primers used in real-time PCR analysis.

FIG. 22, comprising FIG. 22A and FIG. 22B, depicts representative results demonstrating that EHP-101 reduces axonal degeneration and plasma levels of neurofilament light polypeptide (NEFL). FIG. 22A depicts representative images of immunostaining of SMI-32+ cells in the Corpus callosum of different groups of animals. FIG. 22B depicts NEFL plasma levels were detected by ELISA in the different groups of animals. Values were normalized versus control group and correspond to mean±SEM and significance was determined by one-way ANOVA followed by Tukey's test. *p<0.05 CPZ 6W or CPZ 6W+1 vs Control; #p<0.05 CPZ 6W+1+EHP-101 vs CPZ 6W+1.

FIG. 23 depicts the experimental procedure used to evaluate the effect of therapeutic oral EHP-101 treatment on remyelination in a CPZ-induced demyelination model.

FIG. 24, comprising FIG. 24A through FIG. 24D, depicts grey matter (hippocampus) remyelination results. FIG. 24A depicts PLP staining in the hippocampus. FIG. 24B depicts quantification results of PLP in the hippocampus. EHP-101-treated animals showed no change in the area of PLP staining in the hippocampus compared to vehicle control. FIG. 24C depicts quantification results of PLP in the hippocampus. Outliers were identified using Chauvenet's criterion. No outliers were excluded from statistical analysis. FIG. 24D depicts hippocampal statistics for PLP stain.

FIG. 25. comprising FIG. 25A through FIG. 25D, depicts grey matter (cortex) remyelination results. FIG. 25A depicts PLP staining in the cortex. FIG. 25B depicts quantification results of PLP in the cortex. EHP-101-treated animals at all dose strengths showed no change in die area of PLP staining in the cortical region compared to vehicle control. FIG. 25C depicts quantification of PLP in the cortex. Outliers were identified using Chauvenet's criterion. No outliers were excluded from statistical analysis. FIG. 25D depicts the statistics for PLP stain.

FIG. 26, comprising FIG. 26A through FIG. 26D, depicts white matter (corpus callosum) remyelination results. FIG. 26A depicts PPD staining in the corpus callosum. FIG. 26B depicts quantification results of PPD in the corpus callosum (without age matched (AM) sample); the myelinated axons in corpus callosum. Although EHP-101 treatments did not show a significant increase in myelinated axons compared to control, there was a significant difference between the two higher groups when compared to the lowest tested group of the test article. FIG. 26C depicts quantification of PPD in the corpus callosum. Outliers were identified using Chauvenet's criterion. Sample 44 was excluded from statistical analysis. FIG. 26D depicts number of myelinated axons in corpus callosum statistics (without AM sample).

FIG. 27, comprising FIG. 27A and FIG. 27B, depicts white matter (corpus callosum) remyelination results (with AM sample). FIG. 27A depicts quantification results of PPD in corpus callosum; the myelinated axons in corpus callosum (with AM sample). Although EHP-101 treatments did not show a significant increase in myelinated axons compared to control, there was a significant difference between the two higher groups when compared to the lowest tested group of the test article. FIG. 27B depicts number of myelinated axons in corpus callosum statistics (with AM sample).

FIG. 28, comprising FIG. 28A and FIG. 28B, depicts white matter (corpus callosum) remyelination results (without AM sample). FIG. 28A depicts the density of myelinated axons (PPD density) in corpus callosum (without AM sample). The higher doses tested of EHP-101 treatments showed a significant increase in the density of myelinated axons compared to control, there w as also a significant difference between the two higher groups when compared to the lowest tested group of the test article. FIG. 28B depicts the statistics for the density of myelinated axons in corpus callosum (without AM sample).

FIG. 29, comprising FIG. 29A and 29B, depicts white matter (corpus callosum) remyelination results (with AM sample). FIG. 29A depicts the density of myelinated axons (PPD density) in corpus callosum (with AM sample). The higher doses tested of EHP-101 treatments showed a significant increase in the density of myelinated axons compared to control, there was also a significant difference between the two higher groups when compared to the lowest tested group of the test article. FIG. 29B depicts the statistics for the density of myelinated axons in corpus callosum (with AM sample).

### DETAILED DESCRIPTION

It is to be understood that the Figures and descriptions of the present invention have been simplified to illustrate elements that are relevant for a clear understanding of the present invention, while eliminating, for the purpose of clarity, many other elements found in the method of treating a condition or disease responsive to a modulation of CB₂ activity or a condition or disease associated with demyelination using the compound of Formula (I) as well as methods of making and using such compounds, pharmaceutical compositions, and liquid formulations thereof. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

As used herein, each of the following terms has the meaning associated with it in this section. Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent depending on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

A disease or disorder is “alleviated” if the severity of a sign or symptom of the disease or disorder, the frequency with which such a sign or symptom is experienced by a patient, or both, is reduced.

A “disease” is a state of health of an animal wherein the animal cannot maintain homeostasis, and wherein if the disease is not ameliorated then the animal's health continues to deteriorate, in contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “inhibit,” as used herein, means to suppress or block an activity or function by at least about ten percent relative to a control value. Preferably, the activity is suppressed or blocked by 50% compared to a control value, more preferably by 75%, and even more preferably by 95%.

In the context of the present disclosure, a “modulator” is defined as a compound that is an agonist, a partial agonist, an inverse agonist or an antagonist of CB₂. A modulator may increase the activity of the CB₂ receptor, or may decrease the activity of the CB₂ receptor. In the context of the present disclosure, an “agonist” is defined as a compound that increases the basal activity of a receptor (i.e., signal transduction mediated by the receptor). An “antagonist” is defined as a compound, which blocks the action of an agonist on a receptor. A “partial agonist” is defined as an agonist that displays limited, or less than complete, activity such that it fails to activate a receptor in vitro, functioning as an antagonist in vivo. An “inverse agonist” is defined as a compound that decreases the basal activity of a receptor.

The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a subject and includes: (a) preventing a disease related to an undesired immune response from occurring in a subject which may be predisposed to the disease: (b) inhibiting the disease, i.e., arresting its development: or (c) relieving the disease, i.e., causing regression of the disease.

The term “derivative” refers to a small molecule that differs in structure from the reference molecule, but may retain or enhance the essential properties of the reference molecule and may have additional properties. A derivative may change its interaction with certain other molecules relative to the reference molecule. A derivative molecule may also include a salt, an adduct, tautomer, isomer, or other variant of the reference molecule.

The term “tautomers” are constitutional isomers of organic compounds that readily interconvert by a chemical process (tautomerization).

The term “isomers” or “stereoisomers” refers to compounds, which have identical chemical constitution, but differ with regard to the arrangement of the atoms or groups in space.

As used herein “polymorph” refers to crystalline forms having the same chemical composition but different spatial arrangements of the molecules, atoms, and/or ions forming the crystal.

As used herein, “alkyl” refers to a linear or branched chain fully saturated (no double or triple bonds) hydrocarbon (all carbon) group. An alkyl group of this invention may comprise from 1-20 carbon atoms, that is, “m”=1 and “n”=20, designated as a “C₁ to C₁₀ alkyl.” In one embodiment, “m”=1 and “n”=12 (C₁ to C₁₂ alkyl). In other embodiments, that “m”=1 and “n”=6 (C₁ to C₆ alkyl). Examples of alkyl groups include, without limitation, methyl, ethyl, n-propy), isopropyl, n-butyl, iso-butyl, sec-butyl, tert-butyl, amyl, tert-amyl, hexyl, heptyl, octyl, nonyl, decyl, undecyl, and dodecyl.

An alkyl group of this invention may be substituted or unsubstitutcd. When substituted, the substituent group(s) is(are) one or more group(s) independently selected from cycloalkyl, aryl, heteroaryl, heteroalicyclyl, hydroxy, alkoxy, aryloxy, mercapto, alkylthio, arylthio, cyano, halo, oxo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, —NRaRb, protected hydroxyl, protected amino, protected carboxy, and protected amido groups.

Examples of substituted alkyl groups include, without limitation, 2-oxo-prop-1-yl, 3-oxo-but-1-yl, cyanomethyl, nitromethyl, chloromethyl, hydroxymethyl, tetrahydropyranyloxymethyl, m-trityloxymethyl, propionyloxymethyl, aminomethyl, carboxymethyl, allyloxycarbonylmethyl, allyloxycarbonylaminomethyl, methoxymethyl, ethoxymethyl, t-butoxymethyl, acetoxymethyl, chloromethyl, bromomethyl, iodomethyl, trifluoromethyl, 6-hydroxyhexyl, 2,4-dichlorobmyl, 2-aminopropyl, 1-chloroethyl, 2-chloroethyl, 1-bromoethyl, 2-chloroethyl, 1-fluoroethyl, 2-fluoroethyl, 1-iodoethyl, 2-iodoethyl, 1-chloropropyl, 2-chloropropyl, 3-chloropropyl, 1-bromopropyl, 2-bromopropyl, 3-bromopropyl, 1-fluoropropyl, 2-fluoropropyl, 3-fluoropropyl, 1-iodopropyl, 2-iodopropyl, 3-iodopropyl, 2-aminoethyl, 1-aminoedtyl, N-benzoyl-2-aminoethyl, Nacetyl-2-aminoethyl, N-benzoyl-1-aminoethyl, and N-acetyl-1-aminoethyl.

As used herein, “alkenyl” refers to an alkyl group that contains in a linear or branched hydrocarbon chain one or more double bonds. Examples of alkenyl groups include, without limitation, vinyl (CH₂═CH—), allyl (CH₃CH═CR₂—), 1-propenyl, 2-propenyl, 1-butenyl, 2-butenyl; 1-pentenyl, 2-pentenyl, 3-pentenyl, 4-pentenyl, 3-methyl-1-butenyl, and the various isomers of hexenyl, heptenyl, octenyl, nonenyl, decenyl, undecenyl, and dodecenyl.

An alkenyl group of this invention may be unsubstituted or substituted. When substituted, the substituent(s) may be selected from the same groups disclosed above with regard to alkyl group substitution. Examples of substituted alkenyl groups include, without limitation, styrenyl, 3-chloro-propen-1-yl, 3-chloro-buten-1-yl, 3-methoxy-propen-2-yl, 3-phenyl-buten-2-yl, and 1-cyano-buten3-yl.

As used herein, “alkynyl” refers to an alkyl group that contains in a linear or branched hydrocarbon chain one or more triple bonds.

An alkynyl group of this invention may be unsubstituted or substituted. When substituted, the substituem(s) may be selected from the same groups disclosed above with regard to alkyl group substitution.

As used herein, “aryl” refers to a carbocyclic (all carbon) ring or two or more fused rings (rings that share two adjacent carbon atoms) that have a fully delocalized pi-electron system. Examples of aryl groups include, but are not limited to, benzene, and substituted benzene, such as toluene, aniline, xylene, and the like, naphthalene and substituted naphthalene, and azulene.

The term “pharmaceutically acceptable salt” refers to any pharmaceutically acceptable salt, which upon administration to the patient is capable of providing (directly or indirectly) a compound as described herein. Such salts preferably arc acid addition salts with physiologically acceptable organic or inorganic acids. Examples of the acid addition salts include mineral acid addition salts such as, for example, hydrochloride, hydrobromide, hydroiodide, sulphate, nitrate, phosphate, and organic acid addition salts such as, for example, acetate, trifluoroacetate, maleate, fumarate, citrate, oxalate, succinate, tartrate, malate, mandelate, methane sulphonale and p-toluenesulphonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic amino acids salts. However, it will be appreciated that non-pharmaceutically acceptable salts also fall within the scope of the invention since those may be useful in the preparation of pharmaceutically acceptable salts. Procedures for salt formation are conventional in the art.

The term “solvate” in accordance with this invention should be understood as meaning any form of the active compound in accordance with the invention in which said compound is bonded by a non-covalent bond to another molecule (normally a polar solvent), including especially hydrates and alcoholates.

The terms “effective amount” and “pharmaceutically effective amount” refer to a sufficient amount of an agent to provide the desired biological result. That result can be reduction and/or alleviation of a sign, symptom, or cause of a disease or disorder, or any other desired alteration of a biological system. An appropriate effective amount in any individual case may be determined by one of ordinary skill in the art using routine experimentation.

A “therapeutically effective amount” refers to that amount which provides a therapeutic effect for a given condition and administration regimen. In particular, “therapeutically effective amount” means an amount that is effective to prevent, alleviate or ameliorate symptoms of the disease or prolong the survival of the subject being treated, which may be a human or non-human animal. Determination of a therapeutically effective amount is within the skill of the person skilled in the art.

As used herein, the term “pharmaceutical composition” refers to a mixture of at least one compound of the invention with other chemical components and entities, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

“Pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredients) and is not toxic to the host to which it is administered.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as com starch and potato starch; cellulose, and its derivatives, such as sodium carboxy methyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid: pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

The term “nutritional composition” may be a food product intended for human consumption, for example, a beverage, a drink, a bar, a snack, an ice cream, a dairy product, for example a chilled or a shelf-stable dairy product, a fermented dairy product, a drink, for example a milk-based drink, an infant formula, a growing-up milk, a confectionery product, a chocolate, a cereal product such as a breakfast cereal, a sauce, a soup, an instant drink, a frozen product intended for consumption after heating in a microwave of an oven, a ready-to-eat product, a fast food or a nutritional formula.

The terms “patient” “subject,” “individual ” and the like are used interchangeably herein, and refer to any animal, or cells thereof whether in vitro or in situ, amenable to the methods described herein. In certain non-limiting embodiments, the patient, subject or individual is a human.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3.4, 5, 5.3, and 6. This applies regardless of the breadth of the range.

### Description

### Formulation/Pharmaceutical

The invention provides a composition comprising at least one cannabidiol derivative solubilized in a pharmaceutical vehicle. In one embodiment, the composition has increased bioavailability. In one embodiment, the composition has increased bioavailability when compared to the bioavailability of the same cannabidiol derivative in a non-formulated mixture. In one embodiment, the composition has increased solubility. In one embodiment, die composition has improved solubility when compared to the solubility of the same cannabidiol derivative in a non-formulated mixture.

In one embodiment, the composition is a dry powder formulation. In one embodiment, the composition is a tablet, wherein the tablets, comprising the cannabidiol derivatives, are prepared through two manufacturing steps: a granulation step and a tablet preparation step. In one embodiment, the granulation step is a preparation of the intermediate product (IP). In one embodiment, the granulation step comprises a granulating fluid containing excipients in ethanol that is added to primary powder particles and followed by solvent evaporation. In one embodiment, the particle size of the resulting material is reduced by milling. In one embodiment, the tablet preparation step is a preparation of the Drug Product (DP). In one embodiment, an intermediate product (IP), wherein the intermediate product (IP) is obtained from the granulation step, is blended with excipients. In one embodiment, the Drug Product (DP) is tablet compressed by direct compression on a tablet press.

In one embodiment, the composition is a suspension. In one embodiment, the composition is a nanosuspension. In one embodiment, the composition is an emulsion. In one embodiment, the composition is a solution. In one embodiment, the composition is a liquid formulation. In one embodiment, the composition is a cream. In one embodiment, the composition is a gel. In one embodiment, the composition is a lotion. In one embodiment, the composition is a paste. In one embodiment, the composition is an ointment. In one embodiment, the composition is an emollient. In one embodiment, the composition is a liposome. In one embodiment, the composition a nanosphere. In one embodiment, the composition is a skin tonic. In one embodiment, the composition is a mouth wash. In one embodiment, the composition is an oral rinse. In one embodiment, the composition is a mousse, in one embodiment, the composition is a spray. In one embodiment; the composition is a pack. In one embodiment, the composition is a capsule. In one embodiment, the composition is a tablet. In one embodiment, the composition is a powder. In one embodiment, the composition is a granule. In one embodiment, the composition is a patch. In one embodiment, the composition an occlusive skin agent.

In one embodiment, the composition comprises new drug candidates comprising chemically stable, nonpsychotropic aminoquinoid chemically derived from synthetic or natural cannabidiol (CBD) through oxidation and animation. In one embodiment, the cannabidiol derivative is a synthetic cannabidiol derivative. In one embodiment, the synthetic cannabidiol derivative comprises chemically stable, nonpsychotropic aminoquinoid chemically derived from synthetic cannabidiol (CBD) through oxidation and animation. In one embodiment, the synthetic cannabidiol derivative comprises chemically stable, nonpsychotropic aminoquinoid chemically derived from natural cannabidiol (CBD) through oxidation and amination. In one embodiment, the synthetic cannabidiol derivative is a non-reactive synthetic cannabidiol derivative. In one embodiment, the non-reactive synthetic cannabidiol derivative is a chemically stable synthetic cannabidiol derivative. In one embodiment, the non-reactive synthetic cannabidiol derivative is a synthetic cannabidiol derivative that does not have a detectable affinity for the CBI receptor.

In one embodiment, the composition comprising a non-reactive synthetic cannabidiol derivative has a novel mechanism of action (MOA) by targeting complementary signaling pathways that alleviate neuroinflammation and favor neuroprotection, prevent axonal damage, preserve myelin structure, and potentially promote remyelination. In one embodiment, the composition comprising a non-reactive synthetic cannabidiol derivative is a modulator of CB₂ receptor signaling. In one embodiment, the composition comprising a non-reactive synthetic cannabidiol derivative is a modulator of PPARγ and CB₂ receptor signaling. In one embodiment, the composition comprising a non-reactive synthetic cannabidiol derivative is a modulator of PPARγ and CB₂ receptor signaling, and stabilizes HIF-1α thus upregulating the expression of several associated factors that include Erythropoietin (EPO) and Vascular Endothelial Growth Factor A (VEGFA). In one embodiment, the composition comprising a non-reactive synthetic cannabidiol derivative reduces neuroinflammation presumably by acting on PPARγ/CB₂ receptors, in conjunction with enhanced neuroprotection and potential remyelination through the HIF pathway.

In one embodiment, the composition comprising a non-reactive synthetic cannabidiol derivative binds the CB₂. In one embodiment, the non-reactive synthetic cannabidiol derivative preferentially binds to CB₂ receptor as compared to cannabinoid receptor type I (CB₁). Therefore, in these embodiments, the non-reactive synthetic cannabidiol derivative is selective for CB₂. In one embodiment, the amine group of non-reactive synthetic cannabidiol derivative binds the CB₂. In one embodiment, the amine group of non-reactive synthetic cannabidiol derivative selectively binds the CB₂ receptor over the CB₁ receptor. In one embodiment, the CB₂ receptor activity is modulated in vitro, whereas in other embodiments, the CB₂ receptor activity is modulated in vivo.

In one embodiment, the cannabidiol derivative is a compound of Formula (I).

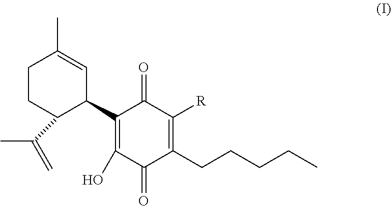

In one embodiment, R is the nitrogen atom of a group independently selected from a linear or branched alkylamine, an arylamine, an arylalkylamine, a heteroaryl amine, a heteroarylalkylamine, a linear or branched alkenylamine, a linear or branched alkynylamine, or NH₂.

In one embodiment, the cannabidiol derivative is selected from the group consisting of:

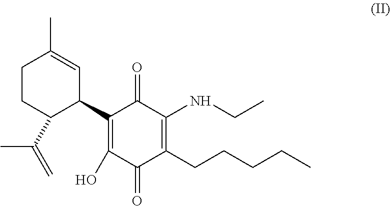

### (1′R,6′R)-3-(Ethylamine)-6-hydroxy-3′-methyl-4-pentyl-6′-(prop-1-en-2-yl)[1,1-bi(cyclohexane)]-2′,3,6-triene-2,5-dione

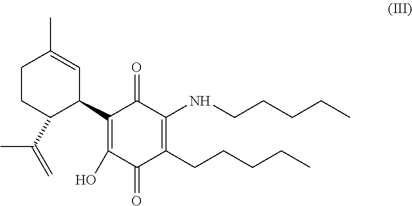

### (1′R,6′R)-3-(Pentylamine)-6-Hydroxy-3′-methyl-4-pentyl-6′-(prop-1-en-2-yl)-[1,1′bi(cyclohexane)]-2′,3,6-triene-2,5-dione

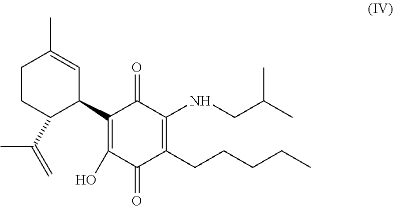

### (1′R,6′R)-3-(Isobutylamine)-6-Hydroxy-3′-methyl-4-pentyl-6′-(prop-1-en-2-yl)[1,1′-bi(cyclohexane)]-2′,3,6-triene-2,5-dione

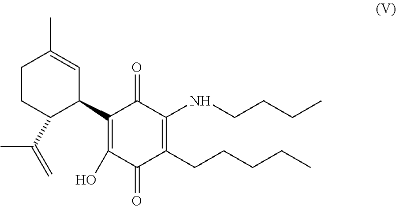

### (1′R,6′R)-3-(Butylamine)-6-hydroxy-3′-methyl-4-pentyl-6-(prop-1-en-2-yl)[1,1′-bi(cyclohexane)]-2′,3,6-triene-2,5-dione

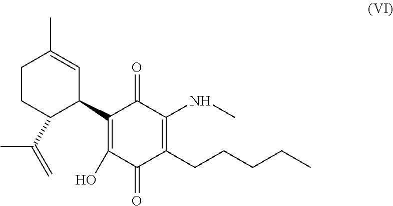

### (1′R,6′R)-3-(Methylamine)-6-Hydroxy-3′-methyl-4-pentyl-6′-(prop-1-en-2-yl)[1,1′-bi(cyclohexane)]-2′,3,6-triene-2,5-dione

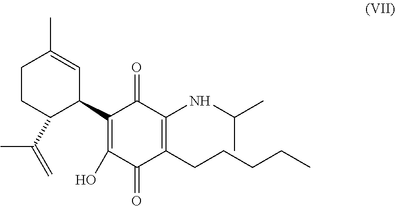

### (1′R,6′R)-3-(Isopropylamine)-6-Hydroxy-3′-methyl-4-pentyl-6′-(prop-1-en-2-yl)-[1,1′-bi(cyclohexane)]-2′,3,6-triene-2,5-dione

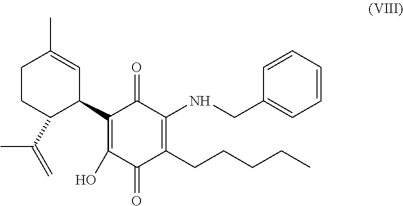

### (1′R,6′R)-3-(Benzylamine)-6-hydroxy-3′-methyl-4-pentyl-6′-(prop-1-en-2-yl)[1,1′-bi(cyclohexane)]-2′,3,6-triene-2,5-dione

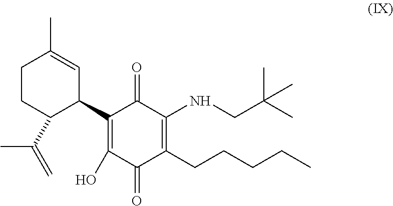

### (1′R,6′R)-3-(Neopentylamine)-6-hydroxy-3′-methyl-)-4-pentyl-6′-(prop-1-en-2yl)-[1,1′-bi(cyclohexane)]-2′,3,6-triene-2,5-dione, and

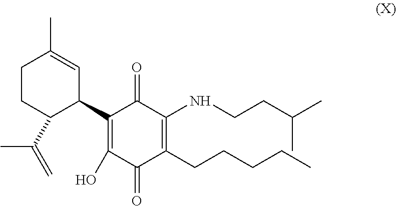

### (1′R,6′R)3-Isopentylamine)-6-Hydroxy-amine-3′-methyl-4-pentyl-6′-(prop-1-en-2-yl)-[1,1′-bi(cyclohexane)]-2′,3,6-triene-2,5-dione

In one embodiment, the pharmaceutical vehicle is selected from the group consisting of aqueous buffers, solvents, co-solvents, cyclodextrin complexes, lipid vehicles, and any combination thereof, and optionally further comprising at least one stabilizer, emulsifier, polymer, antioxidant, and any combination thereof.

In one embodiment, the aqueous buffer is selected from the group consisting of aqueous HCl, aqueous citrate-HCl buffer, aqueous NaOH. aqueous citrate-NaOH buffer, aqueous phosphate buffer, aqueous KCl, aqueous borate KCl-NaOH buffer, PBS buffer, and any combination thereof.

In one embodiment, the aqueous buffer has pH range of pH=0.5-10. In one embodiment, the aqueous buffer has pH range of pH=0.5. In one embodiment, the aqueous buffer has pH=1.0. In one embodiment, the aqueous buffer has pH=2.0. In one embodiment, the aqueous buffer has pH=3.0. In one embodiment, the aqueous buffer has pH=4.0. In one embodiment, the aqueous buffer has pH=5.0. In one embodiment, the aqueous buffer has pH=5.5. In one embodiment, the aqueous buffer has pH=6.0. In one embodiment, the aqueous buffer has pH=7.0. In one embodiment, the aqueous buffer has pH=7.4. In one embodiment, the aqueous buffer has pH=8.0. In one embodiment, the aqueous buffer has pH=9.0. In one embodiment, the aqueous buffer has pH=9.5. In one embodiment, the aqueous buffer has pH=10.0.

In one embodiment, the aqueous buffer has a concentration range of 0.05 N-1.0 N. In one embodiment, the aqueous buffer has a concentration of 0.05 N. In one embodiment, the aqueous buffer has a concentration of 0.1 N. In one embodiment, the aqueous buffer has a concentration of 0.15 N. In one embodiment, the aqueous buffer has a concentration of 0.2 N. In one embodiment, the aqueous buffer has a concentration of 0.3 N. In one embodiment, the aqueous buffer has a concentration of 0.4 N. In one embodiment, the aqueous buffer has a concentration of 0.5 N. In one embodiment, the aqueous buffer has a concentration of 0.6 N. In one embodiment, the aqueous buffer has a concentration of 0.7 N. In one embodiment, the aqueous buffer has a concentration of 0.8 N. In one embodiment, the aqueous buffer has a concentration of 0.9 N. In one embodiment, the aqueous buffer has a concentration of 1.0 N.

In one embodiment, the solvent is selected from the group consisting of acetone, ethyl acetate, acetonitrile, pentane, hexane, heptane, methanol, ethanol, isopropyl alcohol, dimethyl sulfoxide (DMSO), water, chloroform, dichloromethane, diethyl ether, PEG400, Transcutol (diethylene glycomonoethyl ether). MCT 70, Labrasol (PEG-8 caprylic/capric glycerides). Labrafil M1944CS (PEG 5 Oleate), propylene glycol, Transcutol P, PEG400, propylene glycol, glycerol, Captex 300, Tween 85, Cremophor EL, Maisine 35-1, Maisine CC, Capmul MCM, maize oil, and any combination thereof.

In one embodiment, the co-solvent is selected from the group consisting of acetone, ethyl acetate, acetonitrile, pentane, hexane, heptane, methanol, ethanol, isopropyl alcohol, dimethyl sulfoxide (DMSO), water, chloroform, dichloromethane, diethyl ether, PEG400, Transcutol (diethylene glycomonoethyl ether), MCT 70, Labrasol (PEG-8 caprylic/capric glycerides), Labrafil M1944CS (PEG 5 Oleate), propylene glycol, Transcutol P, PEG400, propylene glycol, glycerol, Captex 300, Tween 85, Cremophor EL, Maisine 35-1, Maisine CC, Capmul MCM, maize oil, and any combination thereof.

In one embodiment, the cyclodextrin complexes is selected from the group consisting of methyl-β-cyclodextrin, methyl-γ-cyclodextrin, HP-β-cyclodextrin, HP-γ-cyclodextrin, SBE-β-cyclodextrin, α-cyclodextrin, γ-cyclodextrin, 6-O-glucosyl-β-cyclodextrin, and any combination thereof.

In one embodiment, the lipid vehicle is selected from the group consisting of Captex 300, Tween 85, Cremophor EL, Maisine 35-1, Maisine CC, Capmul MCM, maize oil, and any combination thereof. In one embodiment, the lipid vehicle is an oil. In one embodiment, the lipid vehicle is an oil mixture. In one embodiment, the oil mixture comprises at least two oils. In one embodiment, the oil is selected from the group consisting of Captex 300, Tween 85, Cremophor EL, Maisine 35-1, Maisine CC, Capmul MCM, maize oil, and any combination thereof.

In one embodiment, the oil mixture is 10:90 v/v oil mixture. In one embodiment, the oil mixture is 20:80 v/v mixture. In one embodiment, the oil mixture is 30:70 v/v oil mixture. In one embodiment, the oil mixture is 40:60 v/v oil mixture. In one embodiment, the oil mixture is 42:58 v/v oil mixture. In one embodiment, the oil mixture is 50:50 v/v oil mixture. In one embodiment, the oil mixture is 55:45 v/v oil mixture. In one embodiment, the oil mixture is 60:40 v/v oil mixture. In one embodiment, the oil mixture is 70:30 v/v oil mixture. In one embodiment, the oil mixture is 80:20 v/v oil mixture. In one embodiment, the oil mixture is 90:10 v/v oil mixture.

In one embodiment, the stabilizer is selected from the group consisting of Pharmacoat 603, SLS, Nisso HPC-SSL, Kolliphor, PVP K30, PVP VA 64, and any combination thereof. In one embodiment, the stabilizer is an aqueous solution.

In one embodiment, the polymer is selected from the group consisting of HPMC-AS-MG, HPMC-AS-LG, HPMC-AS-HG, HPMC HPMC-P-55S, HPMC-P-50, methyl cellulose, HEC, HPC, Eudragit L100, Eudragit E100, PEO 100K, PEG6000, PVP VA64, PVP K30, TPGS, Kollicoat IR, Carbopol 980NF, Povocoat MP, Soluplus, Sureteric, Pluronic F-68, and any combination thereof.

In one embodiment, the antioxidant is selected from the group consisting of Vitamin A, Vitamin C, Vitamin E, Coenzyme Q10, manganese, iodide, melatonin, alpha-carotene, astaxanthin, beta-carotene, canthaxanthin, cryptoxanthin, lutein, lycopene, zeaxanthin, polyphenol antioxidant, flavonoid, flavones, apigenin, luteolin, langeritin, flavonol, isorhammetin, kaempferol, myricetin, proanthocyanidinm quercetin, flavanone, eriodictyol, hesperetin, naringenin, flavanol, catechin, gallocatechin, gallate esters, epicatechin, epigallocatechin, theaflavin, thearubigin, isoflavone phytoestrogen, daidzein, genistein, glycitein, stilbenoid, resveratrol, pterostilbene, anthocyanin, cyanidin, delphinidin, malvidin, pelargonidin, peonidin, petunidin, chicoric acid, caffeic acid, chlorogenic acid, ferulic acid, cinnamic acid, ellagic acid, ellagitannin, gallic acid, gallotannin, rosmarinic acid, salicylic acid, curcumin, flavonolignan, silymarin, xanthone, eugenol, capsaicin, bilirubin, citric acid, oxalic acid, phytic acid, n-acetylcysteine, R-alpha-lipoic acid, and any combination thereof.

In one embodiment, the cannabidiol derivative or formulation thereof solubilized in a pharmaceutical vehicle has a solubility range of 0.001 mg/mL-10.0 g/mL. In one embodiment, the cannabidiol derivative or formulation thereof has a solubility of 0.001 mg/mL. In one embodiment, the cannabidiol derivative or formulation thereof has a solubility of 0.005 mg/mL. In one embodiment, the cannabidiol derivative or formulation thereof has a solubility of 0.006 mg/mL. In one embodiment, the cannabidiol derivative or formulation thereof has a solubility of 0.008 mg/mL. In one embodiment, the cannabidiol derivative or formulation thereof has a solubility of 0.01 mg/mL. In one embodiment, the cannabidiol derivative or formulation thereof has a solubility of 0.03 mg/mL. In one embodiment, the cannabidiol derivative or formulation thereof has a solubility of 0.06 mg/mL. In one embodiment, the cannabidiol derivative or formulation thereof has a solubility of 1.0 mg/mL. In one embodiment, the cannabidiol derivative or formulation thereof has a solubility of 2.0 mg/mL. In one embodiment, die cannabidiol derivative or formulation thereof has a solubility of 2.5 mg/mL. In one embodiment, the cannabidiol derivative or formulation thereof has a solubility of 6.1 mg/mL. In one embodiment, the eannabidiol derivative or formulation thereof has a solubility of 10.0 mg/mL. In one embodiment, the eannabidiol derivative or formulation thereof has a solubility of 10.2 mg/mL. In one embodiment, the eannabidiol derivative or formulation thereof has a solubility of 100.0 mg/mL. In one embodiment, the eannabidiol derivative or formulation thereof has a solubility of 250.0 mg/mL. In one embodiment, the eannabidiol derivative or formulation thereof has a solubility of 500.0 mg/mL. In one embodiment, the eannabidiol derivative or formulation thereof has a solubility of 750.0 mg/mL. In one embodiment, the eannabidiol derivative or formulation thereof has a solubility of 1.0 g/mL. In one embodiment, the eannabidiol derivative or formulation thereof has a solubility of 1.5 g/mL. In one embodiment, the eannabidiol derivative or formulation thereof has a solubility of 5.0 g/mL. In one embodiment, the eannabidiol derivative or formulation thereof has a solubility of 8.0 g/mL. In one embodiment, the eannabidiol derivative or formulation thereof has a solubility of 10.0 g/mL.

While the compounds of Formula I-X arc CB₂ receptor ligands, they also have neuroprotective properties. Thus, the compositions and formulations comprising a compound of Formula I-X are useful in treating neurological disorders including but not limited to stroke, migraine, cluster headaches. The compositions and formulations disclosed herein are also effective in treating certain chronic degenerative diseases that are characterized by gradual selective neuronal loss. In this connection, the present compositions and formulations are effective in the treatment of Parkinson's disease. Alzheimer's disease, amyotrophic lateral sclerosis. Huntington's chorea, and prison-associated neurodegeneration. Neuroproteciion conferred by CB₂ receptor agonists could also be effective in protection and/or treatment of neurotoxic agents, such as nerve gas, as well as other insults to brain or nervous tissue by way of chemical or biological agents.

By virtue of their analgesic properties it will be recognized that the compositions and formulations according to the present invention will be useful in treating pain including peripheral, visceral, neuropathic, inflammatory and referred pain. The compositions and formulations disclosed herein ate also effective in the treatment of muscle spasm and tremor.

The pharmaceutical compositions and formulations described herein can be administered to a subject per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Fasten, Pa., 18th edition, 1990.

Suitable routes of administration may, for example, include topical, oral rectal, transmucosal, or intestinal administration; parenteral delivery, including intramuscular, subcutaneous, intravenous, intramedullary injections, as well as intrathecal, direct intraventricular, intrapcritoneal, intranasal, or intraocular injections.

Alternatively, one may administer the compound in a local rather than systemic manner, for example, via injection of the compound directly into the area of pain, often in a depot or sustained release formulation. Furthermore, one may administer the drug in a targeted drug delivery system, for example, in a liposome coated with a tissue-specific antibody. The liposomes will be targeted to and taken up selectively by the organ.

The pharmaceutical compositions and formulations disclosed herein may be manufactured in a manner that is itself known, e.g., by means of conventional mixing, dissolving, granulating, dragee-making, levigating, emulsifying, encapsulating, entrapping or tabletting processes.

Pharmaceutical compositions and formulations for use in accordance with the present disclosure thus may be formulated in a conventional manner using one or more physiologically acceptable carriers comprising excipients and auxiliaries, which facilitate processing of the active compounds into preparations, which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

For injection, the agents disclosed herein may be formulated in aqueous solutions, preferably in physiologically compatible buffers such as Hank's solution. Ringer's solution, or physiological saline buffer. For transmucosal administration, penetrants appropriate to the barrier to be permeated are used in the formulation. Such penetrants are generally known in the art.

For oral administration, either solid or fluid unit dosage forms can be prepared. For preparing solid compositions such as tablets, the compound of Formula (I) or derivatives thereof, disclosed above herein, is mixed into formulations with conventional ingredients such as talc, magnesium stearate, dicalcium phosphate, magnesium aluminum silicate, calcium sulfate, starch, lactose, acacia, methylcellulose, and functionally similar materials as pharmaceutical diluents or carriers. For oral administration, the compounds can be also formulated readily by combining the active compounds with pharmaceutically acceptable carriers well known in the art. Such carriers enable the compounds disclosed herein to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination disclosed herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Capsules are prepared by mixing the compound with an inert pharmaceutical diluent, and filling the mixture into a hard gelatin capsule of appropriate size. Soft gelatin capsules are prepared by machine encapsulation of slurry of the compound with an acceptable vegetable oil, light liquid petrolatum or other inert oil. Fluid unit dosage forms for oral administration such as syrups, elixirs and suspensions can be prepared. The water-soluble forms can be dissolved in an aqueous vehicle together with sugar, aromatic flavoring agents and preservatives to form syrup. An elixir is prepared by using a hydro alcoholic (e.g., ethanol) vehicle with suitable sweeteners such as sugar and saccharin, together with an aromatic flavoring agent. Suspensions can be prepared with an aqueous vehicle with the aid of a suspending agent such as acacia, tragacanth, methylcellulose and the like.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Starch microspheres can be prepared by adding a warm aqueous starch solution, e.g., of potato starch, to a heated solution of polyethylene glycol in water with stirring to form an emulsion. When the two-phase system has formed (with the starch solution as the inner phase) the mixture is then cooled to room temperature under continued stirring whereupon the inner phase is converted into gel particles. These particles are then filtered off at room temperature and slurred in a solvent such as ethanol, after which the particles are again filtered off and laid to dry in air. The micro spheres can be hardened by well-known cross linking procedures such as heat treatment or by using chemical cross-linking agents. Suitable agents include dialdehydes, including glyoxal, malondialdehyde, succinic aldehyde, adipaldehyde, glutaraldehyde and phthalaldehyde, diketones such as butadione, epichlorohydrin, polyphosphate, and borate. Dialdehydes are used to crosslink proteins such as albumin by interaction with amino groups, and diketones form schiff bases with amino groups. Epichlorohydrin activates compounds with nucleophiles such as amino or hydroxyl to an epoxide derivative.

Pharmaceutical preparations, which can be used orally, include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers and or antioxidants may be added. All formulations for oral administration should be in dosages suitable for such administration.

For buccal administration, the compositions may take the form of tablets or lozenges formulated in conventional manner.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Slow or extended-release delivery systems, including any of a number biopolymers (biological-based systems), systems employing liposomes, colloids, resins, and other polymeric delivery systems or compartmentalized reservoirs, can be utilized with the compositions described herein to provide a continuous or long term source of therapeutic compound. Such slow release systems are applicable to formulations for delivery via topical, intraocular, oral, and parenteral routes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances, which increase the viscosity of the suspension, such as sodium carboxymethyl cellulose, sorbitol, or dextran. Optionally, the suspension may also contain suitable stabilizers or agents, which increase the solubility of the compounds to allow for the preparation of highly, concentrated solutions.

Alternatively, the active ingredient may be in powder form for constitution with a suitable vehicle, e.g., sterile pyrogen-free water, before use.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds disclosed herein is a co-solvent system comprising benzyl alcohol, a nonpolar surfactant, a water-miscible organic polymer, and an aqueous phase. A common co-solvent system used is a co-solvent system, comprising a solution of 3% w/v benzyl alcohol, 8% w/v of the nonpolar surfactant. Polysorbate 80, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of Polysorbate 80; the fraction size of polyethylene glycol may be varied; other biocompatible polymers may replace polyethylene glycol, e.g., polyvinyl pyrrolidone; and other sugars or polysaccharides may be used.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for stabilization may be employed.

Many of the compounds used in the pharmaceutical combinations disclosed herein may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to he more soluble in aqueous or other protonic solvents than are the corresponding free acids or base forms.

Pharmaceutical compositions suitable for use in the methods disclosed herein include compositions where the active ingredients arc contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The exact formulation, route of administration and dosage for the pharmaceutical compositions disclosed herein can be chosen by the individual physician in view of the patient's condition. (See e.g., Fingl et al. 1975, in “The Pharmacological Basis of Therapeutics”, Ch. 1 p. 1). Typically, the dose about the composition administered to the patient can be from about 0.5 to 1000 mg/kg of the patient's body weight, or 1 to 500 mg/kg, or 10 to 500 mg/kg, or 50 to 100 mg/kg of the patient's body weight. The dosage may be a single one or a series of two or more given in the course of one or more days, as is needed by the patient. Note that for almost all of the specific compounds mentioned in the present disclosure, human dosages for treatment of at least some condition have been established. Thus, in most instances, the methods disclosed herein will use those same dosages, or dosages that are between about 0.1% and 500%, or between about 25% and 250%, or between 50% and 100% of the established human dosage. Where no human dosage is established, as w ill be the case for newly discovered pharmaceutical compounds, a suitable human dosage can be interred from ED50 or ID50 values, or other appropriate values derived from in vitro or in vivo studies, as qualified by toxicity studies and efficacy studies in animals.

Although the exact dosage will be determined on a drug-by-drug basis, in most cases, some generalizations regarding the dosage can be made. The daily dosage regimen for an adult human patient may be. for example, an oral dose of between 0.1 mg and 2000 mg of each ingredient, preferably between 1 mg and 250 mg, e.g., 5 to 200 mg or an intravenous, subcutaneous, or intramuscular dose of each ingredient between 0.01 mg and 500 mg, preferably between 0.1 mg and 60 mg, e.g., 0.1 to 40 mg of each ingredient of the pharmaceutical compositions disclosed herein or a pharmaceutically acceptable salt thereof calculated as the free base, the composition being administered 1 to 4 times per day. Alternatively, the compositions disclosed herein may be administered by continuous intravenous infusion, preferably at a dose of each ingredient up to 400 mg per day. Thus, the total daily dosage by oral administration of each ingredient will typically be in the range 1 to 2000 mg and the total daily dosage by parenteral administration will typically be in the range 0.1 to 500 mg. Suitably the compounds will be administered for a period of continuous therapy, for example for a week or more, or for months or years.

Dosage amount and interval may be adjusted individually to provide plasma levels of the active moiety, which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen, which maintains plasma levels above the MEC for 10-90% of the time, preferably between 30-90% and most preferably between 50-90%.

In cases of local administration or selective uptake, the effective local concentration of the drag may not be related to plasma concentration.

The amount of composition administered will of course, be dependent on the subject being treated on the subject's weight, the severity of the affliction, the manner of administration and the judgment of the prescribing physician.

The pharmaceutical compositions and formulations may be prepared with pharmaceutically acceptable excipients, which may be a carrier or a diluent, as a way of example. Such compositions can be in the form of a capsule, sachet, paper or other container. In making the compositions, conventional techniques tor the preparation of pharmaceutical compositions may be used. For example, the compounds of Formula (I) disclosed above herein may be mixed with a carrier, or diluted by a carrier, or enclosed within a carrier that may be in the form of an ampoule, capsule, sachet, paper, or other container. When the carrier serves as a diluent, it may be solid, semi-solid, or liquid material that acts as a vehicle, excipient, or medium for the active compound. The compounds of Formula (I) and compositions comprising the same, for use as described above herein can be adsorbed on a granular solid container for example in a sachet. Some examples of suitable carriers are water, salt solutions, alcohols, polyethylene glycols, polyhydroxyethoxylated castor oil, peanut oil, olive oil, lactose, terra alba, sucrose, cyclodextrin, amylose, magnesium stearate, talc, gelatin, agar, pectin, acacia, stearic acid or lower alkyl ethers of cellulose, silicic acid, fatty acids, fatty acid amines, fatty acid mono glycerides and diglycerides, pentaerythritol fatty acid esters, polyoxyethylene, hydroxymethylcellulose, and polyvinylpyrrolidone. Similarly, the carrier or diluent may include any sustained release material known in the art, such as glyceryl monostearate or glyceryl distearate, alone or mixed with a wax. Said compositions may also include wetting agents, emulsifying and suspending agents, preserving agents, sweetening agents or flavoring agents. The compositions for use in the treatment of conditions or diseases responsive to the modulation of the CB₂ receptor activity, described in present invention may be formulated so as to provide quick, sustained, or delayed release of the compounds of Formula (I) disclosed herein after administration to the patient by employing procedures well known in the art.

The pharmaceutical compositions and formulations can be sterilized and mixed, if desired, with auxiliary agents, emulsifiers, salt for influencing osmotic pressure, buffers and/or coloring substances and the like, which do not deleteriously react with the compounds disclosed above herein.

The pharmaceutical compositions and formulations may be prepared, packaged, or sold in the form of a sterile injectable aqueous or oily suspension or solution. This suspension or solution may be formulated according to the known an, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3 butane diol, for example. Other acceptable diluents and solvents include, but are not limited to, Ringer's solution, isotonic sodium chloride solution, and fixed oils such as synthetic mono or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

The compositions of the invention may, if desired, be presented in a pack or dispenser device, which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound disclosed herein formulated in a compatible pharmaceutical carrier may also be prepared, placed in an appropriate container, and labeled for treatment of an indicated condition.

### Treatment

The invention also relates, in part, to a method of treating a condition or disease associated with demyelination in a subject in need thereof. In one embodiment, the method comprises administering to the subject in need thereof a therapeutically effective amount of at least one cannabidiol derivative or a formulation thereof. In one aspect of the invention, the method of treating a condition or disease associated with demyelination comprises remyclination. The invention further relates, in part, to a method of remyelination in a subject in need thereof. In one aspect of the invention, the method comprises administering to the subject a therapeutically effective amount of at least one cannabidiol derivative or a formulation thereof. In one embodiment, tire subject has a condition or disease associated with demyelination. In one embodiment, the subject has a condition or disease responsive to the modulation of the CB₂ receptor activity. In one embodiment, the subject has a condition or disease associated with demyelination and a condition or disease responsive to the modulation of the CB₂ receptor activity. The present invention also relates, in part, to a method of treating demyelination diseases.

In some embodiments, the condition or disease associated with demyelination is selected from the group consisting of autoimmune disease, demyelinating disease, inflammatory-related disorder, and any combination thereof. In one embodiment, the condition or disease associated with demyelination is selected from the group consisting SSc, myelinodastic disorder, analgesia, acute and chronic pain, inflammatory pain, post-operative pain, neuropathic pain, muscle relaxation, immunosuppression, as anti-inflammatory agents, for allergies, glaucoma, bronchodilation, neuroprotection, osteoporosis and disorders of the skeletal system, cancer, neurodegenerative disorders including but not limited to Alzheimer's disease. Parkinson's disease (PD). and Huntington's disease, MS, muscle spasticity, tremor, fibromyalgia, lupus, rheumatoid arthritis, myasthenia gravis, other autoimmune disorders, irritable bowel syndrome, interstitial cystitis, migraine, pruritis, eczema, seborrhea, psoriasis, shingles, cerebral ischemia, cerebral apoplexy, craniocerebral trauma, stroke, spinal cord injury, liver cirrhosis, atherosclerosis, as an anti-tussive, asthma, nausea, emesis, gastric ulcers, neuromyelitis optica, central nervous system neuropathy, central pontine myelinolysis, myelopathy, leukoencephalopathy, leukodystrophy, peripheral neuropathy, Guillain-Barre syndrome, anti-MAG peripheral neuropathy, Charcot-Marie-Tooth disease, progressive inflammatory neuropathy, amyotrophic lateral sclerosis (ALS), and any combination thereof.

In one embodiment, the non-reactive synthetic cannabidiol derivative modulates remyelination. In one embodiment, the non-reactive synthetic cannabidiol derivative induces remyclination. In one embodiment, the non-reactive synthetic cannabidiol derivative enhances re-myelination. In one embodiment, the non-reactive synthetic cannabidiol derivative modulates demyelination. In one embodiment, the non-reactive synthetic cannabidiol derivative prevents demyelination. In one embodiment, the non-reactive synthetic cannabidiol derivative reduces demyelination. In one embodiment, the non-reactive synthetic cannabidiol derivative accelerates demyelination. In one embodiment, the non-reactive synthetic cannabidiol derivative terminates demyelination. In one embodiment, the non-reactive synthetic cannabidiol derivative modulates neuroinflammation. In one embodiment, the non-reactive synthetic cannabidiol derivative alleviates neuroinflammation. In one embodiment, the non-reactive synthetic cannabidiol derivative modulates microgliosis. In one embodiment, the non-reactive synthetic cannabidiol derivative prevents microgliosis. In one embodiment, the non-reactive synthetic cannabidiol derivative alleviates microgliosis. In. one embodiment, the non-reactive synthetic cannabidiol derivative modulates astrogliosis. In one embodiment, the non-reactive synthetic cannabidiol derivative prevents astrogliosis. In one embodiment, the non-reactive synthetic cannabidiol derivative alleviates astrogliosis.

In one embodiment, the non-reactive synthetic cannabidiol derivative modulates a gene expression. In one embodiment, the non-reactive synthetic cannabidiol derivative prevents a gene expression. In one embodiment, the non-reactive synthetic cannabidiol derivative reduces a gene expression. In one embodiment, the non-reactive synthetic cannabidiol derivative enhances a gene expression.

In some embodiments, the non-reactive synthetic cannabidiol derivative modulates a gene expression selected from the group consisting of a gene associated with MS pathophysiology, a gene associated with oligodendrocyte function, a gene associated with downregulation in EAE, a gene associated with expression of Olig2, and any combination thereof. In one embodiment, the non-reactive synthetic cannabidiol derivative modulates an expression of Teneurin. In one embodiment, the non-reactive synthetic cannabidiol derivative modulates an expression of Teneurin 4 (Tenm 4). In one embodiment, the non-reactive synthetic cannabidiol derivative enhances an expression of Tenm 4. In one embodiment, the non-reactive synthetic cannabidiol derivative normalizes an expression of Tenm 4. In one embodiment, the non-reactive synthetic cannabidiol derivative modulates an expression of Olig2. In one embodiment, the non-reactive synthetic cannabidiol derivative restores an expression of Olig2. In one embodiment, the non-reactive synthetic cannabidioi derivative enhances an expression of Olig2. In one embodiment, the non-reactive synthetic cannabidioi derivative modulates an expression of glutathione S-transferase pi (GSTpi). In one embodiment the non-reactive synthetic cannabidiol derivative enhances an expression of GSTpi. In one embodiment, the non-reactive synthetic cannabidiol derivative restores an expression of GSTpi.

In one embodiment, the non-reactive synthetic cannabidiol derivative is effective for the attenuation of demyelination in a subject. By “attenuation of demyelination” it is meant that the amount of demyelination in the subject as a result of the disease or as a symptom of the disease is reduced when compared to otherwise same conditions and or the amount of remyelination in the subject is increased when compared to otherwise same conditions. By “reduced” it is meant any measurable or detectable reduction in the amount of demyelination or in any symptom of the demyelination disease that is attributable to demyelination. Likewise, the term “increased” means any measurable or detectable increase in the amount of remyelination which will also manifest as a reduction in any symptom of the demyelination disease that is attributable to demyelination. In an embodiment of the invention, attenuation of demyelination in a subject is as compared to a control. Symptoms attributable to demyelination will vary depending on the disease but may include, tor example but not limited to, neurological deficits, such as cognitive impairment (including memory, attention, conceptualization and problem-solving skills) and information processing; paresthesias in one or more extremities, in the trunk, or on one side of the face; weakness or clumsiness of a leg or hand; or visual disturbances, e.g., partial blindness and pain in one eye (retrobulbar optic neuritis), dimness of vision, or scotomas. The ability of a compound to attenuate demyelinaiton may be detected or measured using assays known in the art, for example, the cuprizone-induced demyelination models described herein.

In one embodiment the demyelination disease is any disease or condition that results in damage to the protective covering (myelin sheath) that surrounds nerves in the brain and spinal cord. In a further embodiment of the invention, the demyelination disease is selected from multiple sclerosis, transverse myelitis. Gutllain Barre syndrome, progressive multifocal leukoencephalopathy, transverse myelitis, phenylketonuria and other aminoacidurias. Tay-Sachs disease, Niemann-Piek disease, Gaucher's diseases, Hurler's syndrome. Krabbe's disease and other leukodystrophies, acute disseminated encephalomyelitis (postinfectious encephalomyelitis, adrenoleukodystrophy, adrenomyeloneuropathy, optic neuritis. Devie disease (neuromyelitis optica), Leber's hereditary optic atrophy and related mitochondrial disorders and HTLV-associated myelopathy or the demyelination disease is a result of local injury, ischemia, toxic agents, or metabolic disorders. In one embodiment, the demyelination disease is multiple sclerosis.

CB₂ modulators (i.e., agonists, partial agonists, antagonists, or inverse agonists) have therapeutic utility for analgesia, acute and chronic pain, inflammatory pain, post-operative pain, neuropathic pain, muscle relaxation, immunosuppression, as anti-inflammatory agents, for allergies, glaucoma, bronchodilation, neuroprotection, osteoporosis and disorders of the skeletal system, cancer, neurodegenerative disorders including but not limited to Alzheimer's disease, Parkinson's disease (PD), and Huntington's disease, multiple sclerosis (MS), muscle spasticity, tremor, fibromyalgia, lupus, rheumatoid arthritis, myasthenia gravis, other autoimmune disorders, irritable bowel syndrome, interstitial cystitis, migraine, pruritis, eczema, sebhorea, psoriasis, shingles, cerebral ischemia, cerebral apoplexy, craniocerebral trauma, stroke, spinal cord injury, liver cirrhosis, atherosclerosis, as an anti-tussive, asthma, nausea, emesis, gastric ulcers, systemic sclerosis, myelinoclastic disorder, neuromyelitis optica, central nervous system neuropathy, central pontine myelinolysts, myelopathy, leukoencephalopathy, leukodystrophy, peripheral neuropathy, Guillain-Barre syndrome, anti-MAG peripheral neuropathy, Charcot-Marie-Tooth disease, progressive inflammatory neuropathy, amyotrophic lateral sclerosis (ALS), and diarrhea.

Thus, in one aspect, the present invention further relates to a method of treating a disease or condition responsive to a modulation of CB₂ receptor activity in a subject, the method comprising identifying a subject in need thereof, and administering to the subject a therapeutically effective amount of a cannabidiol derivative or formulation thereof. In one aspect, the present invention relates to new drug candidates comprising chemically stable, nonpsychotropic aminoquinoid chemically derived from synthetic or natural cannabidiol (CBD) through oxidation and animation. In one embodiment, a non-reactive synthetic cannabidiol derivative has a novel MOA by targeting complementary signaling pathways that alleviate neuroinflammation and favor neuroprotection, prevent axonal damage, preserve myelin structure, and potentially promote remyelination. In one embodiment, the non-reactive synthetic cannabidiol derivative is a modulator of CB₂ receptor signaling. In one embodiment, the non-reactive synthetic cannabidiol derivative is a modulator of PPARγ and CB₂ receptor signaling. In one embodiment, the non reactive synthetic cannabidiol derivative is a dual modulator of PPARγ and CB₂ receptor signaling, and it activates the HIF pathway by stabilizing HIP-1α and upregulates the expression of several associated (actors that include Erythropoietin (EPO) and Vascular Endothelial Growth Factor A (VEGFA). In one embodiment, the non-reactive synthetic cannabidiol derivative reduces neuroinflammation presumably by acting on PPARγ/CB₂ receptors, in conjunction with enhanced neuroprotection and potential remyelination through the HIF pathway.

In one embodiment, the non-reactive synthetic cannabidiol derivative modulates the activity of a CB₂. In one embodiment, the non-reactive synthetic cannabidiol derivative preferentially binds to CB₂ receptor as compared to CB₁. Therefore, in these embodiments, the non-reactive synthetic cannabidiol derivative is selective for CB₂. In one embodiment, the amine group of non-reactive synthetic cannabidiol derivative enhances its binding to the CB₂. In one embodiment, the amine group of non-reactive synthetic cannabidiol derivative selectively binds the CB₂ receptor over the CB₁ receptor. In one embodiment, the CB₂ receptor activity is modulated in vitro, whereas in other embodiments, the CB₂ receptor activity is modulated in vivo.

In one embodiment, the cannabidiol derivative or formulation thereof is administered in combination with another therapeutic agent. In one embodiment, the cannabidiol derivative or formulation thereof is administered orally. In one embodiment, the cannabidiol derivative or formulation thereof is administered topically. In one embodiment, the cannabidiol derivative or formulation thereof is administered using rectal administration. In one embodiment, the cannabidiol derivative or formulation thereof is administered using transmucosal administration. In one embodiment, the cannabidiol derivative or formulation thereof is administered using intestinal administration. In one embodiment, the cannabidiol derivative or formulation thereof is administered using parenteral delivery. In one embodiment, the cannabidiol derivative or formulation thereof is administered using intramuscular injection. In one embodiment, the cannabidiol derivative or formulation thereof is administered using subcutaneous injection. In one embodiment, the cannabidiol derivative or formulation thereof is administered using intravenous injection. In one embodiment, the cannabidiol derivative or formulation thereof is administered using intramedullary injection. In one embodiment, the cannabidiol derivative or formulation thereof is administered using intrathecal injection. In one embodiment, the cannabidiol derivative or formulation thereof is administered using direct intraventricular injection. In one embodiment, the cannabidiol derivative or formulation thereof is administered using intraperitoneal injection. In one embodiment, the cannabidiol derivative or formulation thereof is administered using intranasal Injection. In one embodiment, the cannabidiol derivative or formulation thereof is administered using intraocular injection.

In one embodiment, the cannabidiol derivative or formulation thereof is administered with food or drink.

In one embodiment, the condition or disease responsive to the modulation of the CB₂ receptor activity is selected from the group consisting of autoimmune disease, demyelinating disease, inflammatory-related disorder, and any combination thereof. In one embodiment, the condition or disease responsive to the modulation of the CB₂ receptor activity is selected from the group consisting SSc, myelinoclastic disorder, analgesia, acute and chronic pain, inflammatory pain, post-operative pain, neuropathic pain, muscle relaxation, immunosuppression, as anti-inflammatory agents, for allergies, glaucoma, bronchodilation, neuroprotection, osteoporosis and disorders of the skeletal system, cancer, neurodegenerative disorders including but not limited to Alzheimer's disease, Parkinson's disease (PD), and Huntington's disease, MS, muscle spasticity, tremor, fibromyalgia, lupus, rheumatoid arthritis, myasthenia gravis, other autoimmune disorders, irritable bowel syndrome, interstitial cystitis, migraine, pruritis, eczema, sehhorea, psoriasis, shingles, cerebral ischemia, cerebral apoplexy, craniocerebral trauma, stroke, spinal cord injury, liver cirrhosis, atherosclerosis, as an anti-tussive, asthma, nausea, emesis, gastric ulcers, neuromyelitis optica, central nervous system neuropathy, central pontine myelinolysis, myelopathy, leukoencephalopathy, leukodystrophy, peripheral neuropathy, Guillain-Barre syndrome, anti-MAG peripheral neuropathy, Charcot-Marie-Tooth disease, progressive inflammatory neuropathy, amyotrophic lateral sclerosis (ALS). and any combination thereof.

It will be understood by those of skill in the art that numerous and various modifications can be made without departing from the spirit of the present disclosure. Therefore, it should be clearly understood that the forms disclosed herein are illustrative only and are not intended to limit the scope of the present disclosure.

### EXPER1MENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### Synthesis of the Compounds

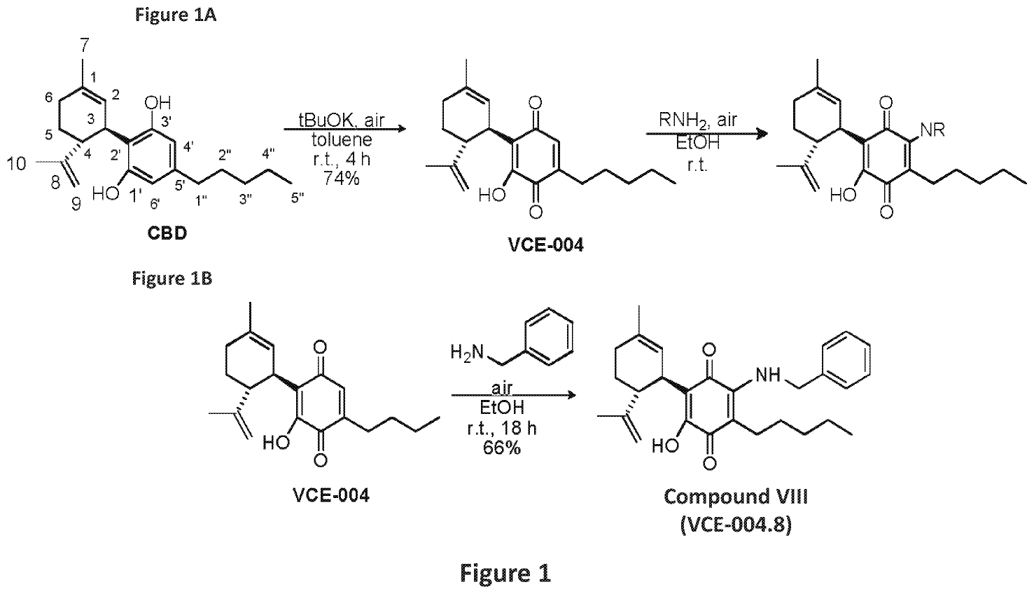

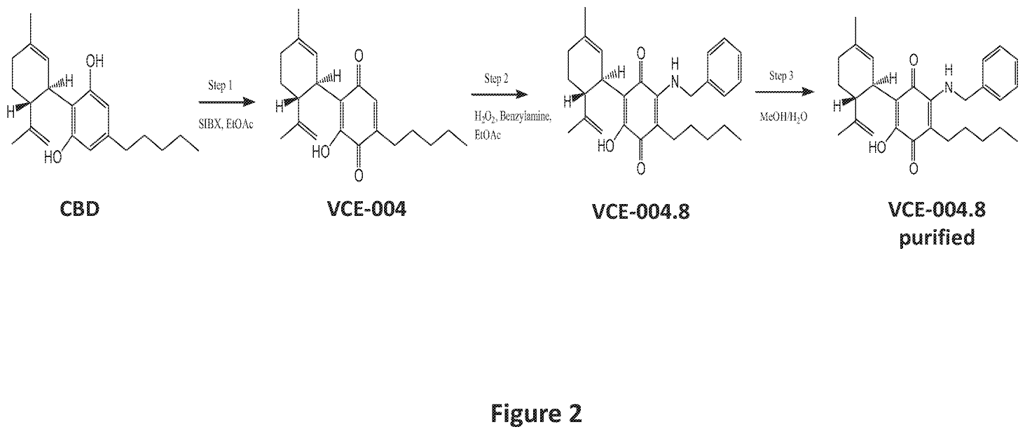

The current manufacturing process of VCE-004.8 comprises three steps as shown in FIGS. 1A-1B and 2. In short, these steps are:

Step 1: CBD is oxidized by the addition of stabilized 2-iodoxybenzoic acid (SIBX) to a solution of CBD in ethyl acetate (EtOAc). The heterogenic mixture is stirred at elevated temperature and after completion the mixture is filtered. The filtrate is washed twice with potassium carbonate (K₂CO₃) solution and once with hydrochloric acid (HCl) solution. Sodium Chloride (NaCl) (aq, sat) is added to the last washing to facilitate layer separation. The organic layer is concentrated to give VCE-004.

Step 2: A peroxide solution in water is added to a solution of VCE-004 in EtOAc. The mixture is cooled and benzylamine is added slowly. After completion of the reaction, aqueous HCl (15%) is added and the organic layer is washed several times with water. The organic layer is concentrated, and the product is precipitated from a solution of methanol and water (MeOH/H₂O). filtered and dried to produce VCE-004.8.

Step 3: VCE-004.8 is further purified by suspension in MeOH/H₂O 85:15 at elevated temperature. The resulting mixture is cooled, and the product is filtered. The solid is dried and sieved to produce VCE-004.8 purified.

The final Drug Substance is sieved, packaged in a double low-density polyethylene bag and Kraft drum, then labelled.

VCE-004.8 is a new chemical entity described in PCT-EP2014-057767. The activity of the compound is also described in PCT-EP2017-057389. PCT-EP2014-057767 and PCT-EP2017-057389 arc incorporated by reference herein in their entirety.

The aminoquinoid VCE-004.8 is a new chemical entity derived from synthetic cannabidiol (CBD). Characterization studies showed that VCE-004.8 is an anhydrous and non-solvated crystalline solid with a molecular weight of 433.6 g/mol. The melting point is 90.7° C. Structural elucidation of VCE-004.8 was performed by Infrared Spectroscopy (ATR-IR). Elemental analysis (CHN), High Resolution Electro spray Ionization Mass Spectrometry (ESI-MS), Proton Nuclear Magnetic Resonance (1H-NMR), Carbon Nuclear Magnetic Resonance (13C-NMR), other NMR techniques i.e., Distortionless Enhancement by Polarization Transfer (DEPT135), Heteronuclear Single Quantum Correlation (HSQC), Heteronuclear Multiple Bond Correlation (HMBC) and 2D studies. These structural elucidation studies are completed, and structure of the molecule has been conformed.

Analytical test methods for release and stability testing of VCE-004.8 Drug Substance were developed for identity, individual and total impurities, chromatographic purity and assay (Table 1). Potential chiral impurities were also evaluated. Since the raw material CBD is highly pure and during synthesis hardly to no enantiomeric form is obtained, the chance of chiral impurity formation during Drug Substance manufacturing is considered to be very low. Nevertheless, a chiral method was developed to evaluate the Drug Substance lots.

TABLE 1
Proposed Specifications for VCE-004.8 Drug Substance.

| Parameter | Method | Acceptance Criteria |
|---|---|---|
| Appearance | Visual evaluation | Purple powder |
| Identity | UPLC-UV | Retention time consistent with |
| | | reference standard |
| | IR-ATR | Conforms to spectrum of reference |
| | | standard |
| Sulphated Ash | Ph. Eur. 2.4.14 | ≤1.0% |
| Water content | Karl Fisher Ph. Eur. 2.2.32 | ≤2.0% |
| Residual solvents: | GC | |
| Ethyl Acetate | | ≤5000 ppm |
| Methanol | | ≤3000 ppm |
| Chromatograpic Purity | UPLC-UV | ≥97.0% |
| Individual impurities | UPLC-UV | Report value for individual impurities |
| | | impurities ≥0.05% |
| Total impurties | UPLC-UV | ≤3.0% |
| Assay | UPLC-UV | 95.0%-105.0% |
| Enantiomeric purity | Chiral LC-UV (internal method) | ≥98% |
| Microbial Quality | Ph. Eur. 2.6.12 and 2.6.13 | |
| TAMC | | 10³ CFU/g |
| TYMC | | 10² CFU/g |
| E. Coli | | Absence in 1 g |

A stress test was performed concluding that VCE-004.8 drug substance is stable for 3 days at 65° C.+/−5° C. in glass vials. Degradation of the product was observed at temperatures above 65° C. A short-term stability study at 40° C. was completed with indecisive results due to the early development stage of the impurity method.

### Example 3

### Liquid Formulation

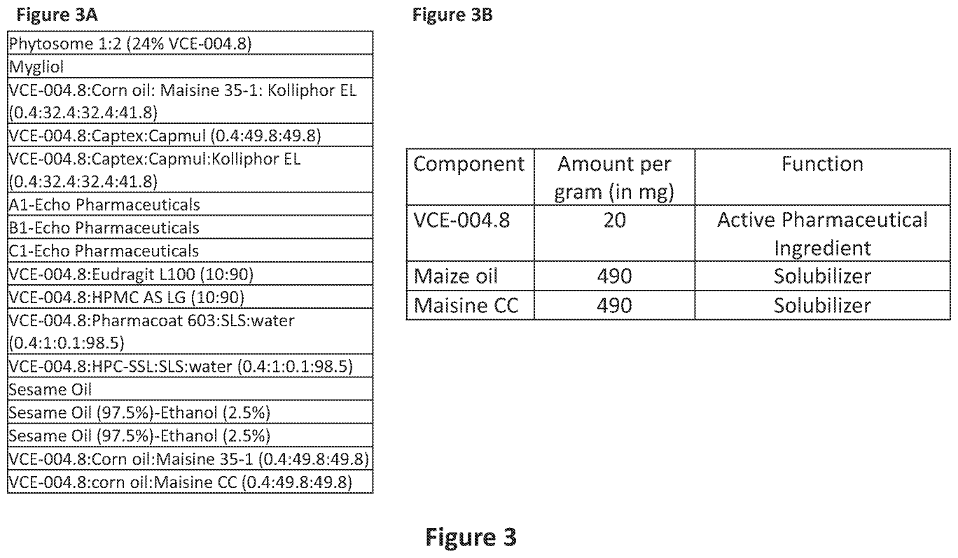

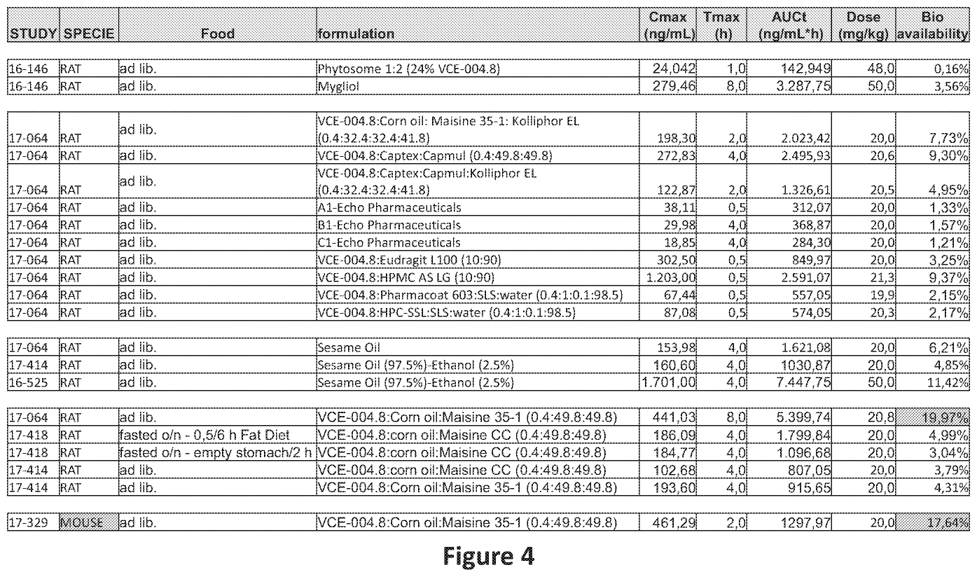

A solubility screening study showed that VCL-004.8, the active ingredient of EHP-101 Liquid (in preclinical development also known as VCE-004.8 formulation), is practically insoluble in aqueous solutions at different pH and in cyclodextrin complexes (FIGS. 3A, 3B, and 4). It is also practically insoluble in co-solvents, such as glycerol, and sparingly soluble in a co-solvent like PEG400. VCE-004.8 is slightly soluble in organic solvents like n-heptane and methanol to freely soluble in organic solvents like DMSO and DCM Based on solubility studies, short-term stability studies, and an in vivo bioavailability study in rats and mice, the composition of EHP-101 as shown in Table 2 was selected for the oral formulation

TABLE 2
Composition of Drug Product EHP-101 Liquid

| | Amount per gram | |
|---|---|---|
| Component | (in mg) | Function |
| VCE-004.8 | 20 | Active Pharmaceutical Ingredient |
| Maize oil | 490 | Solubilizer |
| Maisine CC | 490 | Solubilizer |

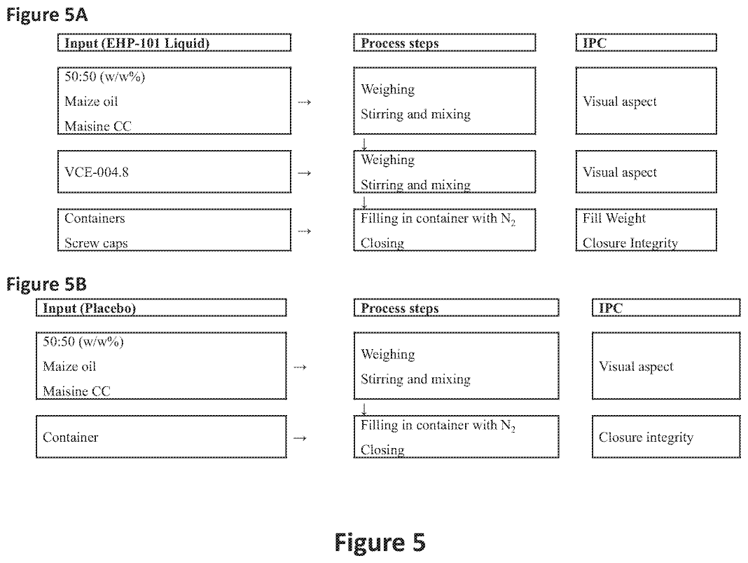

In the manufacturing flow chart of FIG. 5A, the current manufacturing process for tire GMP DP batch (20 mg/g) is described based on the experience to date for the formulation EHP-101 Liquid and Placebo/Vehicle. The process comprises the following steps:

1. Mixing Maisine CC and Maize Oil in a ratio of 50:50 v/v

2. Solubilize VCE-004.8 in the Maisine CC:Maize Oil mixture

3. Filling the DP in bulk containers with N2 blanketing.

The bulk mixture of Maisine CC and Maize Oil (50:50 v/v) vehicle will be used for Placebo in the clinical studies (FIG. 5B).

Analytical test methods developed and used for the control of FHP-101 Liquid and Placebo are summarized in Table 3. In the course of development, analytical test methods will continue to be optimized and revised.

TABLE 3
Tentative Release Specifications for Bulk EHP-101 and Bulk Placebo

| Parameter | | |
|---|---|---|
| (Bulk EPH-101 Liquid) | Test method | Acceptance limits (Release) |
| Appearance | Visual inspection | Dark purple, homogeneous, oily liquid |
| Identity | UPLC-UV | The retention time ofVCE-004.8 peak |
| | | obtained from the sample preparation is |
| | | within ±5.0% of the retention time of |
| | | VCE-004.8 peak obtained from the first |
| | | injection of reference solution 1 |
| | IR | Conforms to spectrum of reference |
| | | standard |
| Assay | UPLC-UV | 90.0-110.0% of label claim |
| Chromatographic purity | UPLC-UV | |
| Any unspecified degradant | | ≤0.30% |
| Total degradant | | ≤3.0% |
| Enantiomeric purity | LC | ≥98% |
| Microbial Purity | Ph. Eur. 2.6.12 and 2.6.13 | |
| TAMC | | 10³ CFU/g |
| TYMC | | 10² CFU/g |
| E. Coli | | Absence in 1 g |
| Parameter (Bulk Placebo) | Test method | Acceptance limits |
| Appearance | Visual inspection | Clear, slightly yellow solution |
| Identity | HPLC-UV | The drug product assay method confims |
| | | the absence of drug substance at or above |
| | | the limit of detection of the method |
| Microbial Purity | Ph. Eur. 2.6.12 and 2.6.13 | |
| TAMC | | 10³ CFU/g |
| TYMC | | 10² CFU/g |
| E. Coli | | Absence in 1 g |

Data of a 6-month stability study are available, in which formulations were included at three different concentrations, i.e., 20 mg/g, 25 mg/g and 30 mg/g. The oil formulation composition is identical to the selected composition of the formulation to be used in the clinical studies. Therefore, these formulations are representative of the formulation to be used in the clinical studies. The stability study was conducted at the following conditions: 5° C.±3° C., 25° C.±2° C./60% RH±5% RH and 40° C.±2° C./75% RH±5% RH.

The results of this study showed that the product is chemically stable tor at least 6 months at 5° C., 25° C./60% RH in amber glass bottles, without nitrogen blanketing.

### Example 4

### Formulations for Phase 1 Studies

Different concentrations of Drug Substance were tested in this lipid formulation i.e., 20 mg/g, 25 mg/g and 30 mg/g. Because the concentration of 20 mg/g remains solubilized at room temperature without additional heating or swirling, this concentration was selected to be used in the clinical studies.

EHR-101 Liquid and Placebo are filled, stored and shipped in bulk bottles.

The liquid formulation, EHP-101 Liquid, disclosed in this invention, consists of a 20 mg/g solution of VCE-004.8 in a mixture of maize oil/Maisine CC (50/50 v/v). A similar formulation (up to a concentration of 30 mg/g) has been used for in vivo nonclinical studies. The selection of the liquid oily formulation was based on the solubilization efficiency of VCE-004.8 and in vivo screening studies of the bioavailability of >20 formulation prototypes.

Manufacturing of single dose formulations will be prepared by diluting the bulk EHP-101 with the bulk vehicle. Matching placebos will be prepared by addition of a colorant to the bulk placebo. Analytical methods will be transferred in order to release the single dose formulations and matching placebo and to conduct stability studies on these formulations.

### Solubility Screening and Manufacturing of Formulation Concepts

In order to select the best formulation of VCE-004.8 for oral administration (LHP-101), two main parameters were considered: solubility and oral bioavailability.

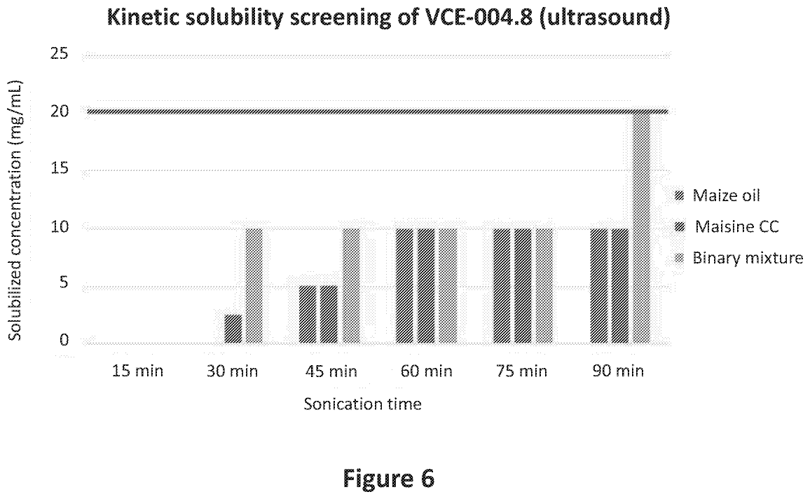

The solubility of a compound is an important factor in determining its absorption from the gastrointestinal tract and ultimately its oral bioavailability. First it was determined the solubility of VCE-004.8 in a collection of different solvents (e.g., aqueous, lipidic, organic, etc.). Additionally, a test of stability of VCE-004.8 in selected solvents was also used as a criterion for selection of the best solvents. Based on solubility studies in lipidic solvents, VCE-004.8 was shown to be more soluble in a mixture of Maisine CC: maize oil than in individual com oil or Maisine CC alone (Table 4 and FIG. 6).

TABLE 4
Solubility studies of VCE-004.8 in lipidic solvents at 25° C. and 37° C.

| Solvent | 25° C. | 37° C. |
|---|---|---|
| Maisine CC | 17.7 mg/mL | 34.5 mg/mL |
| Corn oil | 19.3 mg/mL | |
| Maisine CC/Corn oil (50:50) | 20.3 mg/mL | 35.6 mg/mL |

Selected solvents were used to manufacture several formulation concepts of VCE-004.8 which pharmacokinetic (PK) profile by oral intake was assessed.

Bioavailability is one of the principal PK properties of drugs. It is used to describe the fraction of an administered dose of unchanged drug that reaches the systemic circulation. The measurement of the amount of the drug in the plasma at periodic time intervals indirectly indicates the rate and extent at which the active pharmaceutical ingredient is absorbed from the drug product and becomes available at the site of action.

### Example 5

### Turbidimetric Aqueous Solubility

An aqueous solubility assessment for VCE-004.8 was performed at physiological temperature. VCE-004.8 (dissolved at 10 mM in DMSO) was mixed with PBS buffer pH 7.4 at 37° C. to achieve a final VCE-004.8 concentration of 1 μM and a final DMSO concentration of 0.33% v/v. Incubations were performed in PTFE (Teflon®). A parallel incubation was also performed in a polypropylene plate to assess any differences in non-specific binding between PTFE and polypropylene. For the incubations in PTFE. serial samples were then taken over a 2 hr period at 5, 15, 30, 45 and 120 min. For the incubation in polypropylene, samples were removed at 0 min and 120 min only. All samples were added immediately to two volumes of methanol in a microtiter plate cooled in dry-ice to halt chemical degradation. When sampling was complete, the sampling plate was allowed to reach room temperature. Samples were then removed for quantitative analysis of parent compound by LC-MS/MS. An internal standard was included to correct for analytical variation (nicardipine and pyrene). The percentage of parent compound remaining at each time point relative to the 0 min sample and the percentage of parent compound bound to polypropylene compared to PTFE was then calculated from LC-MS/MS peak area ratios (compound peak area/internal standard peak area). The percent of parent compound present at 0, 5, 15, 30, 45 and 120 min after initiating incubations at 37° C. was reported for the PTFE incubations, in addition, the percentage of test compound bound to polypropylene compared to PTFE was calculated. Estimated solubility range (lower and upper bound and calculated mid-range in μM) are shown in Table 5, indicating a low aqueous solubility of VCE-004.8.

TABLE 5
Estimated Precipitation Range (μM) of VCE-004.8 in
Aqueous Solubility test at 37° C. compared to nicardipine and pyrene.

| | Estimated Precipitation Range (μM) | | |
|---|---|---|---|
| | | | Calculated |
| Test Compound | Lower Bound | Upper Bound | Mid-range |
| VCE-004.8 | <1 | 6.5 | <6.5 |
| nicardipine | 10 | 30 | 20 |
| pyrene | 3 | 10 | 6.5 |

### Example 6

### Log D Determination

Lipophilicity is a key determinant of the PK behavior of drugs. It can influence distribution into tissues, absorption and the binding characteristics of a drug, as well as being an important factor in determining the solubility of a compound. Log D (distribution co-efficient) is used as a measure of lipophilicity. Determining the partition of a compound between an organic solvent (typically octanol) and aqueous buffer is one of the most common methods for determining this parameter.

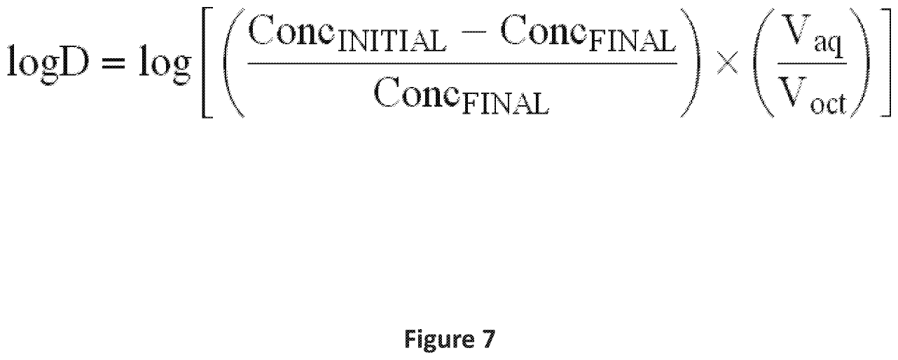

To determine log D, 0.1 M phosphate buffer pH 7.4 (saturated with octanol) was added to the vial containing VCH-004.8 and the solution mixed and sonicated for approximately 15 min. The solution was transferred to tubes, centrifuged and the supernatant is drawn off the top, leaving any solid compound in the bottom. This supernatant was then syringe filtered through 0.2 μm filters to produce the initial solution. Three vials were prepared containing values. Ketoconazole and cannabidiol (CBD) were used as control. The vials were mixed to equilibrium, then centrifuged to ensure the two phases were fully separated before the octanol was removed and the buffer samples analyzed. For the quantitative analysis, the aqueous solutions were analyzed by LC MS/MS. The amount of VCE-004.8 in each vial was quantified against a 6 points standard curve which was produced by serially diluting the initial solution. Log D was calculated using the equation, shown in FIG. 7, wherein ConcINITlAL is a concentration of compound in the initial aqueous solution, ConcFINAL is a concentration of compound in final aqueous phase, Vaq is a volume of aqueous phase, and Voct is a volume of octanol phase.

Results showed in Table 6 indicate that VCE-004.8 is a highly lipophilic compound, in the same range than the parent molecule cannabidiol (CBD).

TABLE 6
LogD7.4 Octanol of VCE-004.8 compared to CBD and ketoconazole.

| Test Compound | LogD_{7.4} Octanol |
|---|---|
| VCE-004.8 | >5 |
| CBD | 5.44 |
| ketoconazole | 3.52 |

### Example 7

### Solubility Screening

A quantitative thermodynamic solubility determination on VGE-004.8 was performed. Suspensions of VCE-004.8 were prepared in different pharmaceutical vehicles and organic solvents. The organic solvents, lipid and co-solvent vehicles consisted of pure solvent or lipid, while the cyclodextrin solutions were prepared in phosphate buffer pH 7.0. After stirring the suspensions for 24 hr at 25° C., a small aliquot of the mother liquor was taken from the suspensions for a solubility determination. The concentration of VCE-004.8 in solution was determined by HPLC analysis. The results of this solubility determination are presented in Table 1.

TABLE 7
Solubility results for VCE-004.8.

| Vehicle | Solubility (mg/mL) ⁽¹⁾ | |
|---|---|---|
| Aqueous buffers | | |
| 0.1N HCl (pH = 1.0) | <0.008 | Practically insoluble |
| 0.1N Citrate-HCl buffer pH2 | <0.008 | Practicalty insoluble |
| (pH = 3.0) | | |
| 0.1N Citrate-NaOH buffer pH5 | <0.008 | Practically insoluble |
| (pH = 5.0) | | |
| 1.1N Phosphate buffer (pH = 7.0) | <0.008 | Practically insoluble |
| 0.1N Borate-KCl—NaOH buffer | <0.008 | Practically insoluble |
| (pH = 9.0) | | |
| Organic solvents | | |
| Dichloromethane | >308 | Freely soluble |
| Chloroform | >234 | Freely soluble |
| DMSO | >199 | Freely soluble |
| Acetone | 121.9 | Freely soluble |
| Acetonitrile | 13.1 | Sparingly soluble |
| Ethanol | 10.2 | Sparingly soluble |
| Methanol | 6.1 | Slightly soluble |
| n-Heptane | 2.5 | Slightly soluble |
| Co-solvents | | |
| Transcutol P | 49.3 | Soluble |
| PEG400 | 14.0 | Sparingly soluble |
| Propylene glycol | 1.0 | Slightly soluble |
| Glycerol | <0.008 | Practically insoluble |
| Cyclodextrin complexes | | |
| Methyl-β-cyctodextrin 20% | 0.06 | Practically insoluble |
| Methyl-β-cyclodextrin 10% | 0.03 | Practically insoluble |
| HP-β-cyclodextrin 40% | 0.01 | Practically insoluble |
| Methy-γ-cyclodextrin 20% | 0.006 | Practically insoluble |
| HP-β-cyclodextrin 1 10% | <0.008 | Practically insoluble |
| HP-β-cyclodextrin 1 20% | <0.008 | Practically insoluble |
| SBE-β-cyclodextrin 10% | <0.008 | Practically insoluble |
| SBE-β-cyclodextrin 20% | <0.008 | Practically insoluble |
| α-cyclodextrin 10% | <0.008 | Practically insoluble |
| α-cyclodextrin 20% | <0.008 | Practically insoluble |
| γ-cyclodextrin 10% | <0.008 | Practically insoluble |
| γ-cyclodextrin 20% | <0.008 | Practically insoluble |
| HP-γ-cyclodextrin 10% | <0.008 | Practically insoluble |
| HP-γ-cyclodextrin 20% | <0.008 | Practically insoluble |
| 6-O-glucosyl-β-cyclodextrin 10% | <0.008 | Practically insoluble |
| 6-O-glucosyl-β-cyclodextrin 20% | <0.008 | Practically insoluble |
| Lipid vehicles | | |
| Captex 300 | 35.7 | Soluble |
| Tween 85 | 32.6 | Sparingly soluble |
| Cremophor EL | 31.0 | Sparingly soluble |
| Maisine 35-I ⁽²⁾ | 23.7 | Sparingly soluble |
| Capmul MCM ⁽²⁾ | 22.0 | Sparingly soluble |
| Corn oil | 19.3 | Sparingly soluble |

(⁽¹⁾ As defined in Ph. Eur.:1) Practically insoluble: solubility <0.1 mg/mL.; 2) Very slightly soluble solubility betweetn 0.1-1 mg/mL; 3) Slightly soluble; solubility between 1-10 mg/mL; 4) Sparingly soluble; solubility between 10-33 mg-mL; 5) Soluble: solubility between 33-100 mg/mL; 6) Freely soluble: solubility between 100-1000 mg/mL.; 7) Very soluble: solubility >1000 mg/mL; and
⁽²⁾ The solubility of Maisine 35-1 and Capmul MCM was determined at 37° C.)

These results confirm that VCE-004.8 exhibits a very low, pH independent, solubility in aqueous buffers, however, in all lipid vehicles and in most of the co-solvents the compound was found to be sparingly soluble. Moreover, the cydodextrin solubility results indicate that for methyl-β-cyclodextrin complexation with VCE-004.8 occurs, however, the use of cyclodextrms does not significantly improve solubility.

### Example 8

### Additional Assessment of Solubility and Stability in Lipidic Solvents

An additional test of solubility in lipidic solvents was performed. Accordingly, VCE-004.8 was dissolved at room temperature and stirred during a maximum of 16 hr in 6 different lipidic solvents as depicted in Table 7. Assay of the different solubility trials was performed by HPLC using the following parameters: column C150724NC0047: Kinctex, C18: 150 mm, 4.6 mm, 2.6 μM; isocratic acetonitrile: 0.2% formic acid (90:10); flow 0.35 mL/min; wavelength 314 nm; column temperature 25° C.; run time 20 min; injection volume 10 μL. Concentration 0.1 mg/ml. was considered the theoretical 100% of the technique. Results are shown in Table 8.

TABLE 8
First assessment of VCE-004.8 solubility in lipidic solvents by HPLC
(n.d. not determined).

| | VCE-004.8 | | Assay | Impurities |
|---|---|---|---|---|
| Concept | mg/ml | Solvent | (%) | (%) |
| P01 | 0.4 | Kollisolv PEG 400 | 67.93 | 35.17 |
| | | (Polyethylene glycol 400) | | |
| P02 | 0.4 | Transcutol (Diethylene | 108.98 | 5.33 |
| | | glycomonoethyl ether) | | |
| P03 | 0.4 | Kollisolv MCT 70 (Medium | 108.65 | 0.66 |
| | | chain triglycerides) | | |
| P04 | 0.4 | Labrasol (PEG-8 Caprylic/ | 101.47 | 1.40 |
| | | Capric Glycerides) | | |
| P05 | 0.4 | Labrafil M1944CS | 94.55 | 0.48 |
| | | (PEG-5 Oleate) | | |
| P06 | 0.4 | Kollisolv PG | 93.56 | 1.62 |
| | | (Propylene glycol) | | |
| P07 | 2.0 | Kollisolv MCT 70 (Medium | 91.85 | n.d. |
| | | chain triglycerides) | | |
| P08 | 2.0 | Labrasol (PEG-8 Caprylic/ | 102.73 | n.d. |
| | | Capric Gycerides) | | |
| P09 | 2.0 | Labrafil M1944CS | 92.79 | n.d. |
| | | (PEG-5 Oleate) | | |

Based on Assay and Impurities percentages, concepts P03, P04 and P05 were selected for preliminary stability studies. VCE-004.8 was also found to be soluble at the concentration of 2 mg/mL in P07, P08, and P09. None of the solutions presented any precipitate or visible solid particles. The stability studies conditions and Assay results are shown in Table 9, indicating that P03 was the best formulation based on both solubility and stability for 31 days. Consequently. Kollisolv® MCT 70-Medium chain triglycerides (also known as Miglyol® 812 or Myritol®318) was selected to assess VCE-004.8 PK, profile by oral administration in rats. A formulation of 10 mg/mL of VCE-004.8 was prepared for the PK analysis (Formulation n°1).

TABLE 9
Stability studies of VCE-004.8 formulated in Kollisolv (P04),
Labrasol (P05) and Labrafil (P05) at 0.4 mg/ml (n.d. not determined).

| Concept | Time | Temperature | Assay (%) | Impurities (%) |
|---|---|---|---|---|
| P03 | 7 days | 4° C. | 106.48 | 0.69 |
| | | 25° C. | 111.17 | 0.97 |
| | | 40° C. | 107.41 | 1.06 |
| | 14 days | 4° C. | 111.13 | n.d |
| | | 25° C. | 105.62 | n.d |
| | | 40° C. | 110.89 | n.d |
| | 31 days | 4° C. | 108.44 | n.d |
| | | 25° C. | 109.95 | n.d |
| | | 40° C. | 107.37 | n.d |
| P04 | 7 days | 4° C. | 96.87 | 1.79 |
| | | 25° C. | 98.17 | 3.63 |
| | | 40° C. | 87.42 | 10.50 |
| P05 | 7 days | 4° C. | 91.57 | 0.99 |
| | | 25° C. | 95.09 | 1.31 |
| | | 40° C. | 91.95 | 1.91 |

### Example 9

### Lipidic Formulations

Based on results showed in Table 7, ten different prototype lipid formulation concepts were developed. The composition of the lipid vehicles was chosen as such to ensure that ail classes from the lipid classification system are represented. Preparation was done as follows, 75 mg VCE-004.8 was weighed into a suitable container to which 6.75 g of excipient was added while stirring. If necessary, the excipient was heated to 45° C. in order to become liquid. Table 10 gives an overview of the ten different lipid formulation concepts that were developed.

TABLE 10
Different lipid formulation concepts.

| | Formu- | Composition (% w/w) | | | | | | | | |
|---|---|---|---|---|---|---|---|---|---|---|
| | lation | VCE- | Corn | Maisine | Captex | Capmul | Tween | Kolliphor | Transcutol | |
| No° | type | 004.8 | oil | 35-1 | 300 | MCM | 85 | EL | HP | PEG400 |
| 1 | I-LC | 1 | 49.5 | 49.5 | | | | | | |
| 2 | II-LC | 1 | 32 | 32 | | | 35 | | | |
| 3 | IIIA-LC | 1 | 32 | 32 | | | | 35 | | |
| 4 | I-MC | 1 | | | 49.5 | 49.5 | | | | |
| 5 | II-MC | 1 | | | 32 | 32 | 35 | | | |
| 6 | IIIA-MC | 1 | | | 32 | 32 | | 35 | | |
| 7 | IIIB-MC | 1 | | | | 25 | | 49 | 25 | |
| 8 | IV | 1 | | | | | | 49.5 | 49.5 | |
| 9 | IV | 1 | | | | | | | 99 | |
| 10 | IV | 1 | | | | | | | | 99 |

For each developed concept, a sample was stored at 5° C. and 25° C./60% RH for 4 weeks. Afterwards, stability was assessed by HPLC (Table 11). All concepts, except concept 8 and 10, showed an acceptable assay at Time 0 (T0). After 4 weeks of storage (T4W) at 25° C./60% RH, concepts 2, 3, 5, 6, 7 and 9 show a significant decrease in assay (5-10%).

TABLE 11
Stability results for assay (% label claim) of VCE-004.8 in different lipidic
formulations ((1) T0 is an approximately 2.5 weeks after preparation,
stored at 5° C.; and (2) Not tested at T4W, as already failing at T0).

| | Time | Concept | | | | | | | | | |
|---|---|---|---|---|---|---|---|---|---|---|---|
| Storage | point | 1 | 2 | 3 | 4 | 5 | 6 | 7 | 8 | 9 | 10 |
| NA | T0⁽¹⁾ | 94.6 | 99.4 | 104.2 | 98.3 | 102.8 | 99.7 | 98.3 | 86.0 | 97.6 | 89.0 |
| 25° C./60% RH | T4W | 103.2 | 92.8 | 93.3 | 94.1 | 85.2 | 89.9 | 79.6 | —⁽²⁾ | 93.5 | —⁽²⁾ |

For PK supplies, lipid formulation concepts 1, 3, 4 and 6 were selected for PK assessment (Formulations n°2, 3, 4, 5 respectively). Formulation n°2 and 3 were freshly prepared as follows, 350 mg VCE-004.8 was weighed into a suitable container to which 34.65 g of excipient was added while stirring to obtain a concentration of 10 mg/g. If necessary, the excipient was heated to 45° C. in order to become liquid. Concentration was adjusted from 10 mg/g to 4 mg/g. Therefore, three vials of each Formulations no°2 and 3 were pooled by magnetic stirring, after which each formulation was diluted 2.5 times with the respective excipient mixture. If necessary, the excipients were heated to 45° C. in order to become liquid. On the other hand* Formulation n°4 and 5 were freshly prepared as follows, 140 mg VCE-004.8 was weighed into a suitable container to which 34.86 g of excipient was added while stirring to obtain a concentration of 4 mg/g.

### Example 10

### Sesame Oil

In the newly approved drug Sativex®, CBD has been formulated at a concentration of 100 mg/mL in an oral solution that includes dehydrated alcohol, sesame seed oil, strawberry flavor, and sucralose. Since CBD is the parent molecule of VCE-004.8, sesame oil was selected to evaluate the PK profile of VCE-004.8 when orally administrated to rats. A formulation of 4 mg/mL of VCE-004.8 in Sesame Oil (Formulation n°6), and another with 4 mg/mL of VCE-004.8 with Sesame Oil (97.5%)-Ethanol (2.5%) (Formulation n°7) were prepared for the PK analysis in rats.

### Example 11

### Indena Phytosomes

Phytosome® is a patented technology developed by Indena Spa (Italy), a leading manufacturer of drugs and nutraceuticals. Phytosomes are little cell-like structures that contain the active ingredients bound to phospholipids, mainly phosphatidylcholine. The phospholipid molecular structure includes a water-soluble head and two fat-soluble tails. Because of this dual solubility, the phospholipids act as an effective emulsifier which produces a lipid compatible molecular complex. This phytosome technology is a breakthrough model for marked enhancement of bioavailability, significantly greater clinical benefit, assured delivery to the tissues, and without compromising nutrient safety.

A laboratory process for the preparation of VCE-004.8 phytosomes starting form VCE-004.8 was developed, first a solvent screening was performed, selecting ethyl acetate for comparison with ethanol, methanol acetone and dichloromethane and ethyl acetate. Two Phytosome-VCE-004.8 prototypes were prepared:

1) Phytosome 1:2 ratio, wherein Emulphur/SF (2 g), VCE-004.8 (1 g) and Maltodextrin MD05 (0.92 g) were suspended in 40 ml of ethyl acetate. The suspension was refluxed with stirring for 1 hr. The solvent was removed under reduced pressure (300-400 mbar, external bath at 60° C.) until a soft mass was obtained. The soft residue was dried under vacuum at 50° C. for 16 hr. To the dried solid 2% W/W of Syloid 244 FP was added. The solid was coarsely ground and sieved at 600 μm to yield VCE-004.8 phospholipid/SF. The weight yield vs. sum of starting powders was about 98% W/W.

2) Phytosome 1:1 ratio, wherein Emulphur/SF (1 g), VCE-004.8 (1 g) and Maltodextrin MD05 (1.92 g) were suspended in 40 ml of ethyl acetate. The suspension was refluxed with stirring for 1 hr. The solvent was removed under reduced pressure (300-400 mbar, external bath at 60° C.) until a soft mass was obtained. The soft residue was dried under vacuum at 50° C. for 16 hr. To the dried solid 2% W/W of Syloid 244 FP was added. The solid was coarsely ground and sieved at 600 μm to yield VCE-004.8 phospholipid/SF. The weight yield vs. sum of starting powders was about 98% W/W.

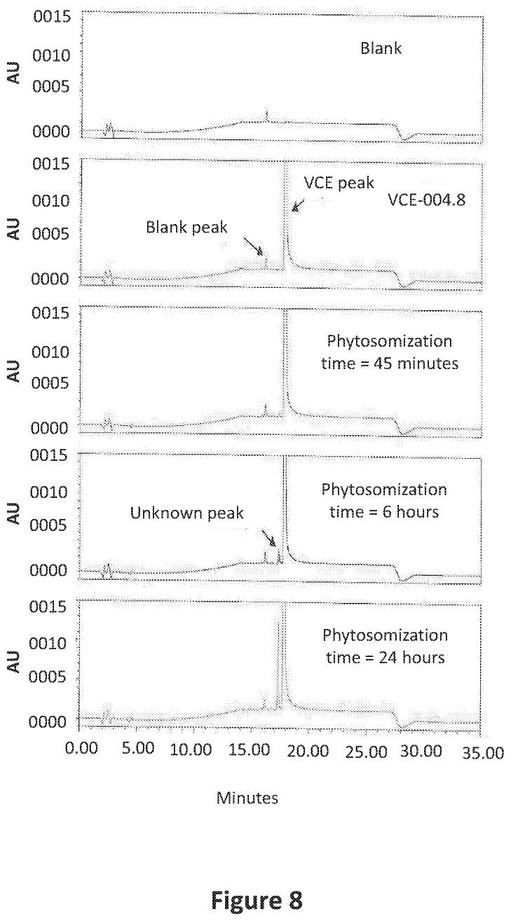

A preliminary investigation of the compound stability gave indication that the active principle is stable in the process conditions, although an impurity peak not detected in the starting material and almost neglectable at 45 mm, increased after 6 hr (1.7% in area %) and grew after 24 hr (6.2% in area %), as shown in FIG. 8.

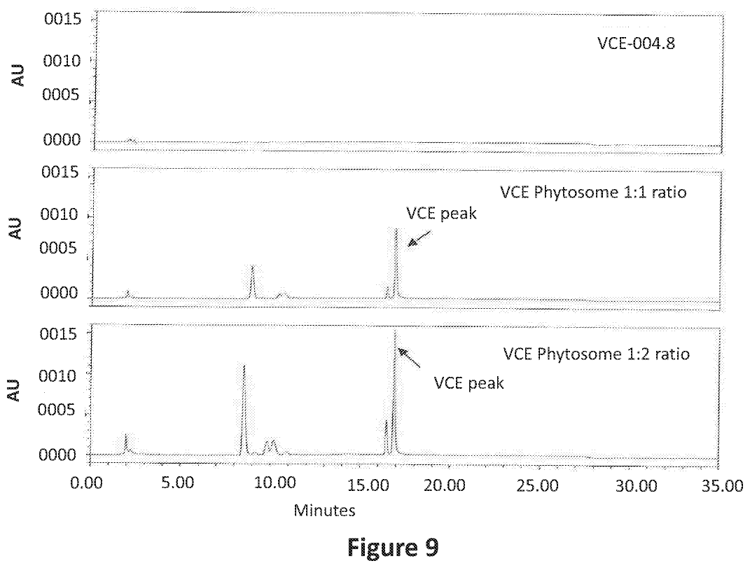

The solubility of Phytosome-VCE-004.8 was tested in buffer medium at various pH (1.2, 4, 5, 6, 8 and 8.0). For each pH, independent supersaturated solutions of VCE-004.8 and its phytosomes were prepared. The suspensions w ere sonicated for 10 min and kept in a water bath at 37° C. for 2 hr. Then the final suspensions were filtered (with 0.45 PTFE disposable fitter) and the solutions were injected for HPLC analysis. The results, shown in Table 12 and FIG. 9, indicated that the hydrophilicity (expressed as aqueous solubility) of VCE-004.8 is practically nil. However, the phytosomization process increase significantly the solubility of the compound. The 1:2 ratio resulted to be slightly more soluble than the 1:1 ration, and the best behavior was shown at neutral and basic pH. Accordingly. Phytosome 1:2 was selected for following assessment of PK profile in rats (Formulation n°8). Phytosome 1:2 contained 24% of VCE-004.8 and was prepared in Methyl cellulose 1% in water for oral administration.

TABLE 12
Concentration of VCE-004.8 found in the aqueous solutions of
VCE-004.8, Phytosome 1:1 and Phytosome 1:2 at the considered pH.

| | VCE-004.8 | Phytosome 1:1 | Phytosome 1:2 |
|---|---|---|---|
| | concentration | concentration | concentration |
| pH | (μg/ml) | (μg/ml) | (μg/ml) |
| 1.2 | 0.005 | 2 | 3 |
| 4.5 | 0.005 | 11 | 22 |
| 6.8 | 0.003 | 22 | 43 |
| 7.4 | 0.001 | 29 | 49 |
| 8.0 | 0.001 | 23 | 48 |

### Example 12

### Echo Pharmaceuticals ALITRA®

Alitra® is a drug delivery technology patented by Echo Pharmaceuticals BV. Alitra® is an emulsifying technology that was successfully developed and used by Echo to improve release of cannabinoids in aqueous solutions.

For the formulation of VCE-004.8, ECP012A was used, a mixture of excipients designed for oral use as base formulation. This renders a dry powder formulation of VCE-004.8 that was tableted to assess its consistency. For further investigational purposes, the three final VCE-004.8 formulations were delivered as powder.

VCE-004.8 ECP012A tablets were prepared through two manufacturing steps from the active ingredient VCE-004.8: a granulation step and a tablet preparation step. The first step was preparation of the intermediate product (IP): a granulating fluid containing excipients in ethanol was added to primary powder particles followed by solvent evaporation. The particle size of the resulting material was reduced by milling. This yielded the IF. a granulate ready for tableting. The second manufacturing step was preparation of the Drug Product (DP). The IP was blended with excipients and tablets were compressed by direct compression on a tablet press. Three different formulations were prepared as described in Table 13.

TABLE 13
Main features of three formulations VCE-004.8 using
Alitra ® technology.

| | Ratio | | | |
|---|---|---|---|---|
| | VCT-004.8 | VCE-004.8 | | Tablet |
| Formulation | to emulsifier | (% w/w) | Color | quality |
| A | 1:2 | 13.13 | Mat purple | Good solid |
| | | | with obvious | tablet |
| | | | white small | |
| | | | particles | |
| B | 1:1 | 13.75 | Purple with | Good solid |
| | | | white particles | tablet |
| C | 1:0.5 | 12.93 | Bright purple | Good tablet |
| | | | with few white | yet more |
| | | | particles | brittle than |
| | | | | A and B |

VCE004.8 content of the DP was measured by HPLC analysis in duplicate. Dionex Ultimate 3000 system operating under Chromclcon software. The HPLC method used is based on the United States Pharmacopoeia (USP) method for Dronabinol (delta-9-tetrahydrocannabinol, THC) and was developed for measuring of CBD and other cannabinoids.

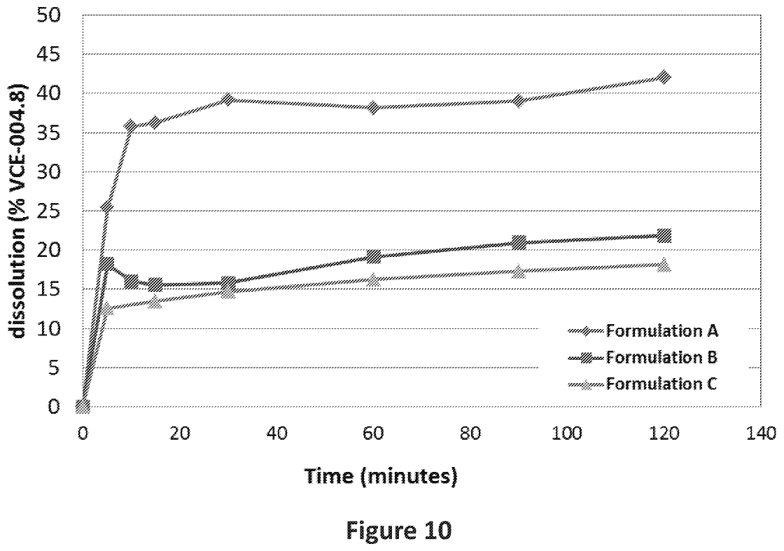

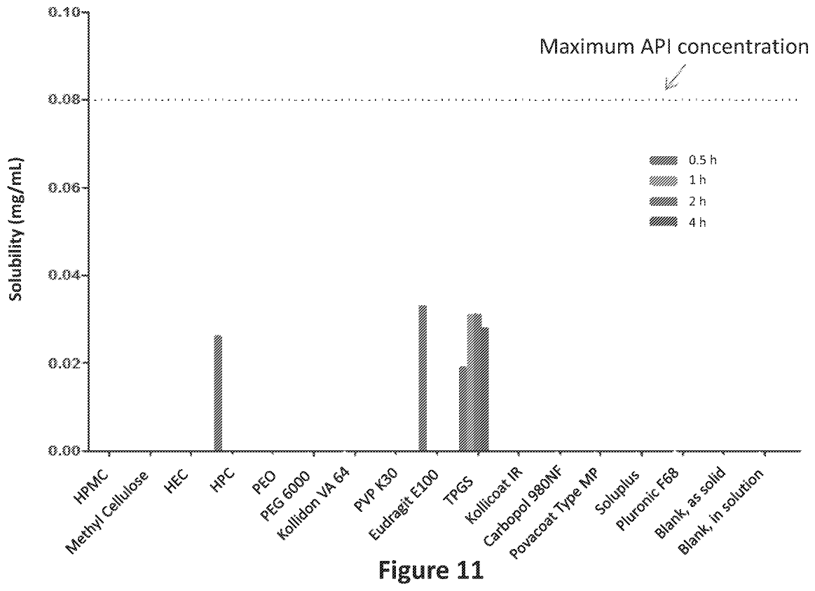

The dissolution test was used to indirectly determine the bioavailability of the API and to measure possible differences in bioavailability of the API in the different formulations. Dissolution was measured according to section 2.9.3 of the British Pharmacopoeia (BP). The selected dissolution medium consisted of 2% SDS in water, pH 7. A beaker was placed on a controlled heating mantle with stirring and a temperature between 35° C. and 40° C. Once the temperature of the dissolution medium reached 37° C. (t=0) the experiment was started by dropping one tablet into the dissolution beaker with a stainless-steel screen to create a physical barrier between the tablets and the stirrer bar. Samples were taken at various time points with a disposable syringe and were transferred to a vial for HPLC analysis. The dissolution is expressed as a percentage of the active substance that is dissolved in a specified time frame. Samples were taken at various time points: t=0, 5, 10, 15, 30, 60, 90 and 120 min. The results of the tests for the three formulations are shown in FIG. 10.

Results of the formulation test showed that Formulation A has the highest dissolution rate (reached 42%) followed by Formulation B and C. The order of dissolution rates is in line with expected effects of the API ratio to emulsifier: higher emulsifier to API ratio, better solubility. Although Formulation A showed better dissolution rate, the three formulations A, B and C were selected for assessing the PK profile in rats (Formulations n°9, 10, 11 respectively).

For the preparation of those formulations, it was taken into account that Formulation n°9 contained 13.13% of VCE-004.8, Formulation n°10 contained 13.75% of VCE-004.8 and Formulation n°11 contained 12.93% of VCE-004.8. It was prepared a 15 mg/ml suspension in water.

### Example 13

### Nanosuspensions

Ten different prototypes aqueous nanosuspension concepts were prepared as follows: 250 mg VCE-004.8 was weighed into a suitable container, to which 4.750 g of stabilizer solution was added. Each concept was stirred using a magnetic stirring bar until a homogenous suspension was formed. Next, to each container, 30 g beads (ZYP size 1 mm) were added, after which the container was sealed and placed on a roller mill at 80 rpm. After 2 days and 5 days, tire particle size distribution (PSD) of each concept was measured by laser diffraction. After 5 days of milling, all concepts were harvested and diluted to 10 mg/g, ensuring sufficient rinsing of the milling containers and beads. All ten concepts were placed on 25° C./60% RH stability conditions for 2 weeks, after which PSD was again evaluated. Results are shown in Table 14. From these results it is concluded that concept 2, containing 1% Pharmacoat 603+0.1% SLS as stabilizer, and concept 4, with 1% HPC-SSL+0.1% SLS, am to be considered for PK testing, since for these formulation concepts, the obtained d10-d50-d90 particle size results are all <1 μm.

TABLE 14
Different nanosuspension concepts with PSD results.

| | | Particle size distribution (μm) | | | | | | | | |
|---|---|---|---|---|---|---|---|---|---|---|
| | | After 2 days | | | After 5 days | | | After 14 days at | | |
| | | milling | | | milling | | | 25° C./60% RH | | |
| No° | Stabilizer (% w/w in H₂O) | d10 | d50 | d90 | d10 | d50 | d90 | d10 | d50 | d90 |
| 1 | 1% Pharmacoat 603 | 0.15 | 2.54 | 8.20 | 0.19 | 3.7 | 10.1 | 0.1 | 3.2 | 9.28 |
| 2 | 1% Pharmacoat 603 + 0.1% | 0.08 | 0.13 | 2.10 | 0.06 | 0.1 | 0.71 | 0.0 | 0.1 | 0.70 |
| 3 | 1% Nisso HPC-SSL | 0.12 | 1.63 | 6.15 | 0.09 | 0.1 | 4.17 | 0.0 | 0.1 | 4.13 |
| 4 | 1% Nisso HPC-SSL + 0.1% | 0.07 | 0.13 | 1.50 | 0.06 | 0.1 | 0.22 | 0.0 | 0.1 | 0.22 |
| 5 | 1% Kolliphor P188 | 0.23 | 7.19 | 19.4 | 3.10 | 9.5 | 21.4 | 0.2 | 7.4 | 22.06 |
| 6 | 1% Kolliphor P188 + 0.1% | 0.12 | 1.86 | 10.1 | 0.17 | 8.0 | 26.9 | 0.2 | 7.9 | 26.01 |
| 7 | 1% PVP K30 | 0.19 | 3.66 | 23.2 | 0.20 | 5.2 | 37.3 | 0.2 | 4.8 | 31.91 |
| 8 | 1% PVP K30 + 0.1% SLS | 0.12 | 1.56 | 7.59 | 0.10 | 1.3 | 7.89 | 0.1 | 1.4 | 8.67 |
| 9 | 1% PVP VA64 | 0.13 | 2.03 | 6.43 | 0.11 | 1.9 | 7.18 | 0.1 | 1.9 | 6.88 |
| 10 | 1% PVP VA64 + 0.1% SLS | 0.09 | 0.40 | 5.02 | 0.08 | 0.1 | 2.34 | 0.0 | 0.1 | 2.44 |

Nanosuspensions concept 2 and concept 4 were selected for PK supplies (Formulations n°12 and 13 respectively) and therefore freshly prepared as follows: 500 mg VCE-004.8 was weighed into a suitable container, to which 9.5 g of respective stabilizer was added. Each concept was stirred using a magnetic stirring bar until a homogenous suspension was formed. Next, to each container, 30 g beads (ZYP size 1 mm) were added, after which the container was sealed and placed on a roller mill at 80 rpm. After 24 hr and 45 hr, the particle size distribution (PSD) of each concept was measured by laser diffraction. After 45 hr of milling, all concepts were harvested and diluted to 10 mg/g. ensuring sufficient rinsing of the milling containers. Dose was adjusted from 10 mg/g to 4 mg/g. Therefore, three vials of each concept were pooled by magnetic stirring, after which each formulation was diluted 2.5 times with the respective stabilizer.

### Example 14

### Solid Dispersions

The development of solid dispersion formation started with the selection of polymers for stabilization of amorphous API. Therefore, multiple polymers were screened using the solvent shift method (Table 15).

TABLE 15
List of polymers used for the solvent shift in SIF and SGF based on
the solubility of polymers in these solutions. “S” marks polymers that
were dissolved and therefore, the solvent shift experiments were
performed; and “X” marks polymers that were not soluble and thus
the solvent shift experiments could not be performed.

| Polymer | SIF | SGF |
|---|---|---|
| HPMC-AS-MG | S | X |
| HPMC-AS-LG | S | X |
| HPMC-AS-HG | S | X |
| HPMC | S | S |
| HPMC-P-55S | S | X |
| HPMC-P-50, | S | X |
| Methyl Cellulose | S | S |
| HEC | S | S |
| HPC | S | S |
| Eudragit L100 | S | X |
| Eudragit E100 | X | S |
| PEO 100K | S | S |
| PEG 6000 | S | S |
| PVP VA64 | S | S |
| PVP K30 | S | S |
| TPGS | S | S |
| Kollicoat IR | S | S |
| Carbopol 980NF | S | S |
| Provocoat MP | S | S |
| Soluplus | S | S |
| Sureteric | X | S |
| Pluronic F-68 | S | S |

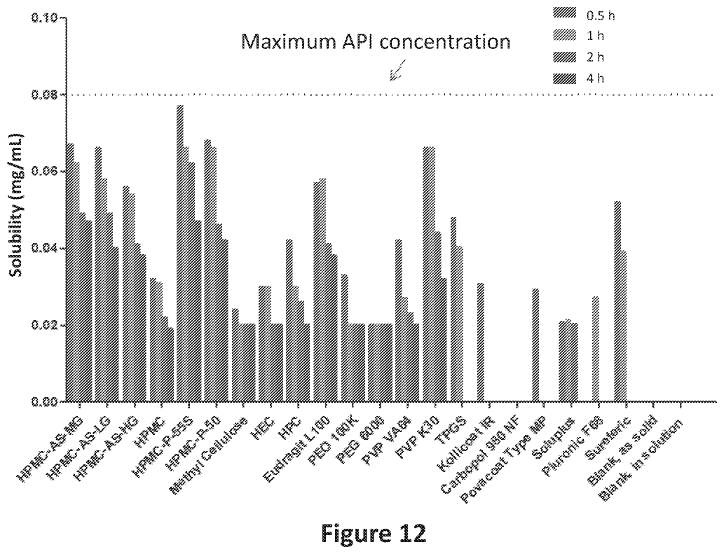

These experiments applied to a 5 mg/mL solution of VCE-004.8 in DMSO, of which 80 μL was added to 4 mL polymer solutions prepared in simulated intestinal fluid (SIF) and simulated gastric fluid (SGF). Subsequently the samples were incubated at 25° C. under continuous stirring, and after 0.5, 1, 2 and 4 hr, an aliquot was taken, filtered and analyzed by HPLC to determine the VCE-004.8 concentration in solution. Results are presented in FIG. 11 (SGF) and FIG. 12 (SIF).

In SGF, most polymers were not able to maintain a sustained supersaturated state, except for the TGPS solution, in which after 4 hr a concentration of about 0.03 mg-mL VCE-004.8 could be measured. However, the experiments performed in SIF showed several polymers with promising anti-precipitant properties. In general, all the HPMC derivates (except for HPMC as is) exhibit high API concentrations (approximately 60 μg/mL) from 0.5 to 1 hr. Moreover, Eudragit L100 and PVP K30 also maintain supersaturation for at least 1 hr (approximately 60 μg/mL). Therefore, these polymers are to be considered in the preparation of amorphous solid dispersions.

The principle behind a successful amorphous dispersion is to prepare a homogenous dispersion of the API in a polymer matrix, such that the mobility of the API molecules is reduced and nucleation is prevented. Drug loading is an important parameter and high drug loads may result in crystallization of the API, whereas low drug loads could affect the drug product size.

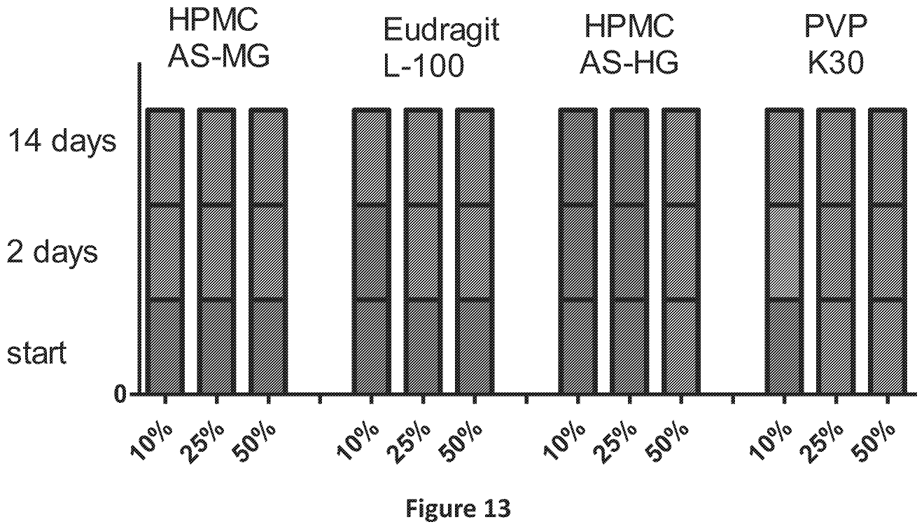

The amorphous solid dispersion screening (ASD) is performed with different drug loads of 10, 25 and 50%. Based on the polymer-API interaction observed by the solvent shift method, HPMC-AS-MG, Eudragit L100, HPMC-AS-HG and PVP K30 were selected for further investigation. The homogenous dispersions are prepared by freeze-drying and placed on 40°C./70% RH stability conditions. At preparation (T0) and after 2 days (T2D) and 14 days (T14D), samples arc analyzed by HT-XRPD. Results are shown in FIG. 13.

It is concluded that the HPMC-AS-HG dispersion is able to stabilize 10% and 25% drug load for at least 14 days at 40° C./75% RH. Eudragit L-100 can only stabilize 10% drug load for 2 days. HPMC-AS-MG and PVP K30 do not show stabilization during stability study.

Based on both the solvent-shift results and die amorphous solid dispersion stability screening, the two best performing polymers are MPMC AS HG and Eudragit L100 (Formulation n°14). However, as little (or no) release in the stomach is desired, but rapid release in the proximal small intestine is targeted, it was chosen to use HPMC AS LG (Formulation n°15) instead of HPMC AS HG, as the latter only dissolves at a rather high pH value of 6.8, while the LG grade already dissolves at pH 5.5.

The two solid dispersions selected were prepared by spray drying on ProScpT 4M8-TriX equipment. Prior to manufacturing, the optimal spray drying conditions were first determined by spray drying of placebo material (i.e., without VCE-004.8). The final settings used for each polymer are summarized in Table 16. After finalization of the spray drying process, the solid dispersion material was dried in a vacuum oven at 25° C. and 20 mbar for 16 hr. As dispersion medium, 0.5% Methocel E4M+0.2% Tween 20, was prepared.

TABLE 16
Spray drying conditions for the ProCepT 4M8-Trix spray dryer module

| Parameters ⁽¹⁾ | Eudragit L100 | HPMC AS LG |
|---|---|---|
| Solid mixture | VCE-004.8: | VCE-004.8: |
| | Eudragit L100, | HPMC AS LG, |
| Solvent mixture | Acetone: water, | Dichloromethane: |
| | 90:10, v/v | ethanol, |
| Air flow (m³/min) | 0.37-0.42 | 0.37-0.42 |
| Air inlet temperature | 97.8-100.1 | 98.4-98.9 |
| Product temperature | 46.5-49.1 | 47.6-48.7 |
| Pump speed (%) | 100 | 100 |
| Atomization pressure | 6.1-6.6 | 7.0 |
| Spray rate (g/min) | 5.6-6.1 | 7.0-7.2 |
| Yield (%) | 3 to 4 | 3 to 4 |

(⁽¹⁾ As this is a dynamic process, which is constantly being monitored, a dynamic range is given).

### Example 15

### Bioavailability Assessment in Rats

The PK study was performed in male Sprague Dawley rats and male Balb/C (C57BL/6JRj) mice around 6 weeks old supplied by Janvier Labs. There was entirely artificial lighting in the room with a controlled cycle of 12 h light, 12 h dark. It was air conditioned by a system designed to maintain normal conditions. Each animal was identified by an ear tag. Animals were examined for general health and welfare before the in vivo test. All animals had free access to food and water during the experiment (ad libitum). Standard process, treatment and euthanasia was conducted Several timepoints per formulation were selected (typically 5 min, 20 min, 30 min, 1 h, 3 h, 4 h, 8 h, 24 h for iv; and 30 min, 1 h, 2 h, 4 h, 6 h, 8 h, 10 h, 18 h, 24 h for oral administration). Usually, at least 3 animals per timepoint were used.

The test formulations were stored at 420 C. in the dark until the in vivo test was performed (usually in the following 4-6 days after the manufacturing). Formulation containing Maisine 35-1 was warmed to 37° C.-40° C. in a water bath and stirred (magnetic stirring), protected from light, before administration. Formulations were orally administrated to animals and compared with intravenous administration of VCE-004.8 dissolved 2 mg/mL in DMSO and administrated at a dose of 2-10 mg/Kg in a volume of 1 mL/kg. In mice, selected dose for oral administration was 20 mg/Kg in a 5 g/kg volume of administration. In rats, selected dose was 20-50 mg/kg.

For the blood sampling, at prescribed times, blood was collected in the sinus retro-orbital using a capillary tube. Approximately 0.5 ml. per time-point were collected. It was used lithium heparin as anticoagulant. Exact sampling times were noted tor each blood sampling. Blood samples were centrifuged at 2500 rpm at around 10° C., the plasma then removed and placed into labelled polypropylene tubes. Individual plasma samples were stored frozen (−20° C.±5° C.) until analysis.

The analysis of plasma samples, 100 μL of the plasma sample were taken and 300 μL of acetonitrile were added. After protein precipitation, analysis was performed using LC-MS/MS. For the analytical phase, the substance VCE-004.8 was dissolved at 1 mg/mL with appropriate solvent DMSO. For the Analytical test, the molecular and daughter ions were selected for the molecule after direct infusion info the MS-MS system. The analytical method consisted of a precipitation of the proteins by addition of acetonitrile followed by a LC-MS/MS analysis with C18 column. According to the expected sensitivity, at least 8 calibration standards were used for the preparation of the calibration curve in plasma. The corresponding correlation coefficient was calculated and had to be higher than 0.75 to continue with the in vivo test. The calibration range to be tested was 1 to 2000 ng/mL of plasma.

Estimation of PK parameters was performed using Kinetiea® (Version 4.3—Thermo Electron Corporation—Philadelphia—USA). The following parameters were estimated: maximal plasma concentration (Cmax (ng/mL)), first time to reach Cmax (Tmax (h)), area under the plasma concentration-time curve from administration up to the last quantifiable concentration at time t (AUCVt (ng/mL*h)), and absolute bioavailability

$$( F ( \% ) = \frac{AUC PO / dose PO}{AUC IV / dose IV} * 100 ) .$$

In rats, PK parameters obtained with the selected formulations and shown in Table 17 showed that the formulation of VCE-004.8 with Corn oil and Maisine 35-1 (0.4:49.8:49.8) led to the best bioavailability results. This bioavailability was confirmed in mice as shown in Table 18. A similar formulation (with Maisine CC instead of Maisine 35-1) was selected tor Phase I clinical studies, and named EHP-101 Liquid formulation.

TABLE 17
Pharmacokinetic parameters of several formulations of VCE-004.8 orally
administered and compared to intravenous administration in rats

| | | Cmax | Cmax | Tmax | AUCt | Dose | |
|---|---|---|---|---|---|---|---|
| n° | Formulation details | (ng/mL) | SD | (h) | (ng/mL*h) | (mg/kg) | Bioavailability |
| 1 | Miglyol ® 812 | 279.46 | 142.20 | 8.0 | 3,287.75 | 50.0 | 3.56% |
| | (Kollisolv ® | | | | | | |
| | MCT 70) | | | | | | |
| 2 | VCE-004.8: Corn oil: | 441.03 | 197.01 | 8.0 | 5,399.74 | 20.8 | 19.97% |
| | Maisine 35-1 | | | | | | |
| | (0.4:49.8:49.8) | | | | | | |
| 3 | VCE-004.8: Corn oil: | 198.30 | 73.78 | 2.0 | 2,023.42 | 20.0 | 7.73% |
| | Maisine 35-1; Kolliphor | | | | | | |
| | EL (0.4:32.4:32.4:34.8) | | | | | | |
| 4 | VCE-004.8: Captex: | 272.83 | 64.96 | 4.0 | 2,495.93 | 20.6 | 9.30% |
| | Capmul (0.4:49.8:49.8) | | | | | | |
| 5 | VCE-004.8: Captex: | 122.87 | 28.22 | 2.0 | 1,326.61 | 20.5 | 4.95% |
| | Capmul: Kolliphor EL | | | | | | |
| | (0.4:32.4:32.4:34.8) | | | | | | |
| 6 | Sesame Oil | 153.98 | 85.51 | 4.0 | 1,621.08 | 20.0 | 6.21% |
| 7 | Sesame Oil (97.5%)- | 160.60 | 126.45 | 4.0 | 1,030.87 | 20.0 | 4.85% |
| | Ethanol (2.5%) | | | | | | |
| 8 | Phytosome 1:2 (24% | 24.042 | 8.14 | 1.0 | 142.949 | 48.0 | 0.16% |
| | VCE-004.8) - Indena SpA | | | | | | |
| 9 | A- Alitra- Echo | 38.11 | 2.20 | 0.5 | 312.07 | 20.0 | 1.33% |
| | Pharmaceuticals BV | | | | | | |
| 10 | B- Alitra- Echo | 29.98 | 2.76 | 4.0 | 368.87 | 20.0 | 1.57% |
| | Pharmaceuticals BV | | | | | | |
| 11 | B- Alitra- Echo | 18.85 | 2.67 | 4.0 | 284.30 | 20.0 | 1.21% |
| | Pharmaceuticals BV | | | | | | |
| 12 | VCE-004.8: Pharmacoat | 67.44 | 39.54 | 0.5 | 557.05 | 19.9 | 2.15% |
| | 603: SLS: water | | | | | | |
| | (0.4:1:0.1:98.5) | | | | | | |
| 13 | VCE-004.8: HPC-SSL: | 87.08 | 9.69 | 0.5 | 574.05 | 20.3 | 2.17% |
| | SLS: water | | | | | | |
| | (0.4:1:0.1:98.5) | | | | | | |
| 14 | VCE-004.8: Eudragit L100 | 302.50 | 259.11 | 0.5 | 849.97 | 20.0 | 3.25% |
| | (10:90) | | | | | | |
| 15 | VCE-004.8: HPMC AS | 1,203.00 | 220.74 | 0.5 | 2,591.07 | 21.3 | 9.37% |
| | LG (10:90) | | | | | | |

(Note:
This table shows a selection of the results obtained for formulations 2 and 7. Complete results are shown in (8)).

TABLE 18
Pharmacokinetic parameters of formulation n° 15 orally
administered and compared to intravenous administration in mice.

| | | Cmax | Cmax | Tmax | AUCt | Dose | |
|---|---|---|---|---|---|---|---|
| n° | Formulation details | (ng/mL) | SD | (h) | (ng/mL*h) | (mg/kg) | Bioavailability |
| 2 | VCE-004.8: Corn oil: | 461.29 | 103.7 | 2.0 | 1,297.97 | 20.0 | 64.90 |
| | Maisine 35-1 | | | | | | |
| | (0.4:49.8:49.8) | | | | | | |

### Example 16

### Nonclinical Experience

On the basis of several in vitro biological assays, it was preclinically concluded that EHP-101 is: an activator of PPARγ signaling; a functional ligand agonist for the CB₂ receptor; and, a nonreactive aminoquinoid that modulates activation of the HIF pathway. Furthermore, a receptor screening study demonstrated VCE-004.8 specificity; there was no detectable affinity for the CB1 receptor, further supporting the lack of psychotropic effects. Thus, primary pharmacology studies were conducted to demonstrate the activity of EHP-101 in the treatment of MS using two standard multiple sclerosis (MS) murine models:

1) Experimental Autoimmune Encephalomyelitis (EAE) model that mimics human re lapsing-remitting MS (RRMS); and

2) Theiler Murine Encephalomyelitis Virus-induced demyelmating disease model (TMEV) that mimics progressive forms of MS. EHP-101 has demonstrated durable activity in these 2 models when it was administered both intraperitoneally and orally.

Primary pharmacology studies were also conducted to demonstrate the activity of EHP-101 in the treatment of systemic sclerosis (SSc) using a murine model of dermal fibrosis induced by bleomycin. SSc is a chronic multiorgan autoimmune disease of unknown etiology characterized by vascular and immunological abnormalities. Several lines of evidence have shown that the endocannabinoid system may play a role in the pathophysiology of SSc. Considering that the dual PPARγ/CB₂ agonists together with activation of the HIF pathway, have a strong potential as disease-modifying agents in SSc. EHP-101 was investigated for its activity in those targets.

For assessing Drug Metabolism and Pharmacokinetics (DMPK) and safety of EHP-101 Liquid, studies have been performed according to the International Council on Harmonisation (ICH) M3 guideline, encompassing in vitro and in vivo safety pharmacology studies (cardiovascular, respiratory, and CNS), in vitro metabolism, plasma protein binding, in vitro and in vivo genotoxicity studies, and general repealed-dose toxicity studies in rodent and nonrodent species up to a 28-day duration.

The EAE model demonstrated the preclinical efficacy of VCE-004.8 showing a highly significant therapeutic effect at doses of 5 mg/kg, 10 mg/kg, and 20 mg/kg, VCF-004.8 also significantly reduced microglial reactivity and infiltration of inflammatory cells while preserving myelin structure in the EAE animals. VCE-004.8 attenuated the clinical severity and neuropathology in TMEV model of MS, as measured by the actimeter test. The treatment with VCE-004.8 ameliorated the motor deficits in mice infected with Theiler's virus. VCE-004.8 significantly reduced microglial reactivity and infiltration of inflammatory cells and preserves myelin structure in TMEV-infected mice. VCE-004.8 treatment also reduced the number of infiltrated CD4⁺ T cells and immune cells in the spinal cord of TMEV mice. An intense demyelination, which was found in the spinal cord of TMEV mice, was significantly reduced by the treatment with VCE-004.8. It was found that axonal disorganization in TMEV mice was prevented by the treatment with VCE-004.8.

Studies were also conducted to show that the activity of EHP-101 is consistent with a dual PPARγ/CB₂ ligand agonist that prevents microglia activation, axonal degeneration, and demyelination in vivo. Additionally, in vitro studies performed with EHP-101 demonstrated that the molecule stabilizes the expression of HIF-1α and HIF-2α proteins in microglia, oligodendrocytes, and endothelial microvascular cell lines. HIF-1α stabilization induced the release of erythropoietin (EPO) and vascular endothelial growth factor (VEGF) A, which are known to be neuroprotective and have the potential for remyelination.

EHP-101 capacity to prevent fibrosis related to SSc and recover the vascular morphology was evaluated in the experimental model of SSc. VCE-004.8, the active principle substance of EHP-101 inhibited TGFβ-induced Col1A2 gene transcription and collagen synthesis in vitro. Moreover* VCE-004.8 inhibited TGFβ-mediated myofibroblast differentiation and impaired wound-healing activity. EHP-101 reduced dermal thickness, blood vessels collagen accumulation and prevented mast cell degranulation and macrophage infiltration in the skin. EHP-101 also prevented the reduced expression of vascular CD31 typical of skin fibrosis. In addition, RNAseq analysis of skin biopsies showed a clear effect of EHP-101 in the inflammatory and epithelial-mesenchymal transition transcriptomic signatures, qualifying EHP-101 as a candidate for the management of SSc.

### Psychotropic Effects and Abuse Potential

EHP-101 (i.e., VCE-004.8) does not bind and activate the CB1 receptor and therefore does not induce psychotropic effects, including sedation and catalepsy. There are no specific abuse-related studies at this time. Abuse-related AEs are AEs of special interest (AESIs) for this study and will be monitored for occurrence throughout the study (Section 10.4.1.1).

Several studies were performed in which it was shown that VCE-004.8 did not have an affinity for the cannabinoid CB1 receptor. It was shown in a screening study that the compound did not show affinity for the CB1 receptor at a concentration of 10 μM (4336 ng/mL). Considering the high plasma protein binding of VCE-004.8 (>99%) and conservative free fraction estimate of 1% in plasma, VCE-004.8 is highly unlikely to yield any clinically relevant CB₁ receptor affinity in vivo at total (unbound+bound) plasma concentration of at least up to 1 mM (433600 ng/mL). This plasma concentration is approximately 50-fold higher than the Cₘₐₓ values observed at no observed adverse effect level (NOAEL) in rats and in dogs after 4 weeks of treatment. Therefore, no clinically relevant effect on the CB1 receptor is anticipated in the clinical situation. Moreover, the only intermediate in the synthesis is VCE-004 (also called HU331), which has not been reported to bind to CB1 or to induce psychoactive effects in mice.

### Example 17

### EHP-101 Therapeutics

The therapeutic potential of EHP-101 in experimental models of MS. EHP-101 was shown to reduce neuroinflammation by acting on PPARγ/CB₂ receptors white also providing neuroprotection and potentially inducing re-myelination through the HIF pathway. EHP-101 treatment reduced both incidence and severity of clinical manifestations of the disease in experimental models of MS. Taken together these data indicate that EHP-101 may provide clinical benefit to MS patients by potentially being disease-modifying.

In addition, the therapeutic potential of EHP-101 (VCE-004.8) in SSc was also shown, providing evidence of the efficacy to alleviate skin inflammation, vascular damage and dermal fibrosis in the bleomycin murine model.

### Example 18

### Effects of EHP-101 on Inflammation and Remyelination in Murine Models of MS

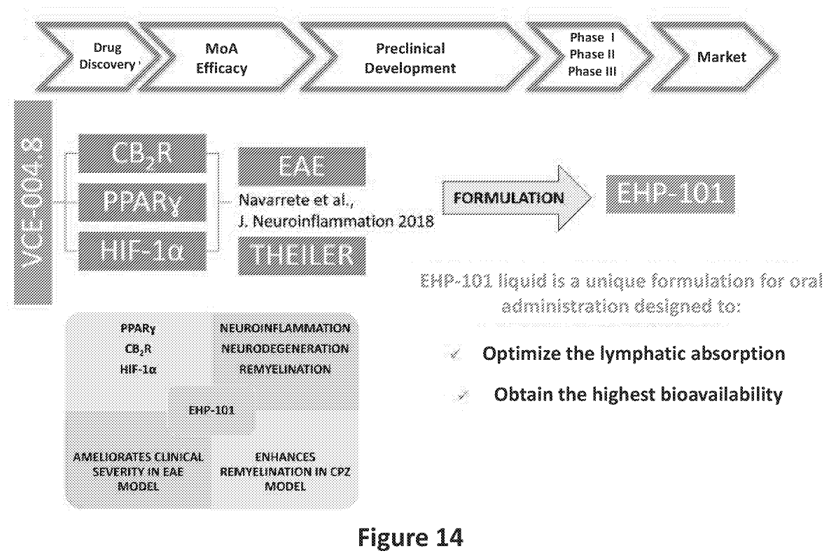

MS is characterized by a combination of inflammatory and neurodegenerative processes that are dominant in different stages of the disease. Thus, immunosuppression is the gold standard for addressing the inflammatory stage and novel remyclination therapies are being pursued to restore lost function. VCE-004-8 is a multitargeted synthetic cannabinoid derivative acting as a dual PPARγ/CB₂ ligand agonist that also activates the HIF pathway. VCE-004.8 was shown to prevent neuroinflammation, in two different models of MS (EAE and Theilers murine encephalitis virus-induced demyelinating disease). Oral EHP-101 (a lipidic formulation of VCE-004.8) showed a dose-dependent efficacy profile with prevention of neuroinflammation in the EAE model (FIG. 14).

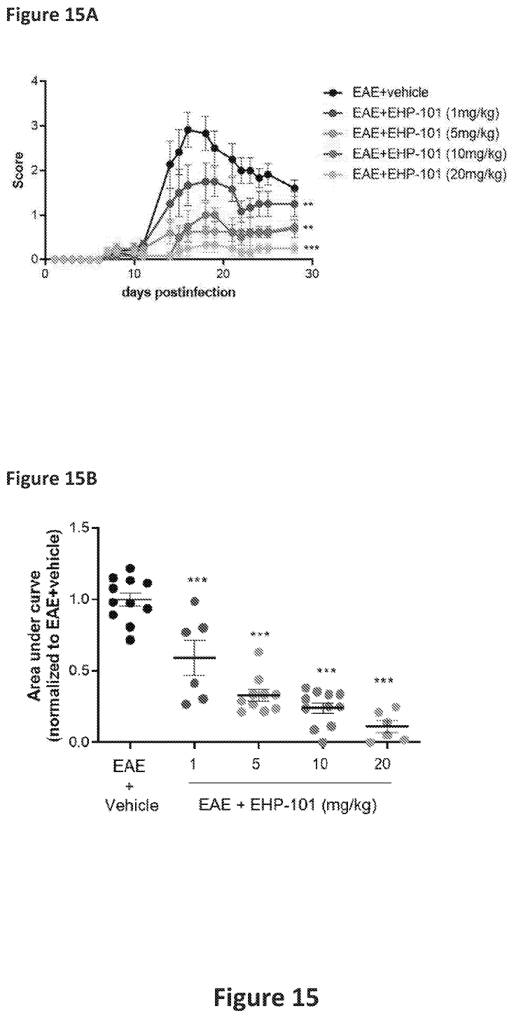

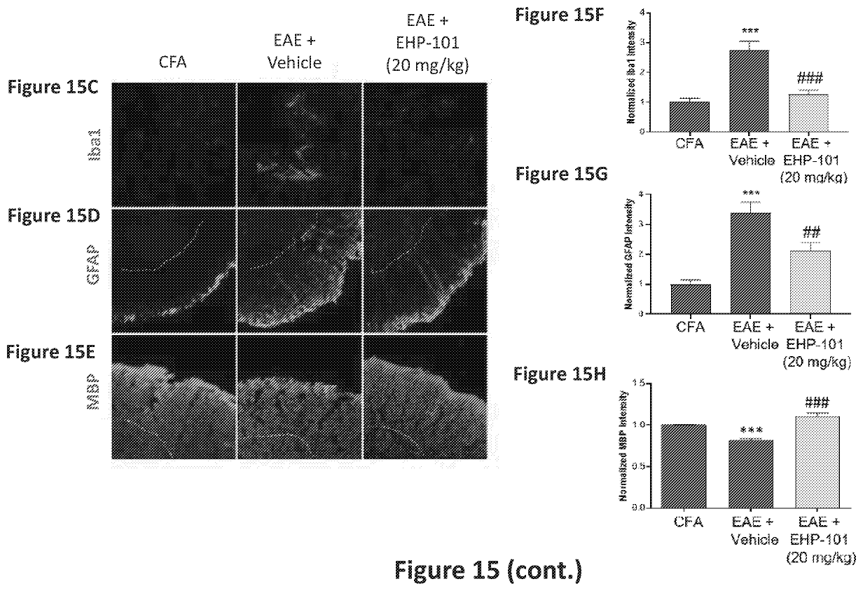

In EAE, transcriptomic analysis by RNA-Seq and qPCR demonstrated that EHP-101 prevented the expression of a large number of genes closely associated with MS pathophysiology in the spinal cord. In addition, EHP-101 normalized the expression of several genes associated with oligodendrocyte function, such as Teneurin 4 (Tenm4) that was downregulated in EAE. Immunohistochemistry analysis confirmed the recovery of Tenm4 expression in the spinal cord. Confocal analysis revealed that EHP-101 treatment prevented microglia activation (Iba1 staining), and demyelination (MBP staining) in both the spinal cord and the brain. Moreover, EAE was associated with a loss in the expression of Olig2 in the corpus callosum, a marker for oligodendrocyte differentiation, which was restored by EHP-101 treatment. In addition. EHP-101 enhanced the expression of glutathione S-transferase pi (GSTpi), a cytosolic isoenzyme used as a marker for mature oligodendrocytes in the brain. These data are indicative of the potential of EHP-101 to prevent demyelination in an MS murine model FIG. 15 through FIG. 1K).

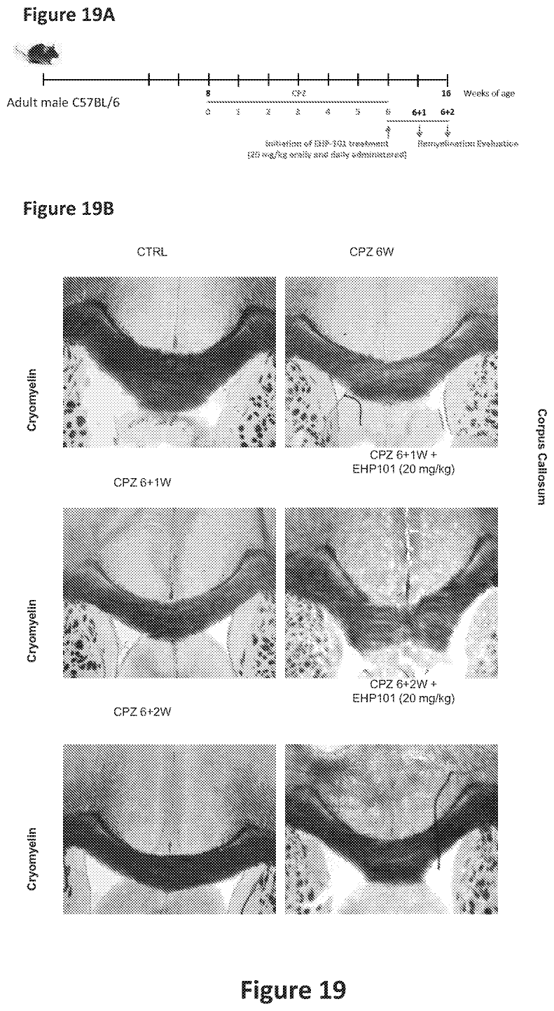

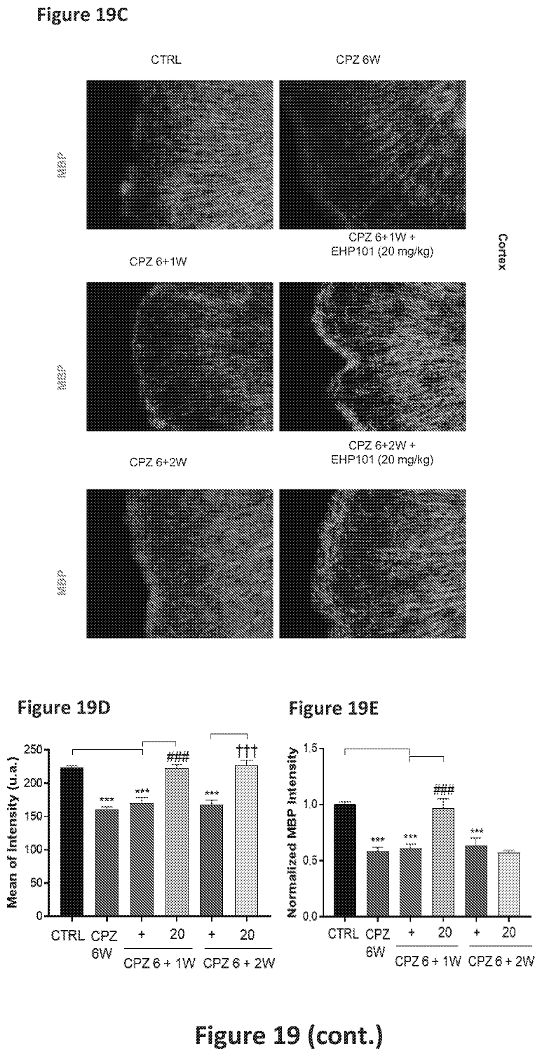

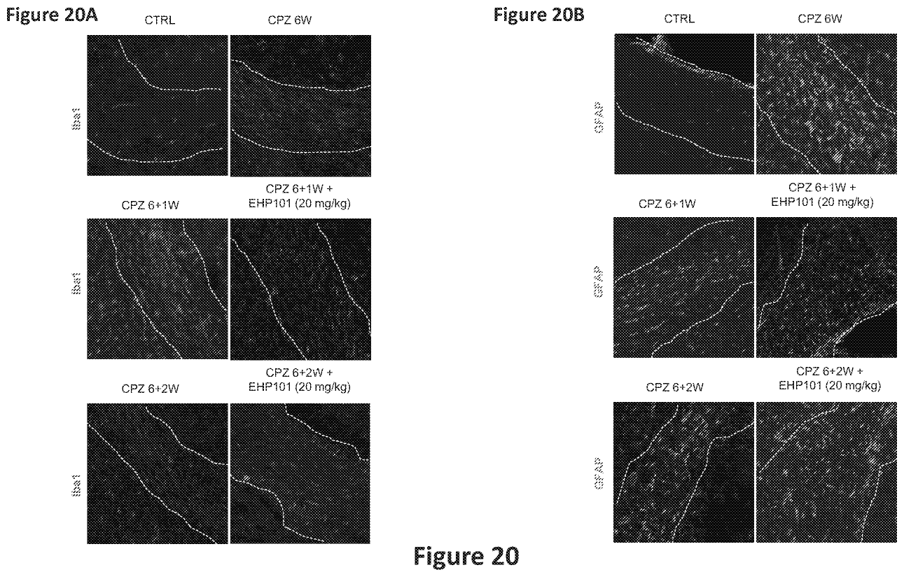

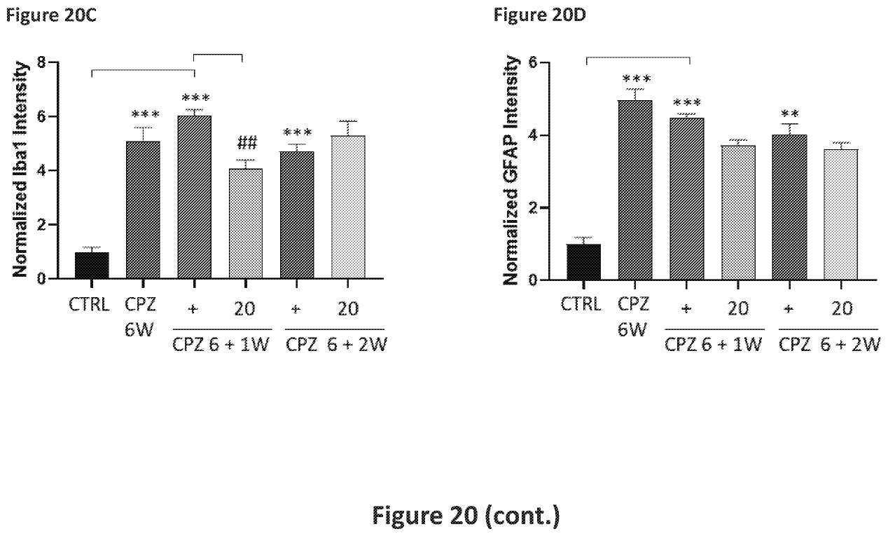

To further evaluate the potential of EHP-101, the effect of EHP-101 in a cuprizone model of demyelination was investigated. Mice were fed with a diet containing 0.2% cuprizone for 6 weeks and then the animals were switched to a normal diet and either treated or not treated (control) with EHP-101 (10 and 20 mg/kg) for 2 weeks. Cuprizone induced a clear loss of myelin in the brain measured by eryomyelin staining and MPB expression. Spontaneous recovery from demyelination was negligible after 1 and 2 weeks but remyelination was significantly accelerated by EHP-101 treatment. Moreover, EHP-101 also prevented cuprizone-induced microglial activation and asirogliosis detected by Iba1 and GFAP staining, respectively (FIG. 19 through FIG. 20).

In conclusion, EHP-101 represents a possible drug candidate for treatment of various diseases and disorders, such as different forms of MS and other demyelinating diseases.

### Example 19

### EHP-101 and Remyelination

### Methods of Example 19

Compounds

EHP-101 is a lipid-based formulation of VCE-004.8 [(1′R,6′R)-3-(Benzylamine)-6-hydroxy-3′-methyl-4-pentyl-6′-(prop-1-en-2-yl) [1,1′bi(cyclohexane)]2′,3,6-triene-2,5-dione)]. The chromatographic purity of VCE-004.8 in EHP-101 was 97.6%.

Cuprizone-Induced Demyelination Model

To induce demyelination, 8-week old C57BL/6 male mice were fed with 0.2% cuprizone TD. 140800 diet (Envigo, Barcelona, Spain) for six weeks. Control group (no demyelination) was fed with control mouse TD.00217 diet (Envigo, Barcelona, Spain) for the entire period. To study the effect on remyelination. EHP-101 was administered daily by oral gavage at 20 mg/kg from week six. For comparison, animals in the cuprizone control group post-demyelination received the same volume of vehicle by oral gavage. To study the dynamic effect of EHP-101 on remyelination, animals in each group were sacrificed at weeks 6, 7 (6W), 8 (6+2 W) post-treatment for further analysis.

Tissue Processing

Mice were anesthetized by i.p. administration with a ketamine-xylazine solution and they were transcardially perfused with saline 0.9%. Brains were fixed, cryoprotected and frozen at −80° C. for further analysis.

Immunohistochemistry Analysis

For antigen retrieval brain sections were boiled tor 10 min in sodium citrate buffer (10 mM, pH 6.0) or Tris-EDTA buffer (10 mM Tris Base, 1 mM EDTA 0.05% Tween 20, pH 9.0) (Sigma-Aldrich, St. Louis, Mo., USA). The sections were washed three times in PBS. Nonspecific antibody-binding sites were blocked for 1 h at room temperature with 3% bovine serum albumin (BSA) (Sigma-Aldrich, St. Louis, Mo., USA in PBS). Next, the sections were incubated overnight at 4° C. in following primary antibodies diluted in PBS with 3% BSA: microglia cells were stained with a rabbit anti-Iba-1 antibody (1:1,000: Wako Chemical Pure Industry, Osaka, Japan), astrocytes were stained with a mouse anti-GFAP antibody (1:500, Santa Cruz Biotechnology, Santa Cruz, Calif., USA), myelin basic protein was marked with a rabbit anti-Myelin Basic Protein antibody (1:1000; Abeam, Cambridge, UK). After extensive washing in PBS, slides were incubated with secondary antibodies for I h at room temperature in the dark. The immunoreactions were revealed using anti-rabbit Texas Red (1:100), anti-mouse/rabbit Alexa 488 (1:100) obtained from Thermo Fischer Scientific, Walthamm, Mass., USA. The slides were then mounted using Vectashield Antifade Mounting Medium with DAPI (Vector Laboratories, Burlingame, Calif., USA). Myelin integrity was analysed using the Hito CryoMyelinStain™ Kit (Gold phosphate complex Myelin Staining Kit) following manufacturer's recommendation (Hitobiotech Corp., Kingsport, Tenn., USA). All images were acquired using a spectral confocal laser-scanning microscope LSM710, (Zeiss, Jena, Germany) with a 20×/0.8 Plan-Apochrormat lens and quantified in 9-15 randomly chosen fields using ImageJ software (rsbweb.nih.gov/ij/).

Data Analysis

All the in vivo data ate expressed as the mean±SUM. One-way ANOVA followed by the Tukey's post hoc test for parametric analysis or Kruskal-Wallis post hoc test in the case of non-parametric analysis tests were used to determine the statistical significance. The level of significance was set at p<0.05. Statistical analyses were performed using Graph Pad Prism version 8.00 (GraphPad, San Diego, Calif., USA).

### Results of Example 19

### EHP-101 Accelerates Remyelination in a Cuprizone-Challenged Mouse Model

To evaluate the effect of EHP-101 on myelin damage in a CPZ-induced demyelination model (FIG. 19A), brain coronal sections from animals after 6 weeks of CPZ 0.2% diet and 2 weeks of EHP-101 treatment were analyzed. In this model, EHP-101 treatment began after CPZ diet removal, to more directly evaluate formulation effects on remyelination. First, the evaluation of MBP (cortex) w as determined both immunohistochemistry and Cryomyelin (corpus callosum) (FIG. 19C and FIG. 19B, respectively) staining where myelin was stained using a gold phosphate complex myelin staining kit in stained preparations, and myelin is intensely black. Spontaneous recovery from demyelination was insignificant after 1 and 2 weeks but remyelination was significantly accelerated by EHP-101 treatment, interestingly, both studies showed EHP-101 to enhance remyelination in Corpus Callosum in the ease of staining (FIG. 19D p=<0.0001 CPZ6W, CPZ6+1W, CPZ6+2W vs Control; p=<0.0001 CPZ6+1W+EHP-101 20 mg/kg vs CPZ6+1W; p=<0.0001 CPZ64+2W+EHP-101 20 mg/kg vs CPZ6+2W) and Cortex throughout immunohistochemistry studies (FIG. 19E p=<0.0001 CPZ6W, CPZ6+1W. GPZ6+2W vs Control; p=<0.0001 CPZ6+1W+EHP-101 20 mg/kg vs CPZ6+1 W). Moreover, the effect of EHP-101 on neuroinflammation-associated glial activation was also investigated using immunofluorescence staining of Iba-1 and GFAP in the Corpus Callosum. In control mice microglia and astrocytes were detected at low levels. Mice exposed to CPZ showed microglial and astrocytic hypertrophy, which were attenuated by EHP-101 treatment ( FIG. 20A and FIG. 20B). Quantitative assessment also showed a significant increase in the number of Iba1+ and GFAP+cells in Corpus callosum upon CPZ intoxication. Microgliosis and astrocytic reactivation was ameliorated after 1 week of EHP-101 treatment (FIG. 20C p=<0.0001 CPZ6W, CPZ6+1W, CPZ6+2W vs Control; p=0.0017 CPZ6+1W+EHP-101 20 mg/kg vs CPZ6+1W; FIG. 20D p=<0.0001 CPZ6W, CPZ6+1W vs Control; p=0.0017 CPZ6+2W vs Control).

### Example 20

### Effects of EHP-101 on Inflammation and Remyelination in Murine Models of Multiple Sclerosis

MS is an autoimmune disease that affects the CNS and is characterized by pathological changes, including neuroinflammation, demyclination and axon injury. The spontaneous repair of damaged myelin sheaths and axons has been described during the remission period of classical relapsing-remitting MS (RRMS), where demyelinated axons could be rewrapped by the regenerated myelin sheath, thus ameliorating axonal dysfunction. In this sense, the remission period is also considered the period of remyelination, which is important because it could be a key time point for the treatment of RRMS patients with drugs preventing inflammation and enhancing remyelination.

Small molecules including cannabinoids acting at druggable targets of the endocannabinoid system (ECS) are being explored for the management of CNS pathologies including MS. In this sense, several lines of evidence suggested a role for the ECS in oligodendrocyte function and remyelination activity in MS. The ECS is composed by the G-protein couple receptors CB1 and CB2, endocannabinoids and the enzymes regulating their synthesis and catabolism. In addition, cannabinoids of different nature also target ionotropic receptors of the TRP family and nuclear receptors such as peroxisome proliferator-activated receptors (PPARs). CB1 receptors arc expressed mainly in the CNS at neuronal terminals and regulate neurotransmitter release and psychoactive processes. In contrast. CB2 receptors are located primarily on the peripheral immune system, and during neuroinflammation on activated microglia in the CNS. Key considerations for developing CB2 receptor agonists include absence of psychoactive effects, sustained anti-inflammatory activity, tissue/cell protection, lack of cardiovascular adverse effects and efficacy in several disease models on neuroinflammation including MS.

PPARs are members of the nuclear hormone receptor superfamily of ligand-activated transcriptional factors with well-identified regulatory roles in lipid and glucose homeostasis and adipocyte differentiation. In addition to adipocytes and hepatocytes, PPARγ has been shown to be expressed in different CNS cells and in immune cells. Furthermore, PPARγ has been described as an important factor in the regulation of the immune response. In this sense, PPARγ activation has been shown to suppress the expression of inflammatory cytokines in astrocytes and macrophages/microglia. Furthermore, PPARγ stimulated oligodendrocyte differentiation from neural stem cells, promoted and accelerated the differentiation of oligodendrocyte progenitor cells in vitro with an additional increase in antioxidant defences and increased lipid production and terminal differentiation of cultured oligodendrocytes, thus suggesting an additional possible protective role of PPARγ in MS as a mediator of remyclination. The neuroprotective effects of PPARs, including PPARγ, have also been widely documented in vitro in various experimental paradigms of neurodegeneration, broadening its potential therapeutic perspectives in MS.

Although most current therapies for MS are directed towards modulation of the exacerbated immune response, novel therapies aimed to axonal remyclination are urgently needed. A novel approximation to achieve this would be the hypoxia preconditioning process which, induced by mild oxygen depletion, is beneficial in a wide number of neurological disorders, including MS. The cellular adaptation to severe or mild hypoxia is very fast and involves the activation of the hypoxia-inducible factor-1α (HIF), whose activation may play a role in the inflammatory and the remitting phases of MS. In addition, there is evidence suggesting that activation of the HIF pathway may also be linked to neuroprotection and perhaps remyelination. For instance, erythropoietin (EPO), whose gene is dependent on HIF activation, is neuroprotective in different animal models of MS.

It was previously shown that VCE-004.8 is a promising cannabidiol derivative acting as a dual agonist of PPARγ and CB2 that also activate the HIF pathway. Indeed, VCE-004.8 prevented neuroinflammation and demyelination in two different murine models of MS, such as EAE and Theilet's virus-induced encephalopathy. EHP-101 is an oral formulation of VCE-004.8 that showed efficacy in a murine model of systemic sclerosis. More importantly, EHP-101 has completed a Phase I clinical study (clinicaltrial.gov: NCT03745001) and initiation of Phase II studies in SSc and MS patients are being planned. The present example shows the efficacy of EHP-101 in preventing neuroinflammation and demyelination in EAE and to enhance remyclination in the cuprizone model of demyelination.

### Methods of Example 20

Compounds

EHP-101 is a lipidic-based formulation of VCE-004.8 [(1′R.6′R)-3-(Benzylamine)-6-hydroxy-3-methyl-4-pentyl-6′-prop-1-en-2-yl) [1,1′bi(cyclohexane)]-2′,3,6-triene-2,5-dione)]. The chromatographic purity of VCE-004.8 in EHP-101 was 97.6%.

Animals

All experiments were performed in strict accordance with EU and governmental regulations. Handling of animals was performed in compliance with the guidelines of animal care set by the European Union guidelines 86-609/EEC, and the Ethics Committees on Animal Experimentation at the Cajal Institute (CSIC, Madrid) and the University of Cordoba (UCO, Córdoba, Spain) approved all the procedures described in this study (for EAE at Cajal Institute protocol number: 96 2013/03 CEEA-IC and for cuprizone model at UCO protocol number: 2018PI/02 (UCO). Measures to improve welfare assistance and clinical status as well as endpoint criteria were established to minimize suffering and ensure animal welfare. Briefly, wet food pellets are placed on the bed-cage when the animals begin to develop clinical signs to facilitate access to food and hydration. For EAE model female C57BL/6 mice were purchased from Harlan (Barcelona, Spain), in the case of cuprizone model male C56BL/6 mice were purchased from Janvier Labs (Le Genest-Saint-Isle, France). All animals were housed in the animal facilities under the following controlled conditions: 12 h light/dark cycle; temperature 20° C. (±2° C.) and 46-50% relative humidity with free access to standard food and water.

Induction and Assessment of EAE

EAE was induced in C57BL/6 female mice at 6-8 weeks of age by subcutaneous immunization with MOG35-55 (300 μg: peptide synthesis section, CBM, CSIC. Madrid, Spain) and 200 μg of Mycobacterium tuberculosis (H37Ra Difco, Franklin Lakes, N.J., USA) in a 1:1 mix with incomplete Freund's adjuvant (OFA, Sigma). On the same day and 2 days later, mice were injected intraperitoneally with 200 ng of pertussis toxin (Sigma) in 0.1 mL PBS. Control animals (CFA) were inoculated with the same emulsion without MOG and they did not receive pertussis toxin. Treatment started at day 8 post-immunization when animals showed the first symptoms of the disease and consisted in daily oral BHP-101 (1, 5, 10 and 20 mg/kg) for the following 21 days. The mice were examined daily for clinical signs of EAE and disease scores were measured as follows: 0, no disease; 1, limb tail; 2, limb tail and hind limb weakness; 3, hind limb paralysis; 4, hind limb and front limb paralysis; 5, moribund and death. All animals were sacrificed at 28 days for further analysis,

Cuprizone-Induced Demyelination

To induce demyelination 8-week old C57BL/6 male mice were fed with 0.2% cuprizone TD. 140800 diet (Envigo, Barcelona, Spain) for six weeks. Control group (no demyelination) was fed with control mouse TD.00217 diet (Envigo, Barcelona, Spain) for the entire period. To study the effect on remyelination, EHP-101 was administered daily by oral gavage at 20 mg/kg from week six. For comparison, animals in the cuprizone control group (maximal demyelination) received the same volume of vehicle by gavage. To study the dynamic effect of EHP-101 on remyelination. animals in each group were sacrificed at weeks 6,7 (6+1 W), 8 (6+2 W) for further analysis.

Tissue Processing

Mice were anesthetized by i.p. administration with a ketamine-xylazine solution and they were transcardially perfused with saline 0.9%. The spinal cord was obtained by extrusion with saline. Brain and cervical spinal cord were immediately frozen and kept at −80° C. for RT-PCR analysis, the remaining brain and spinal cord were fixed in 4% paraformaldehyde in 0.1 M PBS. washed in 0.1 M PBS, cryoprotected with a 15% and then a 30% solution of sucrose in 0.1 M PBS, and frozen at −80° C. Free-floating brain and thoracic spinal cord sections (50 μm thick; Leica Microsystems CM1900 cryostat, Barcelona, Spain) were then processed for immunohistochemistry or immunofluorescence. In the case of cuprizone model w hole brains were fixed, cryoprotected and frozen at −80° C. for further analysis.

Immunohistochemistry Analysis

For IHC analysis, free-floating thoracic spinal cord (50 μm) sections were washed with 0.1M PB. Endogenous peroxidase activity was inhibited with 3.3% hydrogen peroxide in methanol. The sections were blocked with 2.5% normal horse serum and then incubated overnight at 4° C. in blocking buffer with a rabbit anti-Teneurin 4 antibody (1:50: Novus Biological, Colo., USA). Slides were incubated with ImmPRESS reagent (Vector Laboratories; Burlingame, Calif., USA) and then developed with diaminobenzidine chromogen (Merck, Darmstadt, Germany). Samples were photographed, digitalized using a Leica DFC420c camera and analyzed using Image J software. Myelin integrity was analyzed using the Hito CryoMyelmStain™ Kit (Gold phosphate complex Myelin Staining Kit) following manufacturer's recommendation (Hitobiotech Corp., Kingsport, Tenn., USA).

Confocal Microscopy Analysis

For antigen retrieval, spinal cord or brain sections were boiled tor 10 min in sodium citrate buffer (10 mM, pH 6.0) or Tris-EDTA buffer (10 mM Tris Base, 1 mM EDTA 0.05% Tween 20, pH 9.0) (Sigma-Aldrich, St. Louis, Mo. USA). The sections were washed three times in PBS. Nonspecific antibody-binding sites were blocked for 1 h at room temperature with 3% bovine serum albumin (BSA) (Sigma-Aldrich, St. Louis, Mo., USA in PBS). Next, the sections were incubated overnight at 4° C. with the following primary antibodies diluted in PBS with 3% BSA: microglia cells were stained with a rabbit anti-Iba-1 antibody (1:1,000; Wako Chemical Pure Industry, Osaka, Japan), astrocytes were stained with a mouse anti-GFAP antibody (1:500, Santa Cruz Biotechnology, Santa Cruz, Calif., USA), myelin basic protein was marked with a rabbit anti-Myelin Basic Protein antibody (1:1000; Abeam, Cambridge, UK), oligodendrocytes were marked with a mouse anti-Olig2 (1:100, Santa Cruz, Calif., USA) and a rabbit anti-GSTPi (1:250, Abeam, Cambridge, UK) axonal damage was determined with a mouse anti-Neurofilament H (NF-H) Nonphosphorylated antibody (SMI-32) (1:50; Biolegend, Calif., USA). After extensive washing in PBS. slides were incubated with secondary antibodies for 1 h at room temperature in the dark. The immunoreactions were revealed using anti-rabbit Texas Red (1:100), anti-mouse/rabbit Alexa 488 (1:100) obtained from Thermo Fischer Scientific, Wakhamm, Mass., USA. The slides were then mounted using Vectashield Antifade Mounting Medium with DAPI (Vector Laboratories, Burlingame, Calif., USA). All images were acquired using a spectral confocal laser-scanning microscope LSM710, (Zeiss, Jena, Germany) with a 20×/0.8 Plan-Apochromat lens and quantified in 9-15 randomly chosen fields using Imaged software (rsbweb.nih.gov/ij/).

RNA-Seq and Bioinformatic Analysis

Total RNA was isolated front spinal cord tissue using QIAzol lysis reagent (Qiagen, Hilden, Germany) and purified with RNeasy Lipid Tissue Mini kit (Qiagen). Then, samples were processed for high throughput sequencing using poly-A selection with the TruSeq Stranded mRNA Library Prep Kit (Cat. No. RS-122-2101, Illumina, San Diego, Calif., USA). In brief, 1 μg of total RNA from each sample was used to construct a cDNA library, followed by sequencing on the Illimina HiSeq 2500 system with single end 50 bp reads and ˜40 millions of reads per sample (n=3 per group). FASTQ files were pre-processed with Trimmomatic (v0.36) and aligned to mouse genome assembly mm10 using HISAT2 (v2.1.0). Then, counts per gene matrix were obtained with featureCounts (v1.6.1) using the in-built RefSeq annotation for mm10 genome assembly and the differential expression analysis was earned out using DESeq2 (v1.20.0), excluding genes with less than 15 counts across all samples. The functional over-representation analyses were performed using EnrichR and clusterProfiler. All the P values were adjusted to control the false discovery rate (FDR) using the Benjamini and Hochberg approach. RNA-seq data have been deposited in the Gene Expression Omnibus databank (accession no. GSE131854).

Quantitative Reverse Transcriptase-PCR

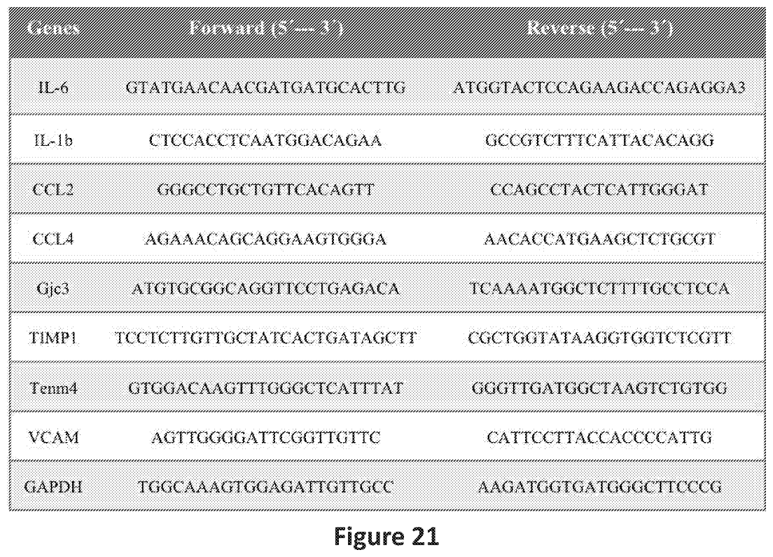

Total RNA (1 μg) was retrotranscribed using the iScript cDNA Synthesis Kit (Bio-Rad, Hercules, Calif., USA) and the cDNA analyzed by real-time PCR using the iQTM SYBR Green Supermix (Bio-Rad) and a CFX96 Real-time PCR Detection System (Bio-Rad). GAPDH gene was used to standardize mRNA expression in each sample. Gene expression was quantified using the 2-ΔΔCt method and the percentage of relative expression against controls was represented. The primers used in this study are described in FIG. 21.

Determination of Neurofilament, Light Polypeptide (NFFL)

Blood samples were taken under general anesthesia, and Lithium-Heparin plasma was collected. Samples were centrifuged for 20 min at 2000×g within 30 min of collection, and circulating levels of Neurofilament. Light Polypeptide (NHFL), were quantified with an Enzyme-linked Immunosorbent Assay Kit for Neurofilament Light Polypeptide (NHFL) (Cloud Clone Corp./USCN Life Science, Houston, Tex., USA) according to the manufacturer's instructions. Values were normalized versus control group and correspond to mean±SEM of 4 to 6 animals per group.

Data Analysis

All the in vivo data are expressed as the mean±SEM. One-way ANOVA followed by the Tukey's post hoc test for parametric analysis or Kruskai-Wallis post hoc test in the case of non-parametric analysis tests were used to determine the statistical significance. The level of significance was set at p<0.05. Statistical analyses were performed using GraphPad Prism version 8.00 (GraphPad, San Diego, Calif., USA).

### Results of Example 20

EHP-101 Attenuates Clinical Severity and Neuroinflammation in EAE

The efficacy of EHP-101 in MS was first evaluated in EAE, performing the treatments at an early stage of the disease since mice received increasing doses of EHP-101 at day 8 p.i. (post-immunization). Subcutaneous immunization with MOG35-55 induced EAE in all mice that received the vehicle alone. All vehicle-treated mice developed a disease that peaked by day 16 p.i. and maintained at day 28 p.i. By contrast, animal's score showed therapeutic efficacy of EHP-101 with all the doses tested, being the higher dose (20 mg/kg) able to prevent the symptoms completely (FIG. 15A p=0.0002 EAE+EHP-101 20 mg/kg vs EAE+Vehicle; p=0.0046 EAE+EHP-101 10 mg/kg vs EAE+Vehicle; p=0.0068 EAE+EHP-101 5 mg/kg vs EAE+Vehicle). Clinical score data from FIG. 15A were used to determine the area under curve and it is showed in FIG. 15B (p<0.0001 EAE+EHP-101 1/5/10/20 mg/kg vs EAE+Vehicle) that EHP-101 improved symptomatology in a dose-dependent manner.

To determine whether EHP-101 was able to target neuroinflammation in EAE, microgliosis and astrogliosis were evaluated in the spinal cord. Histopathological analysis showed that the extensive microglia/macrophage activation (FIG. 15C through FIG. 15F p=0.0003 EAE+Vehicle vs CFA; p=0.0006 EAE+EHP-101 20 mg kg vs EAE+Vehicle) and astrocyte activation (FIG. 15C through FIG. 15E, FIG. 15G p<0.0001 EAE+Vehicle vs CFA; p=0.0051 EAE+EHP-101 20 mg/kg vs EAE+Vehicle) in the spinal cord of EAE mice evidenced by both Iba-1 and GFAP staining was greatly reduced by EHP-101. MS pathology is characterized by focal demyelinating lesions in the CNS at both spinal cord and brain levels. Therefore, to determine the extent of demyelination, myelin was evaluated by MBP immunolabelling. A clear demyelination was found in the spinal cord of EAE mice that was significantly prevented by EHP-101 treatment (FIG. 15C through FIG. 15E, FIG. 15H p=0.0001 EAE+Vehicle vs CFA; p<0.0001 EAE+EHP-101 vs EAE+Vehicle).

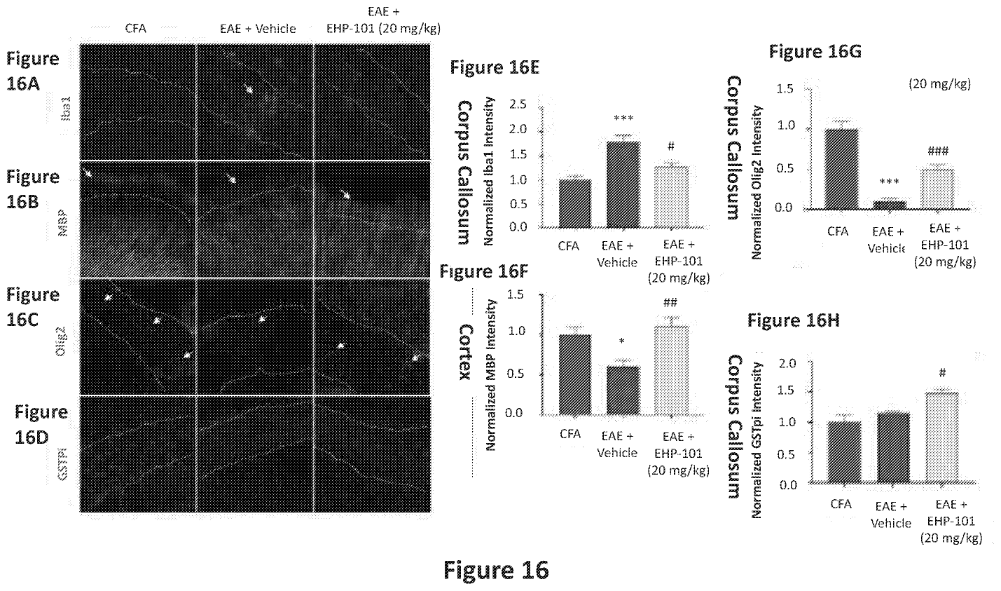

Cerebral conical demyelination as well as callosal pathology are widely recognized features of MS. In addition, the cerebral cortex plays a central role in interhemispheric communication, and callosal atrophy in MS patients has been shown to correlate with disability status. Therefore, it was also examined whether these structures might also be affected in EAE mice. An increase in inflammatory lesions was seen throughout the EAE forebrain (FIG. 16A through FIG. 16D). Specifically, it was observed that microglial reactivity was increased in Corpus callosum of EAE mice and tire treatment with EHP-101 reverted the microgliosis process (FIG. 16E p=0.0002 EAE+Vehicle vs CFA: p=0.0395 EAE+EHP-101 20 mg/kg vs EAE+Vehicle). Furthermore, brain sections from EAE-affected mice were also analyzed for the distribution of MRP reactivity. MBP immunoreactivity appeared significantly reduced in cerebral cortex (FIG. 16F p=0.0159 EAE+Vehicle vs CFA; p=0.0024 EAE+EHP-10120 mg/kg vs EAE+Vehicle) and this loss of myelin expression was strongly reverted by EHP-101 treatment. Moreover, EAE is associated with a loss in the expression of Olig2 in the Corpus callosum, a marker for oligodendrocyte differentiation, which was restored by EHP-101 treatment (FIG. 16G p<0.0001 EAE+Vehicle vs CFA; p=0.0008 EAE+EHP-101 20 mg/kg vs EAE+Vehicle). In addition. EHP-101 enhanced the expression of glutathione S-transferase pi (GSTpi), a cytosolic isoenzyme used as a marker for mature oligodendrocytes in the brain (FIG. 16H p=0.0222 EAE+EHP-101 20 mg vs EAE+Vehicle). These data are indicative of the potential of EHP-101 to prevent demyelination in an MS murine model.

EHP-101 Normalizes EAE Transcriptomic Signature at Spinal Cord

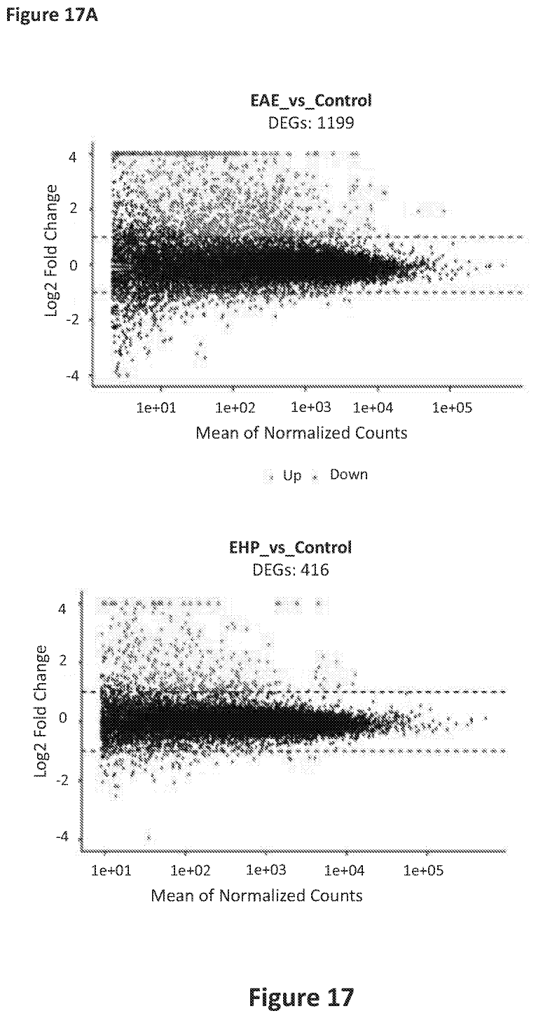

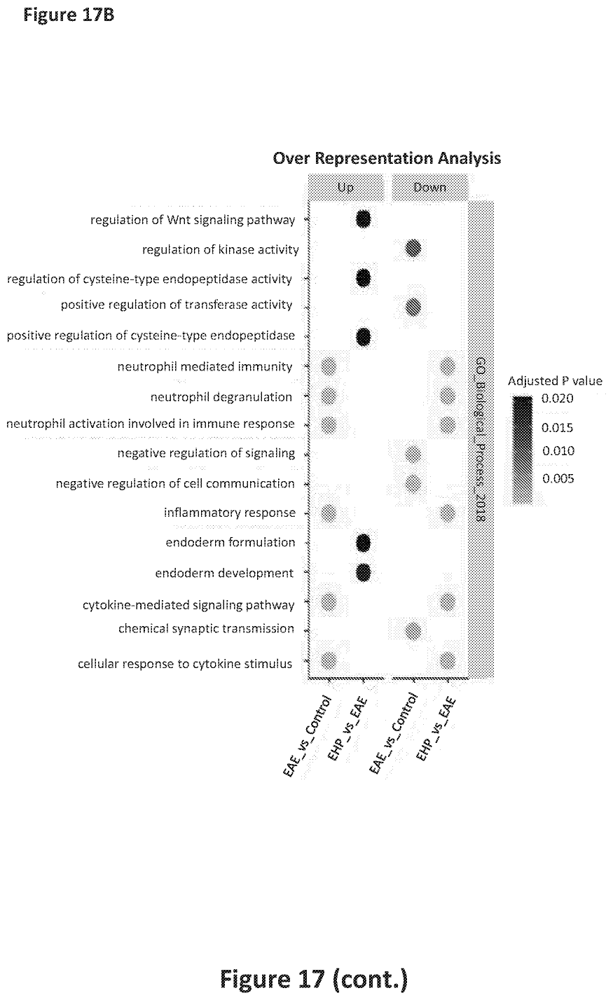

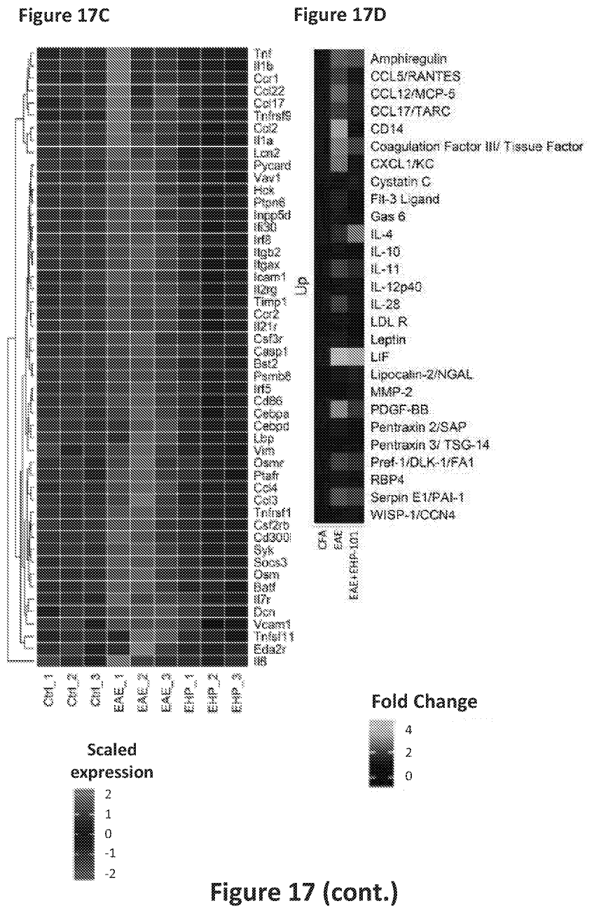

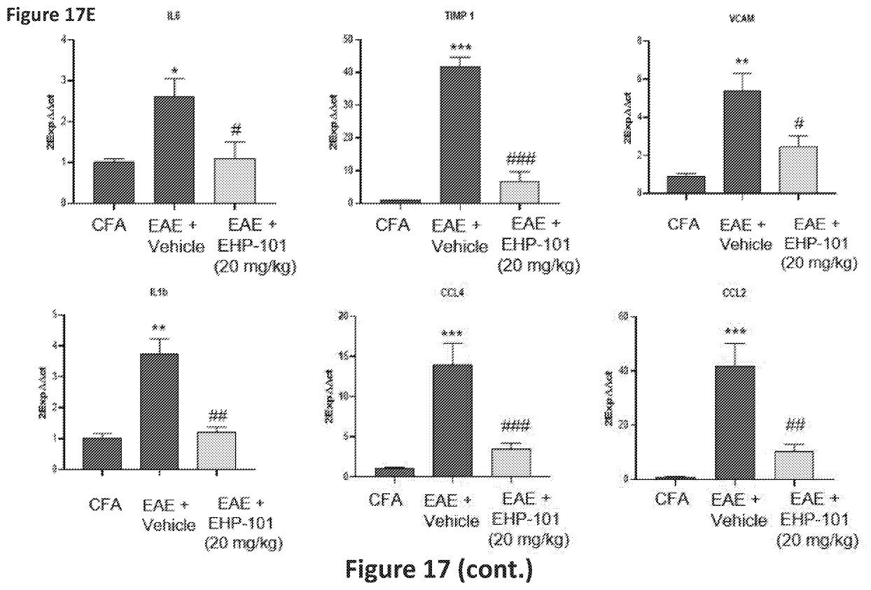

To evaluate the global expression changes produced by the EHP-101 treatment, an RNA-Seq analysis of the spinal cord from mice was performed in the following conditions: Control, EAE and EAE with EHP-101 treatment (20 mg/kg). Sequencing data for three biological replicates were obtained for each experimental group. Then, the transcriptomic profile was compared between the different conditions to get a first insight into the changes occurring at the model, with or without treatment. As expected, many changes were found, both in magnitude and significance in EAE mice compared to the group treated with EHP-101 (FIG. 17A). Then, to evaluate those changes at a biological level, an over-representation analysis was performed using genes that surpassed the cutoff of an adjusted P<0.05 and absolute fold change >2 in the EAE vs control and EAE+EHP-101 vs EAE comparisons. The more significant enrichments were found in the groups of upregulated genes by EAE and downregulated genes by the treatment. A complementary signature was observed between those two groups, where terms like “neutrophil mediated immunity”, “inflammatory response” or “cytokine-mediated signaling pathway” appeared, highlighting an anti-inflammatory effect of the EHP-101 treatment at the spinal cord (FIG. 17B). The heatmap in FIG. 17C represents genes from the “cytokine-mediated signaling pathway” that are induced by EAE and downregulated by EHP-101. Furthermore, to confirm this anti-inflammatory effect of EHP-101 in spinal cord, the gene expression by RT-PCR of several genes, such as I16, Timp1. Veam, I11b, Ccl4 and Ccl2, was determined. FIG. 17E shows that EHP-101 treatment downregulated the expression of these genes upregulated in EAE mice (I16: p=0.0360 EAE+Vehicle vs CFA; p=0.0451 EAE+EHP-101 20 mg/kg vs EAE+Vehicle; Timp1: p=0.0001 EAE+Vehicle vs CFA; p=0.0001 EAE+EHP-101 20 mg/kg vs EAE+Vehicle; VCAM: p=0.0058 EAE+Vehicle vs CFA, p=0.0381 EAE+EHP-101 20 mg/kg vs EAE+Vehicle; IL1b: p=0.018 EAE+Vehicle vs CFA; p=0.0027 EAE+EHP-101 20 mg/kg vs EAE+Vehicle; Ccl4: p=<0.0001 EAE+Vehicle vs CFA; p=<0.0001 EAE+EHP-101 20 mg/kg vs EAE+Vehicle: Ccl2: p=0.0003 EAE+Vehicle vs CFA; p=0.0054 EAE+EHP-101 vs EAE+Vehicle), thus validating the results found in the RNA-Seq analysis.

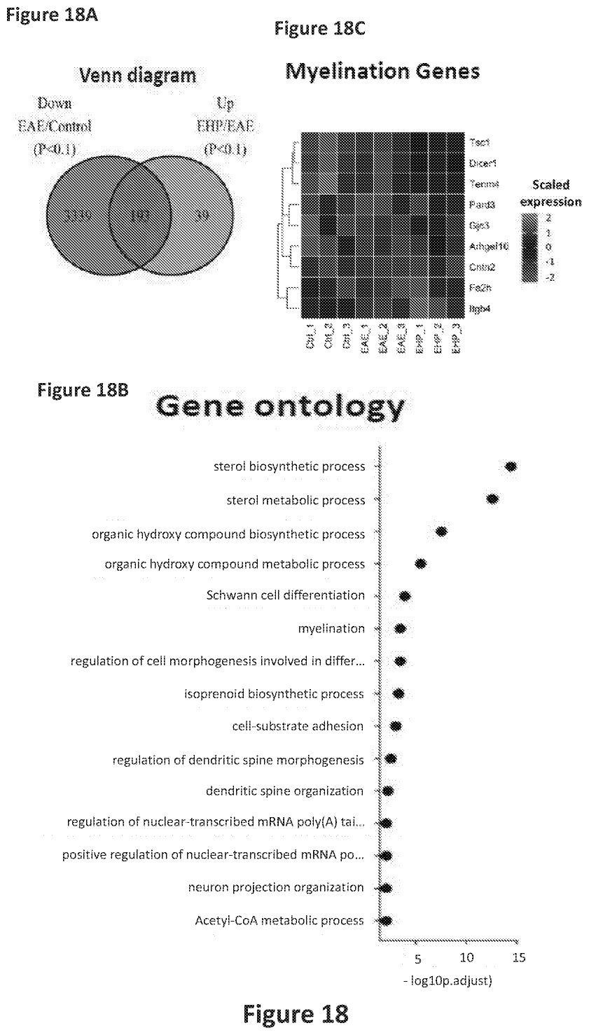

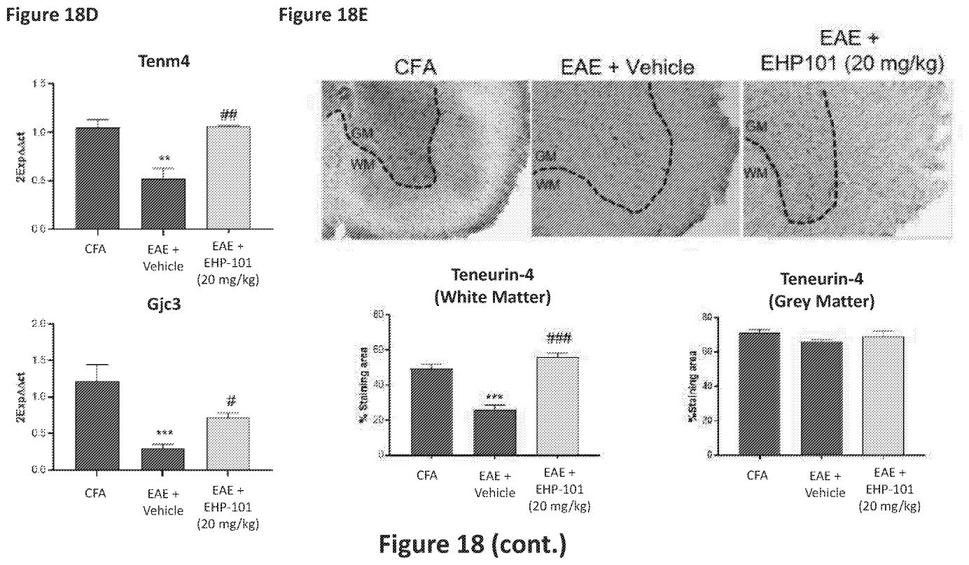

Next, a second analysis was performed to explore changes in the opposite direction to the pattern shown by the pro-inflammatory genes. Thus, down-regulated genes were selected at the EAE vs control comparison and up-regulated in EAE+EHP-101 vs EAE comparison. Both groups of genes were intersected to evaluate the overlap between them, resulting in a total of 193 genes downregulated in the untreated model that increased their expression in response to the treatment (FIG. 18A). Then a second functional analysis was performed, using the list of overlapping genes as input, to explore the most significantly enriched GO terms. As depicted in FIG. 18B, several terms related to the metabolic process of sterols and hydroxy compounds were found at the top of the list. However, given the background of the disease, focus was given to the “myelination” process. To explore the changes of features belonging to this annotation, the expression levels of genes that produced this result in the heatmap were depicted and are shown in FIG. 18C. This allowed us to identify several key genes of the myelination process that were restoring their levels with EHP-101 treatment. Interestingly, these results indicated that EHP-101 normalized the expression of several genes associated with oligodendrocyte function, such as Gap junction gamma-3 (Gjc3), also called Connexin 29, and Teneurin-4 (Tenm4) that were downregulated in EAE. These results are relevant since Tenm4 has been described as a critical regulator of oligodendrocyte differentiation and CMS myelination. To validate the transcriptomic analysis, the expression of Gjc4 and Tenm4 was studied by RT-PCR (FIG. 18D Tenm4; p=0.0020 EAE+Vehicle vs CFA; p=0.0032 EAE+EHP-101 20 mg kg vs EAE+Vehicle; Gjc3: p=0.0006 EAE+Vehicle vs CFA; p=0.0462 EAE+EHP-101 20 mg/kg vs EAE+Vehicle) and the protein levels by IHC. As depicted in FIG. 18E (p=<0.0001 EAE+Vehicle vs CFA; p=<0.0001 EAE+EHP-101 20 mg/kg vs EAE+Vehicle), a decrease of Tenm4 expression was observed in white matter of spinal cord compared to the CFA group which was prevented by EHP-101 treatment. Taken together, these results are indicative of the potential of EHP-101 to prevent demyelination in EAE model.

EHP-101 Accelerates Remyelination in Cuprizone-Challenged Mice

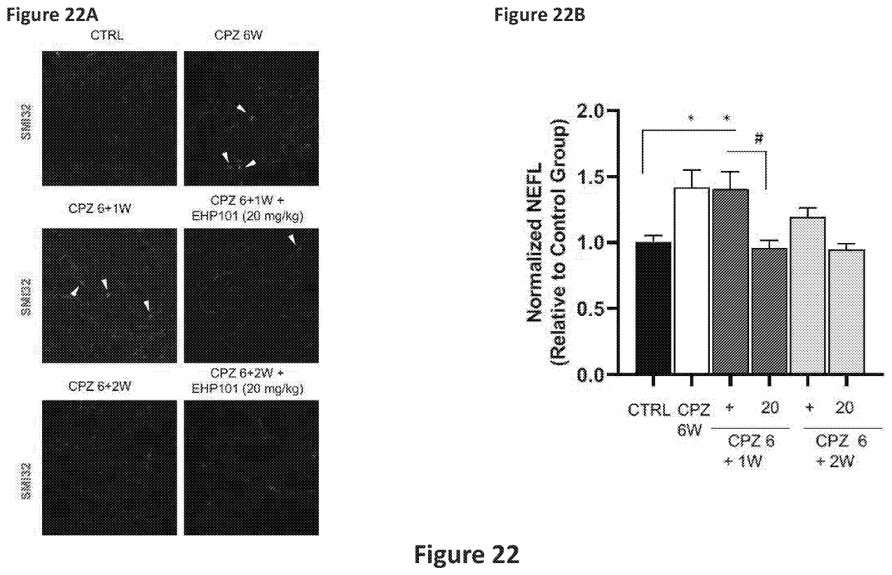

To evaluate the effect of EHP-101 on remyelination during the acute CPZ-induced demyelination protocol (FIG. 19A), brain coronal sections from animals after 6 weeks of CPZ 0.2% diet and 2 weeks of EHP-101 treatment were evaluated. In this model EHP-101 treatment started after removal of the CPZ diet to study the effect of EHP-101 on spontaneous remyelination. First, the evaluation of MBP was determined by CryoMyelin and IHC staining (FIG. 19R and FIG. 19C, respectively). Spontaneous recovery from demyelination was insignificant after 1 and 2 weeks in untreated mice but remyelination was significantly accelerated by EHP-101 treatment in both the Corpus callosum (FIG. 19D p=<0.0001 CPZ6W, CPZ6+1W, CPZ6+2W vs Control; p=<0.0001 CPZ6+1W EHP-101 20 mg kg vs CPZ6+1W; p=<0.0001 CPZ6+2W+EHP-101 20 mg/kg vs CPZ6+2W) and the cerebral cortex. (FIG. 19E p=<0.0001 CPZ6W, CPZ6+1W, CPZ6+2W vs Control; p=<0.0001 CPZ6+1W+EHP-101 20 mg/kg vs CP26+1W). Moreover, the effect of EHP-101 on neuroinflammation-associated glial activation was investigated by staining Iba-1 and GFAP+cells in the Corpus callosum. In control mice low level expression of Iba-1+ and GFAP+ cells was detected but mice exposed to CPZ showed microglial and astrocytic activation, which was attenuated by EHP-101 treatment (FIG. 20A and FIG. 20B). Quantitative assessment also showed a significant increase in the number of Iba1+ and GFAP+ cells in Corpus callosum upon CPZ intoxication. Microgliosis and astrocytic activation was ameliorated after 1 week of EHP-101 treatment (FIG. 20C p=<0.0001 GPZ6W, CPZ6+1W, CPZ6+2W vs Control; p=0.0017 CPZ6+1W+EHP-101 20 mg kg vs CPZ6+1W; FIG. 20D p=<0.0001 CPZ6W, CPZ6+1W vs Control; p=0.0017 CPZ6+2W vs Control). To examine the effects of EHP-101 on cuprizone-induced demyelination on axons in the Corpus callosum, the non-phosphorylated form of neurofilament proteins (SMI-32 staining) was investigated. Although SMI-32 immunoreactivity is normally seen in axons, its accumulation in axonal spheroids is a characteristic of axonal pathology. Increased SMI-32 labeling after 6 and 7 weeks of CPZ intoxication demonstrated that there was a significant effect on axons and this effect was ameliorated after 1 week of EHP-101 treatment (FIG. 22A). Moreover, plasma levels of Neurofilament Light Polypeptide (NEFL) were determined. As depicted in FIG. 2213, an increase of cuprizone-induced NEFL plasma levels was detected by ELISA studies after 6 and 7 weeks of CPZ exposure compared to control mice. It was also shown that one week of treatment with EHP-101 reduced the plasmatic levels of NEFL induced by cuprizone (FIG. 22B p=0.0113 CPZ 6W vs Control; p=0.0151 CPZ6+1W vs Control; p=0.0125 CPZ6+1W+EHP-101 20 mg/kg vs CPZ6+1W).

Natural products, including phytocannabinoids, have been successfully used for the development of synthetic and semisynthetic derivatives with improved bioactivities. The experiments described herein disclose the development of the compound VCE-004.8, a semi-synthetic derivative of cannabidiol, which is a dual agonist for PPARγ/CB2 that also inhibits the activity of HIF prolyl hydroxylases (PHDs). Therefore. VCE-004.8 is targeting several pathways that may have a positive effect in neuroinflammation and remyelination in EAE and Theiler's Murine Encephalomyelitis Virus-induced demyelinating disease. Herein described studies disclose the effect of EHP-101, an oral lipidic formulation of VCE-004.8, in the two most commonly used models of demyelination that are EAE and toxically induced demyelination via cuprizone.

EAE in C57B1/6 mice has generally been thought to predominantly target the spinal cord, leading to sensory and motor impairments. Nevertheless, it is also recognized that EAE involves other CNS structures including the cerebellum and the hippocampus. The data clearly indicate that EHP-101 is effective to alleviate neuroinflamination in the spinal cord, in the cerebral cortex and in the corpus callosum (CC). In the EAE model it was not possible to distinguish whether the effect of EHP-101 occurs at the peripheral immune system, at the CNS of both. It has been demonstrated that die brain blood barrier (BBB) is disrupted in EAE allowing the migration of autoimmune cells and molecules to the brain. However, it is likely that EHP-101 may exert anti-inflammatory effects by acting both at the peripheral immune system and at the CNS. For instance, EHP-101 showed anti-inflammatory activity in another autoimmune disease such as Systemic Sclerosis where the BBB is not affected and herein it was shown that EHP-101 also alleviates neuroinflammation in CPZ intoxicated mice. CPZ-induced demyelinating lesions are characterized by severe oligodendrocyte loss and demyelination with concomitant activation of microglia and astrocytes, but it does not induce BBB damage and lacks the characteristic T cell infiltration and consequently the peripheral autoimmune component of the disease.

The mechanism of action of EHP-101 in the remyclination process is still unknown but it can be probably related to the HIF pathway. Extensive experimental studies have revealed that activating HIF-1 by inhibiting the activation of PHDs can provide neuroprotection and perhaps remyelination mainly from the increased expression of HIF-1 target genes, which combat oxidative stress, improve blood oxygen and glucose supply, promote glucose metabolism, regulate iron homeostasis and block cell death signal pathways. Increasing HIF-1 activity may be an important potential strategy to prevent the onset or to ameliorate the pathogenesis of neurodegenerative diseases. Interestingly, the improvement of the myelination index was paralleled by enhancement of OPC proliferation, PDGFα-receptor expression, and precursor migration from the CC midline to the lateral parts followed by an induction of the expression of myelin protein. In addition, early astrogliosis in the demyelinated areas paralleled with a moderate stimulation of IGF-1 expression. IGF-1 synergizes with FGF-2 to stimulate oligodendrocyte progenitor entry into the cell cycle. This is of particular interest because IGF-1 induced HIF-1 activation that can be mimicked by VCE-004.8 in the brain, and PDGFα and FGF2 are also regulated by VCE-004.8-mediated activation of the HIF pathway.

Demyelination and partial axonal damage in MS lesions are closely associated with reactive activation of microglial cells which are seen in close contact with axons, that reveal acute axonal injury, such as the formation of axonal spheroids or a disturbance of fast axonal transport. Reactive microglia produce a large array of toxic and proinflammatory molecules, which triggers myelin destruction, oligodendrocyte deterioration, axon damage and even neuronal loss. Here it was found that oral EHP-101 also prevented microglia activation and demyelination in both spinal cord and brain suggesting that after oral absorption VCE-004.8 penetrates into the brain in EAE mice. Moreover, it was also found that EHP-101 preserves the axonal structure ameliorating the typical accumulation on spheroids of SMI-32 used as a marker of axonal damage in CPZ intoxicated mice. Again, this result suggests that VCE-004.8 can also cross the BBB that is not affected in the CPZ model.

Oligodendrocyte progenitor cells (OPCs) are produced from neuroepithelial stem cells and subsequently proliferate and migrate throughout the entire spinal cord. During differentiation, oligodendrocytes initiate expression of myelin proteins critical for the achievement of proper functioning of the CNS. Teneurin-4 (Tenm4) is a type II transmembrane protein that is highly expressed in the CNS and whose expression is induced in response to endoplasmic reticulum stress and has been suggested to be involved in bipolar disorder in humans. A mouse mutation, designated furue, which results in tremors and severe hypomyelmation of small-diameter axons, reduces oligodendrocyte differentiation especially in the spinal cord of the CNS, and it has been associated with the absence of Tenm4 expression. Thus, Tenm4 is a critical regulator of oligodendrocyte differentiation and CNS myelination. Herein it was shown for the first time that in EAE mice the expression of Tenm4 is downregulated in the spinal cord and the treatment with EHP-101 reverses this downregulation probably as the result of the anti-inflammatory activity of VCE-004.8.

In addition, oligodendrocytes are electrically and metabolically coupled through intercellular channels called gap junctions (GJs), composed of connexins Cx29, Cx32 and Cx47, with other oligodendrocytes as well as with astrocytes. This glial network of communication plays important roles in the homeostasis of brain function. Several studies have also provided the role of oligodendrocyte connexins in acquired demyelinating CNS disorders, in particular, MS and related experimental models. They also appear to have a regulatory role in neuroinflammation as their absence further aggravates inflammatory demyelination. Again, the results showed that EHP-101 prevented the downregulation of Gjc3 (connexin 29) expression in EAE mice vs control mice. In the light of the relevance of Tenm4 and Gjc3 for oligodendrocyte function and myelin preservation, the results further support the potentiality of EHP-101 to be developed as a novel treatment of MS.

In conclusion, the disclosed studies provide the protective effect of EHP-101 against demyelination and its capability to enhance remyelination. These results open new strategies for the treatment of multiple sclerosis, since novel therapies aimed to axonal remyelination are urgently needed.

In summary, MS is characterized by a combination of inflammatory and neurodegenerative processes in the spinal cord and the brain. Naruxal and synthetic cannabinoids such as VCE-004.8 have been studied in preclinical models of MS and, therefore, represent promising candidates for drug development. VCE-004-8 is a multi target synthetic cannabidiol derivative acting as a dual PPARγ/CB2 ligand agonist that also activates the HIF pathway. EHP-101 is an oral lipidic formulation of VCE-004.8 that showed efficacy in other preclinical models of autoimmune diseases.

The efficacy of EHP-101 in vivo was evaluated in two murine models of MS such as experimental autoimmune encephalomyelitis (EAE) and cuprizone-induced demyelination. In EAE the transcriptomic analysis was performed by RNA-Seq and qPCR, and inflammatory and myelination markers were detected by immunohistochemistry (IHC) and confocal microscopy in both models of MS.

EHP-101 alleviates clinical symptomatology in EAE and transcriptomic analysis demonstrated that EHP-101 prevented the expression of many inflammatory genes closely associated with MS pathophysiology in the spinal cord. EHP-101 normalized the expression of several genes associated with oligodendrocyte function such as Teneurin 4 (Tenm4) and Gap junction gamma-3 (Gjc3) that were down regulated in EAE. EHP-101 treatment prevented microglia activation and demyelination in both the spinal cord and the brain. Moreover, EAE was associated with a loss in the expression of Olig2 in the Corpus callosum, a marker for oligodendrocyte differentiation, which was restored by EHP-101 treatment. In addition, EHP-101 enhanced the expression of glutathione S-transferase pi (GSTpi), a marker for mature oligodendrocytes in the brain. It was also found that a diet containing 0.2% of cuprizone for six weeks induced a clear loss of myelin in the brain measured by Cryomyelin staining and MPB expression. Moreover, EHP-101 also prevented cuprizone-induced microglial activation and astrogliosis, reduced axonal damage and decreased plasma levels of Neurofilament Light Polypeptide (NEFL).

The results disclosed herein provide evidence that EHP-101 showed potent anti-inflammatory activity, prevented demyclination and enhanced remyclination. Therefore. EHP-101 represents a promising drug candidate for the potential treatment of different forms of MS.

### Example 21

### Myelin Assessment in Grey and White Matter

Myelin assessment in grey and white matter was evaluated via: (1) PLP staining and density in the hippocampus and cortex; and (2) PPD staining and manual counts in the corpus callosum. The myelin assessment in grey and white matter model summary is demonstrated in Table 19. VCB-004.8 was formulated into EHP-101 and daily PO administration of EHP-101 was constructed.

TABLE 19
Myelin Assessment in Grey and White Matter Model Summary

| | | Demyelination | Remyelination | |
|---|---|---|---|---|
| Group | Mice N = | Paradigm | Paradigm | Harvest |
| 1 | 5 | 12 + 6 weeks | Age Match (No Dose) | 18 weeks |
| | | Age Match | | |
| 2 | 15 | 12 + 0 C/R | N/A | 12 weeks |
| 3 | 15 | 12 + 6 C/R | Vehicle Control (PO) | 18 weeks |
| 4 | 15 | 12 + 6 C/R | Test Compound | 18 weeks |
| | | | Concentration A (PO) | |
| 5 | 15 | 12 + 6 C/R | Test Compound | 18 weeks |
| | | | Concentration B (PO) | |
| 6 | 15 | 12 + 6 C/R | Test Compound | 18 weeks |
| | | | Concentration C (PO) | |

### Grey Matter Remyelination

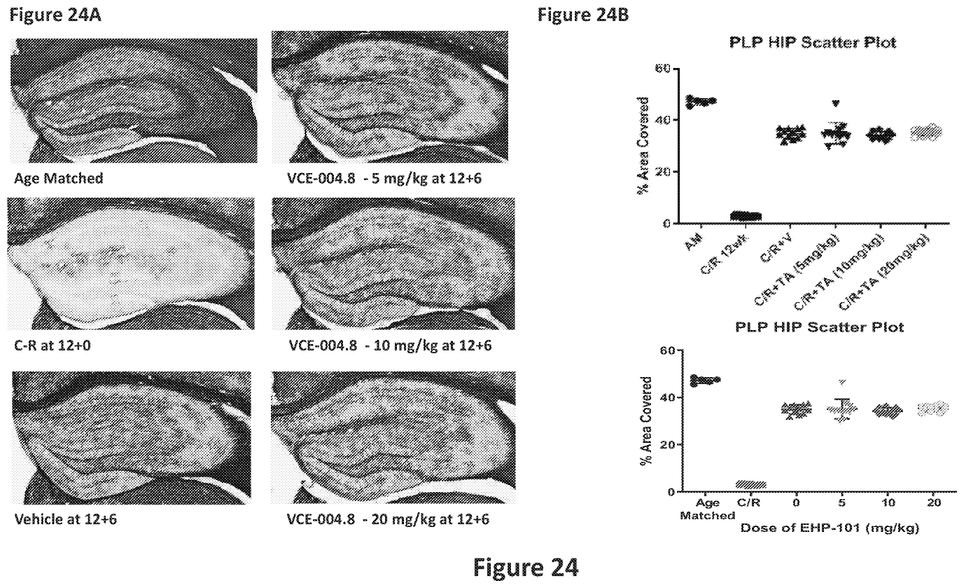

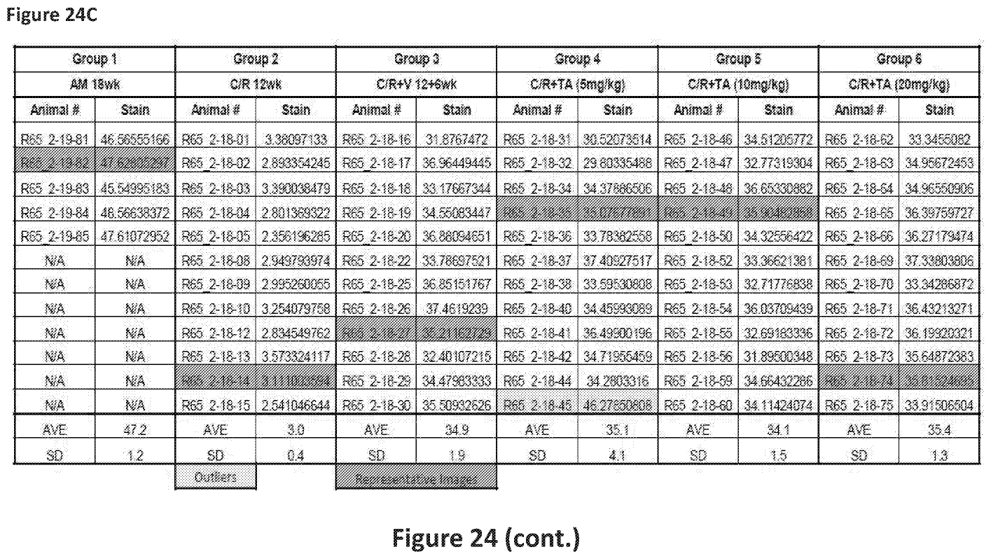

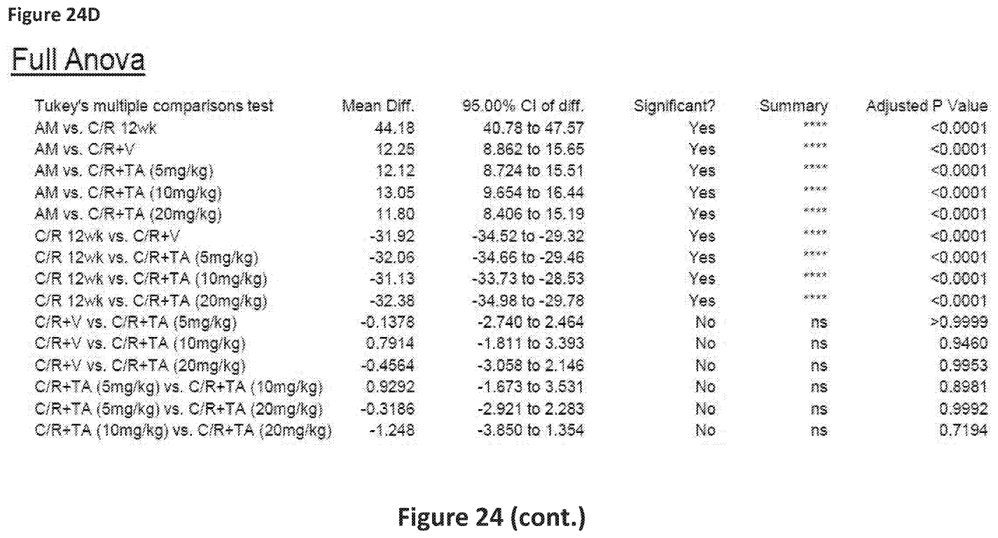

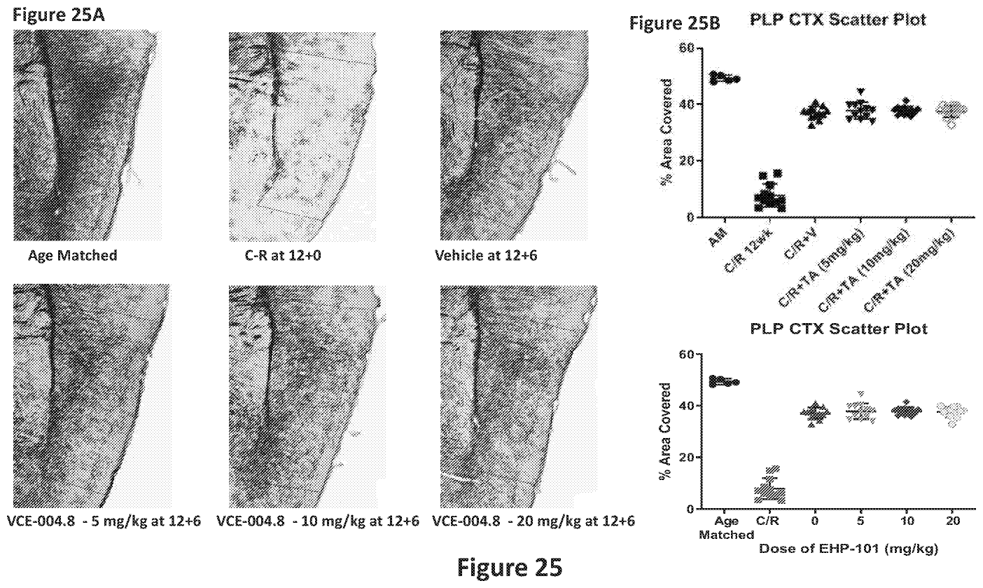

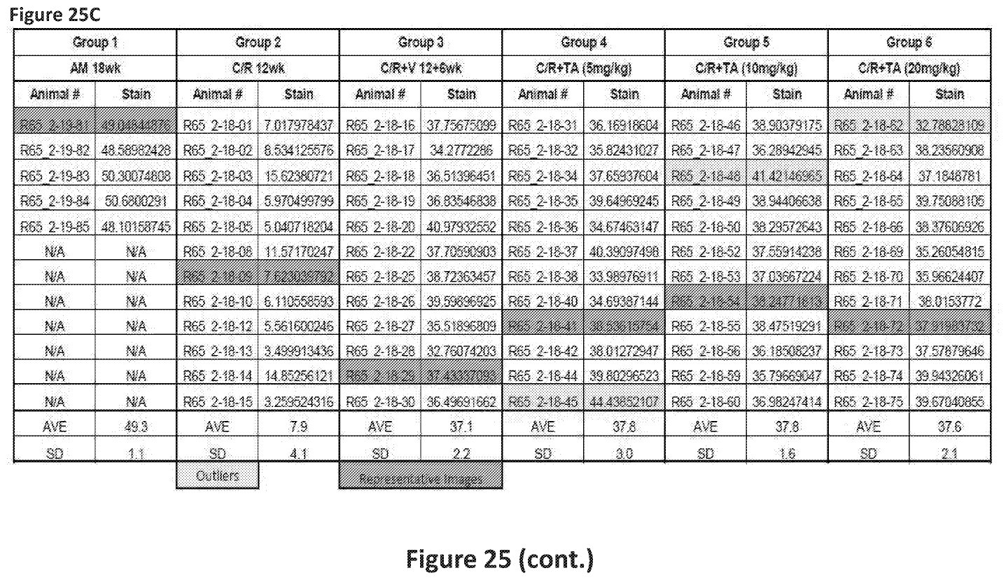

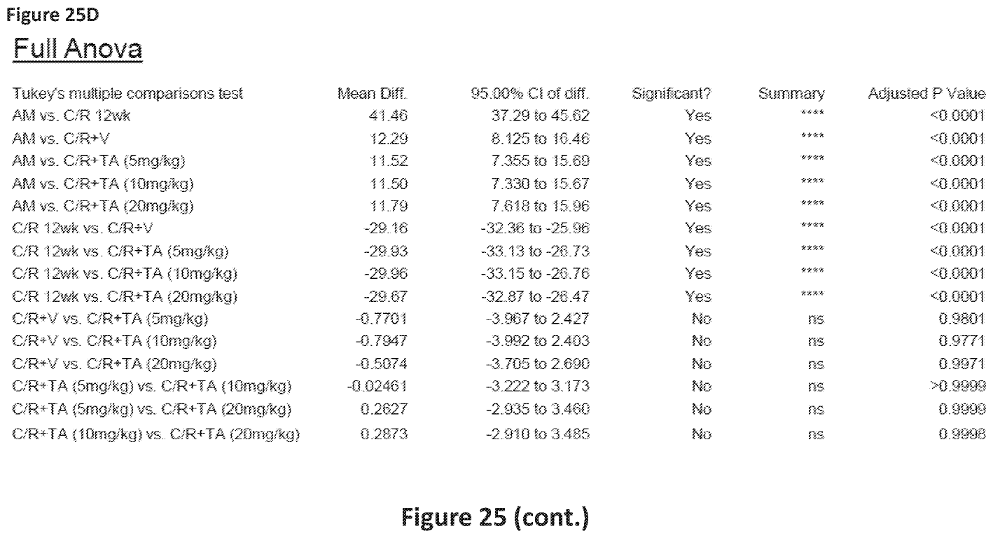

As shown in FIG. 24, PLP staining in the hippocampus and quantification of PLP in the hippocampus demonstrated that EHP-101 treated animals showed no change in the area of PLP staining in the hippocampus compared to vehicle control. Furthermore, PLP staining in the cortex and quantification of PLP in the cortex demonstrated that EHP-101 treated animals at all dose strengths showed no change in the area of PLP staining in the cortical region compared to vehicle control (FIG. 25).

### White Matter Remyelination

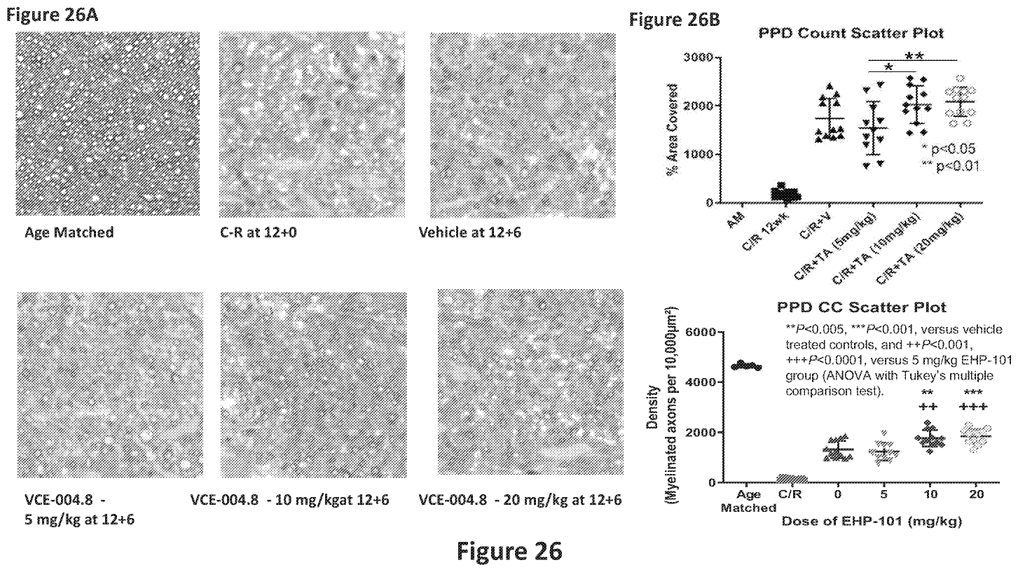

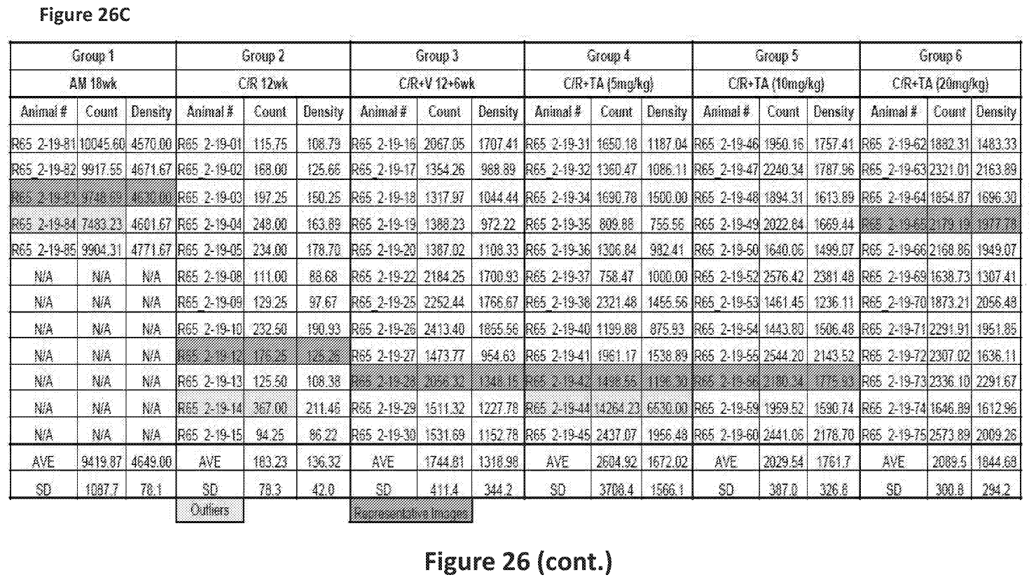

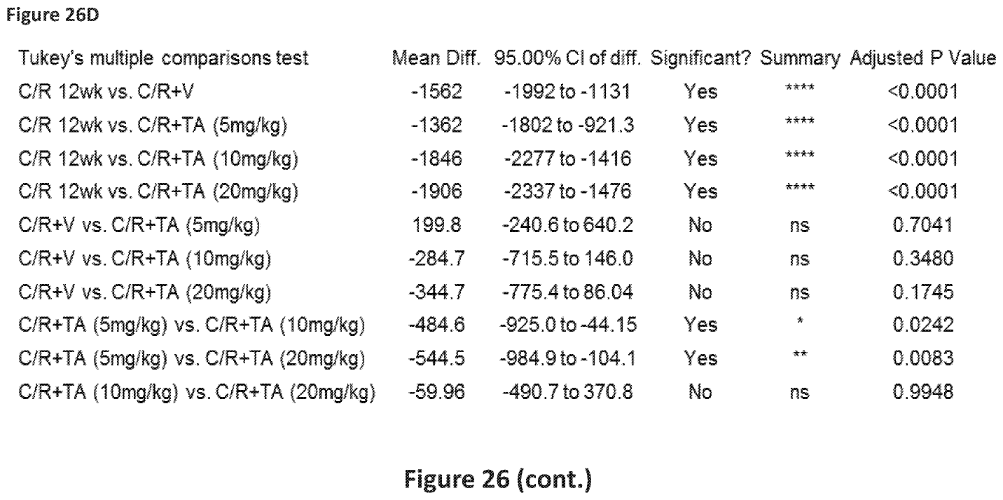

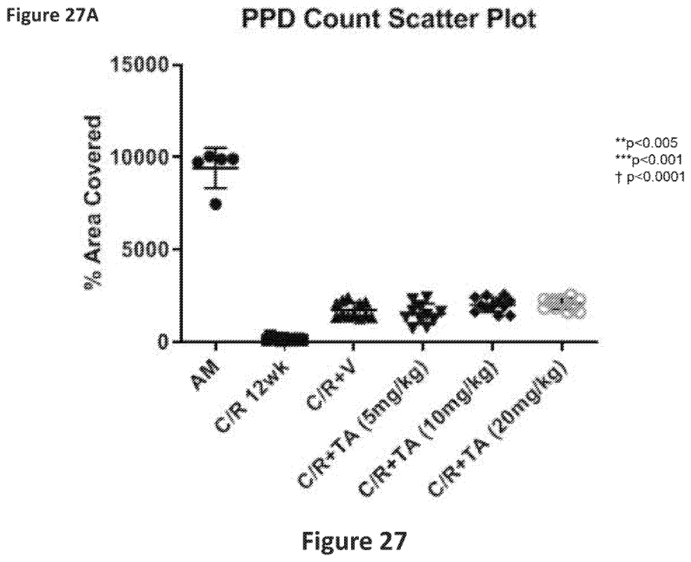

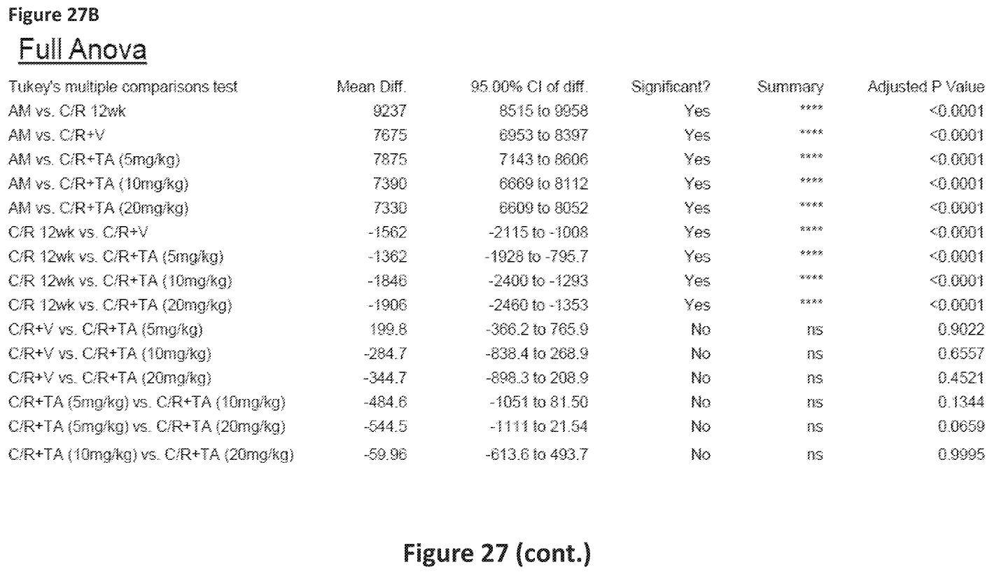

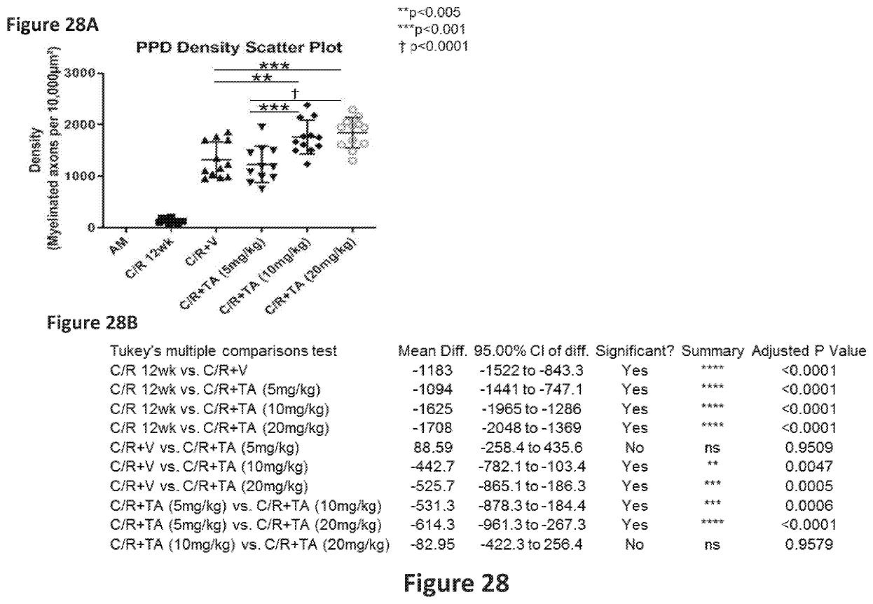

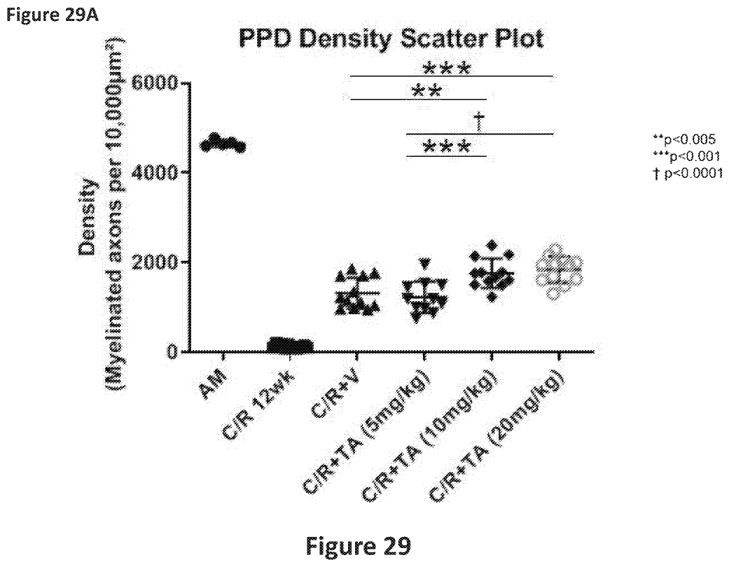

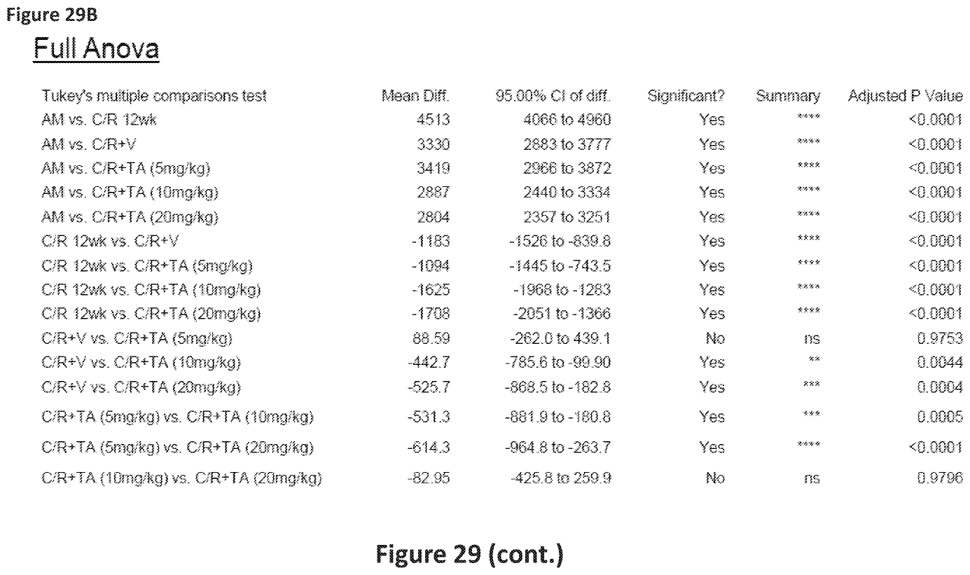

As shown in FIG. 26 through FIG. 29, PPD staining in the corpus callosum and the myelinated axons in the corpus callosum demonstrated that although EHP-101 treatments did not show a significant increase in myelinated axons compared to control, there was a significant difference between the two higher groups when compared to the lowest tested group of the test article. Moreover, FIG. 26A, FIG. 28A, and FIG. 28B demonstrated that the higher doses tested of EHP-101 treatments showed a significant increase in the density of myelinated axons compared to control. There was also a significant difference between the two higher groups when compared to the lowest tested group of the test article.

In summary, animals demyelinated very well, and as expected, as demonstrated by the lack of myelin at the 12+0 time point in the Cup-Rap treatment paradigm. There was no significant increase in myelination in the hippocampal area at any dose of VCE-004.8. There appeared to be no significant increase in cortical myelination with any dose of VCE-004.8. VCE-004.8 appeared to have a dose related effect on myelination in white matter in the observed region of the corpus callosum. Increased levels of myelinated axons were observed at higher doses.

### Example 22

### Oral Administration of EHP-101 Promotes Remyelination in White Matter in the Cuprizone/Rapamycin Mouse Model of Multiple Sclerosis

As stated above, EMP-101 is an oral lipidic formulation of VCE-004.8, a novel non-psychotropic aminoquinone derivative of synthetic cannabidiol that recently completed a Phase I clinical study. VCE-004.8 is a dual agonist of the PPARγ and CB₂ receptors with potent anti-inflammatory activity. VCE-004.8 has also demonstrated activation of the HIF pathway in human microvascular endothelial cells, oligodendrocytes, and microglia. In vivo, EHP-101 has been shown to prevent demyelination in different murine models of MS and was also shown to induce remyelination in brain in a mouse cuprizone model with less complete demyelination, faster remyelination, and only a 2-week treatment window.

As such, the present example focuses on the evaluation of the potential of oral administration of EHP-101 to promote remyelination in gray and white matter in the cuprizone/rapamycin (C/R) mouse model of extensive demyelination with slower spontaneous remyelination and a 6-week treatment window.

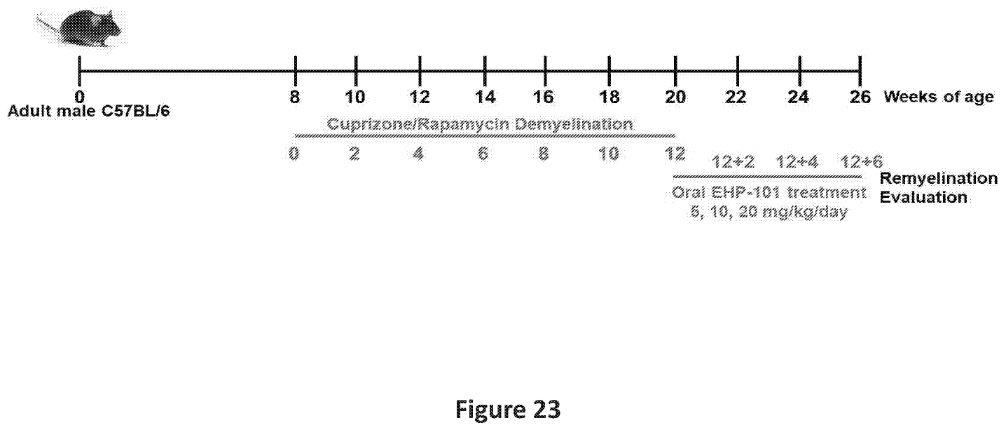

Male C57BL/6J (n=5 or 12/group) were treated for 12 weeks with C/R to cause demyelination of white and gray matter regions of the brain. The mice were then orally administered EHP-101 at 0, 5, 10, and 20 mg/kg/day for 6 weeks (FIG. 23). Thereafter, the brains were harvested and processed for immunohistochemical staining and quantification of myelinated axons in gray matter (hippocampus (HIP), cerebral cortex (CTX)) by proteolipid protein (PLP) staining and white matter (corpus callosum CC)) by paraphenylenediamine (PPD) staining.

After 12 weeks of C/R administration, there was a near complete axonal tissue demyelination in the cortex and hippocampus as quantified by a decrease in myelin proteolipid protein staining and in the corpus callosum as quantified by paraphenylenediamine staining when compared to age-matched controls. There was no significant change in the area of PLP staining in the hippocampus and cerebral cortex after EHP-101 treatment. There was no significant increase in gray matter myelination when compared to vehicle control following oral administration of EHP-101 at ≥5 mg/kg/day. In white matter, there was a dose-dependent increase in the levels of myelinated axons in the corpus callosum. Statistically significant, increases in the density of myelinated axons were observed after administration of EHP-101 at 10 (p<0.005) and 20 mg/kg/day (p<0.001) relative to controls.

In summary, in the augmented cuprizone model of demyelination, oral administration of EHP-101 induced significant remyelination of demyelinated axons in white matter but not gray matter. EHP-101 induced a significant, dose-related increase in the density of PPD staining in the corpus callosum. These data support the advancement of EMP-101 into Phase 2 clinical studies as a therapy for treating MS patients.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims ate intended to be construed to include all such embodiments and equivalent variations.

1. A composition comprising at least one compound of Formula (I), or a derivative thereof. 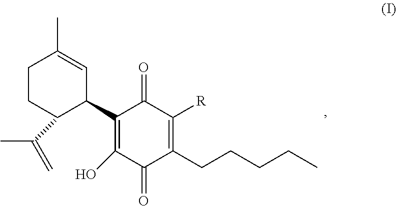
wherein R is the nitrogen atom of a group independently selected from a linear or branched alkylamine, an aryl amine, an arylalkylamine, a heteroarylamine, a heteroarylalkylamine, a linear or branched alkenylamine, a linear or branched alkynylamine, or NH₂,
in a pharmaceutical vehicle,
wherein the pharmaceutical vehicle is selected from the group consisting of aqueous buffers, solvents, co-solvents, cyclodextrin complexes, lipid vehicles, and any combination thereof.

2. The composition of claim 1, wherein the composition is selected from a liquid formulation, a suspension formulation, a nanosuspension formulation, an emulsion formulation and a dry powder formulation.

3. (canceled)

4. (canceled)

5. (canceled)

6. (canceled)

7. The composition of claim 2, wherein the composition is a dry powder formulation that is compressed into a tablet.

8. The composition of claim 1, wherein the composition is a solution, a gel, a lotion, a paste, an ointment, an emollient, a liposome, a nanosphere, a skin tonic, a mouth wash, an oral rinse, a mousse, a spray, a pack, a capsule, a granule, a patch, an occlusive skin agent, or any combination thereof.

9. The composition of claim 1, wherein said compound of Formula (I) is selected from the group consisting of:

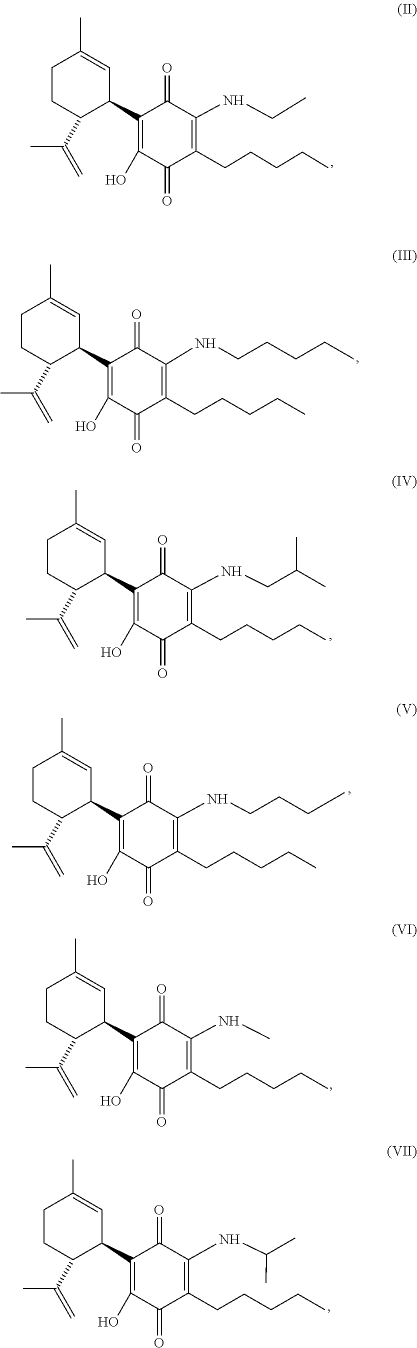

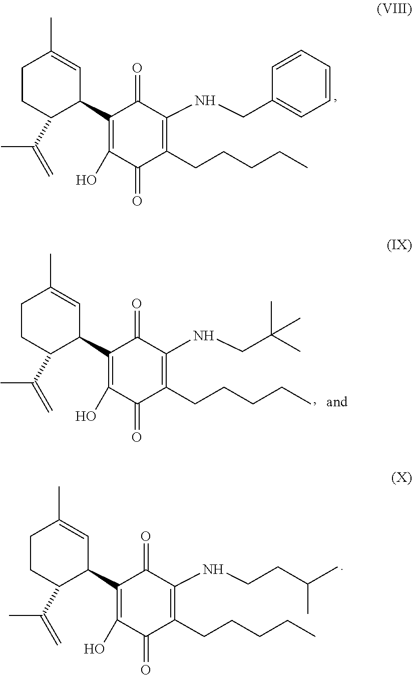

10. The composition of claim 1, wherein the pharmaceutical vehicle is selected from the group consisting of aqueous buffers, solvents, co-solvents, cyclodextrin complexes, lipid vehicles, and any combination thereof, and further comprising at least one stabilizer, emulsifier, polymer, and any combination thereof.

11. (canceled)

12. The composition of claim 10, wherein the solvent is selected from the group consisting of acetone, ethyl acetate, acetonitrile, pentane, hexane, heptane, methanol, ethanol, isopropyl alcohol, dimethyl sulfoxide (DMSO), water, chloroform, dichloromethane, diethyl ether, PEG400, Transcutol (diethylene glycomonoethyl ether), MCT 70, Labrasol (PEG-8 caprylic/capric glycerides), Labrafil M1944CS (PEG 5 Oleate), propylene glycol, Transcutol P, PEG400, propylene glycol, glycerol, Captex 300, Tween 85, Cremophor EL, Maisine 35-1, Maisine CC, Capmul MCM, maize oil, and any combination thereof.

13. The composition of claim 10, wherein the co-solvent is selected from the group consisting of acetone, ethyl acetate, acetonitrile, pentane, hexane, heptane, methanol, ethanol, isopropyl alcohol, dimethyl sulfoxide (DMSO), water, chloroform, dichloromethane, diethyl ether, PEG400, Transcutol (diethylene glycomonoethyl ether), MCT 70, Labrasol (PEG-8 caprylic/capric glycerides), Labrafil M1944CS (PEG 5 Oleate), propylene glycol, Transcutol P, PEG400, propylene glycol, glycerol, Captex 300, Tween 85, Cremophor EL, Maisine 35-1, Maisine CC, Capmul MCM, maize oil, and any combination thereof.

14. The composition of claim 10, wherein the cyclodextrin complexes is selected from the group consisting of methyl-β-cyclodextrin, methyl-y-cyclodextrin, HP-β-cyclodextrin, HP-γ-cyclodextrin, SBE-β-cyclodextrin, α-cyclodextrin, γ-cyclodextrin,6-O-glucosyl-β-cyclodextrin, and any combination thereof.

15. The composition of claim 10, wherein the stabilizer is selected from the group consisting of Pharmacoat 603, SLS, Nisso HPC-SSL, Kolliphor, PVP K30, PVP VA 64, and any combination thereof.

16. The composition of claim 10, wherein the polymer is selected from the group consisting of HPMC-AS-MG, HPMC-AS-LG, HPMC-AS-HG, HPMC, HPMC-P-55S, HPMC-P-50, methyl cellulose, HEC, HPC, Eudragit L100, Eudragit E100, PEO 100K, PEG 6000, PVP VA64, PVP K30, TPGS, Kollicoat IR, Carbopol 980NF, Povocoat MP, Soluplus, Sureteric, Pluronic F-68, and any combination thereof.

17. The composition of claim 10, wherein the antioxidant is selected from the group consisting of Vitamin A, Vitamin C, Vitamin E, Coenzyme Q10, manganese, iodide, melatonin, alpha-carotene, astaxanthin, beta-carotene, canthaxanthin, cryptoxanthin, lutein, lycopene, zeaxanthin, polyphenol antioxidant, flavonoid, flavones, apigenin, luteolin, tangeritin, flavonol, isorhammetin, kaempferol, myricetin, proanthocyanidin, quercetin, flavanone, eriodictyol, hesperetin, naringenin, flavanol, catechin, gallocatechin, gallate esters, epicatechin, epigallocatechin, theaflavin, thearubigin, isoflavone phytoestrogen, daidzein, genistein, glycitein, stilbenoid, resveratrol, pterostilbene, anthocyanin, cyanidin, delphinidin, malvidin, pelargonidin, peonidin, petunidin, chicoric acid, caffeic acid, chlorogenic acid, ferulic acid, cinnamic acid, ellagic acid, ellagitannin, gallic acid, gallotannin, rosmarinic acid, salicylic acid, curcumin, flavonolignan, silymarin, xanthone, eugenol, capsaicin, bilirubin, citric acid, oxalic acid, phytic acid, n-acetylcysteine, R-alpha-lipoic acid, and any combination thereof.

18. The composition of claim 10, wherein the lipid vehicle is selected from the group consisting of Captex 300, Tween 85, Cremophor EL, Maisine 35-1, Maisine CC, Capmul MCM, corn oil, and any combination thereof.

19. The composition of claim 10, wherein the lipid vehicle is an oil.

20. The composition of claim 19, wherein the lipid vehicle is an oil mixture comprising at least two oils.

21. The composition of claim 20, wherein the oil mixture is a mixture of Maisine CC and maize oil.

22. The composition of claim 21, wherein the mixture of Maisine CC and maize oil comprises 50 Maisine CC:50 maize oil v/v.

23. The formulation of claim 2, wherein the pharmaceutical vehicle is an oil.

24. The formulation of claim 2, wherein the pharmaceutical vehicle is an oil mixture.

25. The formulation of claim 24, wherein the oil mixture is a mixture of Maisine CC and maize oil.

26. The formulation of claim 25, wherein the mixture of Maisine CC and maize oil comprises 50 Maisine CC:50 maize oil v/v.

27. A method of treating a condition or disease in a subject in need thereof, wherein the method comprises administering to the subject a therapeutically effective amount of at least one compound of Formula (I), or a derivative thereof from the composition of claim 1.

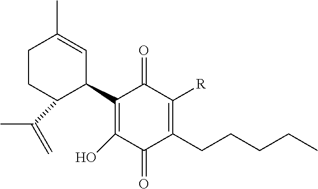

28. The method of claim 27, wherein said compound of Formula (I) is independently selected from the group consisting of:

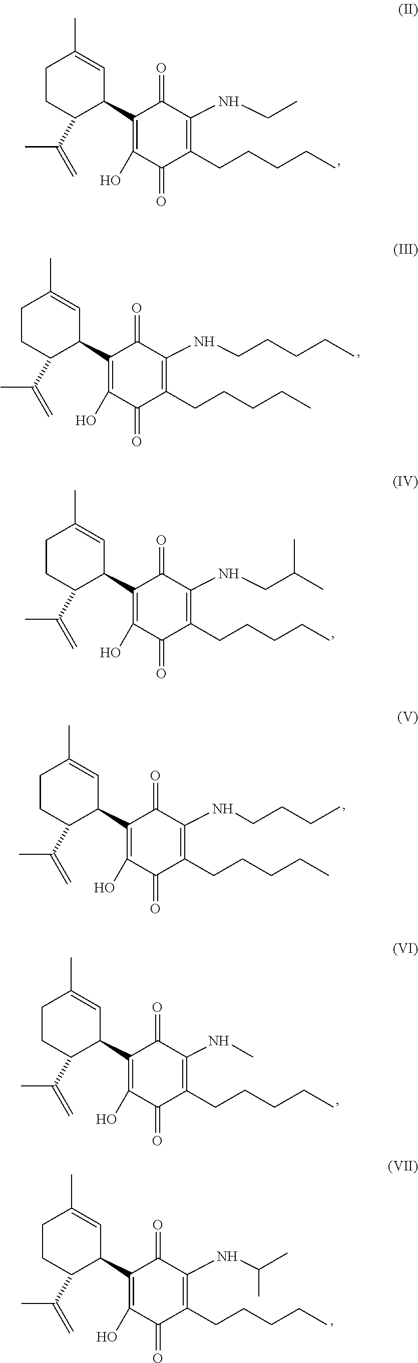

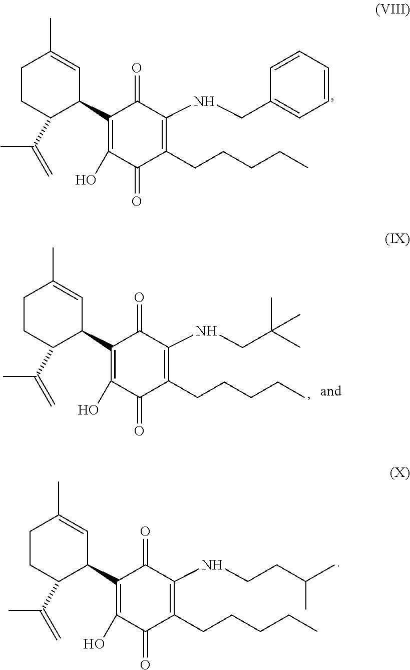

29. (canceled)

30. (canceled)

31. The method of claim 27, wherein the condition or disease is selected from the group consisting of autoimmune disease, demyelinating disease, fibrosis, inflammatory-related disorder, neurological disorder and any combination thereof.

32. The method of claim 27, wherein the condition or disease is selected from the group consisting of systemic sclerosis, myelinoclastic disorder, multiple sclerosis, neuromyelitis optica, central nervous system neuropathy, central pontine myelinolysis, myelopathy, leukoencephalopathy, leukodystrophy, peripheral neuropathy, Guillain-Barre syndrome, anti-MAG peripheral neuropathy, Charcot-Marie-Tooth disease, progressive inflammatory neuropathy, and any combination thereof.

33. The method of claim 27, wherein the condition or disease is multiple sclerosis or systemic sclerosis.

34. The method of claim 27, wherein said compound of Formula (I) is administered using oral, topical, sublingual, intramuscular, transmucosal, buccal, subcutaneous, rectal, intravenous, intramedullary, intrathecal, intraventricular, intraperitoneal, intranasal, or intraocular administration.

35. (canceled)

36. (canceled)

37. (canceled)

38. (canceled)

39. The method of claim 27, wherein said compound of Formula (I) is administered in combination with another therapeutic agent.

40. A liquid formulation, comprising compound of Formula (VIII), or a derivative thereof, in a pharmaceutical vehicle, wherein the pharmaceutical vehicle is 50:50 v/v Maisine CC:maize oil mixture.

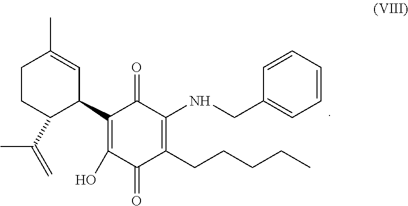

41. A method of treating a multiple sclerosis or systemic sclerosis in a subject in need thereof, wherein the method comprises administering to the subject a therapeutically effective amount of the compound of Formula (VIII) or a formulation thereof, or a derivative thereof,

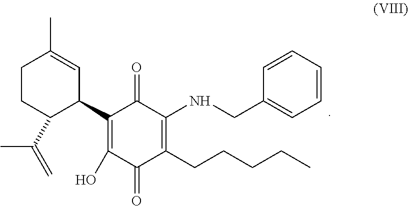

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)
